(19) United States
(12) Patent Application Publication
(10) Pub. No.: US 2005/0288307 A1
(43) Pub. Date: Dec. 29, 2005

(54) Anti-viral compounds

(76) Inventors: Shudong Wang, Forfar (GB); Christopher Meades, Dundee (GB); Gavin Wood, Cupar (GB); David Blake, Monifieth (GB); Peter Martin Fischer, Arbroath (GB)

Correspondence Address: LAHIVE & COCKFIELD, LLP., 28 STATE STREET, BOSTON, MA, 02109, US

(21) Appl. No.: 11/129,198

(22) Filed: May 13, 2005

Related U.S. Application Data
(63) Continuation of application No. PCT/GB03/04977, filed on Nov. 14, 2003.

(30) Foreign Application Priority Data
Nov. 14, 2002 (GB) 0226582.5

(51) Int. Cl. A61K 31/506

(52) U.S. Cl. 514/256; 514/275

(57) ABSTRACT

The present invention relates to the use of 2-substituted 4-heteroaryl-pyrimidines and related compounds in the treatment of viral disorders.

### RELATED APPLICATIONS

This application is a continuation of PCT/GB2003/004977, filed on Nov. 14, 2003, which claims priority to GB 0226582.5, filed on Nov. 14, 2002. The entire contents of each of these applications are hereby incorporated herein by reference.

### BACKGROUND

Certain 4,5,6-substituted-N-(substituted-phenyl)-2-pyrimidineamines having anti-asthmatic properties are disclosed in EP-A-233,461. Certain 4-heteroaryl-N-(3-substituted-phenyl)-2-pyridineamines possessing anti-proliferative properties and inhibiting protein kinases C, epidermal growth factor receptor-associated tyrosine protein kinase (EGF-R-TPK), as well as CDK1/cyclin B have been disclosed in WO95/09847 wherein the exemplified heteroaryl groups are pyridyl and indolyl. J. Med. Chem. (1993) Vol. 36, pages 2716-2725, Paul, R. et al, discloses a further class of phenyl amino-pyrimidines possessing anti-inflammatory activity. These compounds include mono-substituted 2-thienyl groups and dimethyl-3-furyl groups at the 4-position of the pyrimidine ring.

Further 2-substituted 4-heteroaryl-pyrimidines having antiproliferative activity are disclosed in WO01/72745 and International Patent Application No. PCT/GB2002/004383, both in the name of Cyclacel Limited. To date, however, there has been no teaching or suggestion that any of the above-disclosed 2-substituted 4-heteroaryl-pyrimidines have therapeutic applications in the treatment of viral disorders.

### STATEMENT OF INVENTION

The present invention relates to the use of 2-substituted 4-heteroaryl-pyrimidines in the treatment of antiviral disorders.

The present invention relates to the use of one or more compounds of formula I 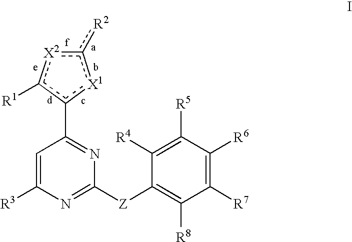
wherein:
(A) one of X¹ and X² is S, and the other of X¹ and X² is N;
   “a” is a single bond; and
   “b”, “c”, “d”, “e” and “f” are single or double bonds so as to form a thiazolyl ring;
   R² is independently as defined below for R¹ and R³; or
(B) one of X¹ and X² is S, and the other of X¹ and X² is NR⁹;
   “a” and “d” are each double bonds; and
   “b”, “C”, “e” and “f” are each single bonds;
   R² is oxo;
   R⁹ is H or alkyl;
      where:
      Z is NH, NHCO, NHSO₂, NHCH₂, CH₂, CH₂CH₂, or CH═CH;

R¹ and R³ are independently H, alkyl, aryl, aralkyl, heterocycle, halogeno, NO₂, CN, OH, alkoxy, aryloxy, NH₂, NH-alkyl, N—(R′)(R″), NH-aryl, N-(aryl)₂, NHCOR′, COOH, COO-alkyl, COO-aryl, CONH₂, CONH—R′, CON—(R′)(R″), CONH-aryl, CON-(aryl)₂, SO₃H, SO₂NH₂, CF₃, CO—R′, or CO-aryl, wherein said alkyl, NH-aryl, COO-alkyl, NH-alkyl, aryl, aralkyl and heterocycle groups may be further substituted with one or more groups selected from halogeno, NO₂, CN, OH, O-methyl, NH₂, COOH, N—(R′)(R″), CONH₂ and CF₃;

R⁴, R⁵, R⁶, R⁷, and R⁸ are independently from each other H, substituted or unsubstituted lower alkyl, halogeno, NO₂, CN, OH, substituted or unsubstituted alkoxy, NH₂, NH—R′, alkyl-aryl, alkyl-heteroaryl, NH(C═NH)NH₂, N(R′)₃⁺, N—(R′)(R″), COOH, COO—R′, CONH₂, CONH—R′, CON—(R′)(R″), SO₃H, SO₂NH₂, CF₃ or (CH₂)ₙO(CH₂)ₘNR′R″, (CH₂)ₙCO₂(CH₂)ₘOR′″ wherein n is 0, 1, 2 or 3 and m is 1, 2 or 3;

wherein R′ and R″ are each independently substituted or unsubstituted alkyl or alkenyl groups that may be the same or different; and pharmaceutically acceptable salts thereof; in the preparation of a medicament for use in the treatment of a viral disorder.

Preferred Embodiments

As used herein the term “alkyl” includes both straight chain and branched alkyl groups having from 1 to 8 carbon atoms, e.g. methyl, ethyl propyl, isopropyl, butyl, isobutyl, tert-butyl, pentyl, hexyl etc. and the term “lower alkyl” is similarly used for groups having from 1 to 4 carbon atoms.

As used herein, the term “aryl” refers to a monoaromatic or polyaromatic system, wherein said polyaromatic system may be fused or unfused. Preferably, the term “aryl” includes groups having from 6 to 10 carbon atoms, e.g. phenyl, naphthyl etc. The term “aryl” is synonymous with the term “aromatic”.

The term “aralkyl” is used as a conjunction of the terms alkyl and aryl as given above.

The term “heterocycle” refers to a saturated or unsaturated cyclic group containing one or more heteroatoms in the ring.

As used herein, the term “alkenyl” refers to a group containing one or more carbon-carbon double bonds, which may be branched or unbranched, substituted (mono- or poly-) or unsubstituted. Preferably the alkenyl group is a C₂₋₂₀ alkenyl group, more preferably a C₂₋₁₅ alkenyl group, more preferably still a C₂₋₁₂ alkenyl group, or preferably a C₂₋₆ alkenyl group, more preferably a C₂₋₃ alkenyl group.

As used herein the phrase “preparation of a medicament” includes the use of a compound of formula I directly as the medicament in addition to its use in a screening programme for further anti-viral agents or in any stage of the manufacture of such a medicament.

Preferably, where R⁴⁻⁸ are each independently substituted lower alkyl, or substituted alkoxy, suitable substituents include, for example, one or more groups selected from halogeno, NO₂, CN, OH, O-methyl, NH₂, COOH, N—(R′)(R″), CONH₂ and CF₃.

Preferably, where R′ and R″ are each independently substituted lower alkyl, or substituted alkenyl, suitable substituents include, for example, one or more groups selected from halogeno, NO₂, CN, OH, O-methyl, NH₂, COOH, N—(R′)(R″), CONH₂ and CF₃.

Preferably, one of X¹ and X² is S, and the other of X¹ and X² is N, “a” is a single bond; “b”, “c”, “d”, “e” and “f” are single or double bonds so as to form a thiazolyl ring; R² is independently as defined above for R¹ and R³; R¹, R³ and R⁴⁻⁸ are as defined above.

In one preferred embodiment, the invention relates to the use of one or more compounds of formula Ia 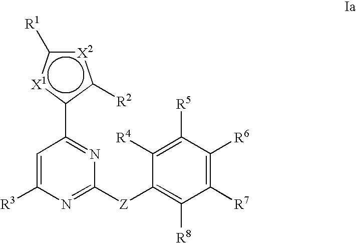
wherein:
one of X¹ and X² is S, and the other of X¹ and X² is N;
   Z is NH, NHCO, NHSO₂, NHCH₂, CH₂, CH₂CH₂, or CH═CH;

R¹, R², and R³ are independently H, alkyl, aryl, aralkyl, heterocycle, halogeno, NO₂, CN, OH, alkoxy, aryloxy, NH₂, NH-alkyl, N—(R′)(R″), NH-aryl, N-(aryl)₂, COOH, COO-alkyl, COO-aryl, CONH₂, CONH—R′, CON—(R′)(R″), CONH-aryl, CON-(aryl)₂, SO₃H, SO₂NH₂, CF₃, CO—R′, or CO-aryl, wherein said alkyl, NH-aryl, COO-alkyl, NH-alkyl, aryl, aralkyl and heterocycle groups may be further substituted with one or more groups selected from halogeno, NO₂, CN, OH, O-methyl, NH₂, COOH, N—(R′)(R″), CONH₂ and CF₃;

R⁴, R⁵, R⁶, R⁷, and R⁸ are independently from each other H, substituted or unsubstituted lower alkyl, halogeno, NO₂, CN, OH, substituted or unsubstituted alkoxy, NH₂, NH—R′, alkyl-aryl, alkyl-heteroaryl, NH(C═NH)NH₂, N(R′)₃⁺, N—(R′)(R″), COOH, COO—R′, CONH₂, CONH—R′, CON—(R′)(R″), SO₃H, SO₂NH₂, CF₃ or (CH₂)ₙO(CH₂)ₘNR′R″, (CH₂)ₙCO₂(CH₂)ₘOR′″ wherein n is 0, 1, 2 or 3 and m is 1, 2 or 3;

wherein R′ and R″ are each independently substituted or unsubstituted alkyl or alkenyl groups that may be the same or different; and pharmaceutically acceptable salts thereof; in the preparation of a medicament for use in the treatment of a viral disorder.

Thus, preferably, the compounds of formula I bear a mono- or di-substituted thiazol-3-yl or thiazol-5-yl radical attached to the pyrimidine ring through one of the ring carbon atoms Most preferably, the heterocycle is a thiazol-5-yl group.

In a preferred embodiment of the invention,
X¹ and X² are S and N respectively;
R¹, R² and R³ are each independently selected from H, alkyl, aryl, aralkyl, halogeno, NO₂, CN, OH, alkoxy, aryloxy, NH₂, NHCOR′, NHCOR′, NH-aryl, NH-alkyl, N—(R′)(R″), COOH, COO-alkyl, CONH₂, CONH—R′, CON—(R′)(R″), SO₃H, SO₂NH₂, CF₃, and CO—R′ wherein alkyl, aryl, COO-alkyl, NH-alkyl, NH-aryl and aralkyl groups may be further substituted with one or more groups selected from halogeno, NO₂, CN, OH, O-methyl, NH₂, COOH, CONH₂ and CF₃;
Z is selected from N, NHSO₂ and NHCH₂;
R⁴-R⁸ are each independently selected from H, OH, halogeno, nitro, amino, alkoxy, carbamoyl, sulfamyl, C₁₋₄ alkyl, substituted C₁₋₄ alkyl, SO₃H, COOH, COOR′, CN, CF₃, (CH₂)ₙO(CH₂)ₘNR′R″, alkyl-aryl, alkyl-heteroaryl, NH(C═NH)NH₂, N(R′)₃⁺, N(R′)(R″) and (CH₂)ₙCO₂(CH₂)ₘOR″′.

R′, R″, and R′″ are each independently preferably methyl or ethyl.

In yet another preferred embodiment Z is NH or NHSO₂.

More preferably, Z is NH.

In one particularly preferred embodiment, R¹ and R² are each independently one or more of halogen, a C₁₋₄ alkyl group, H, aryl, heterocycle, alkoxy, NH₂, NH-alkyl or N(R′)(R″).

In a more preferred embodiment, R¹ and R² are both methyl.

In one preferred embodiment, R³ is selected from H, aryl, substituted aryl, halo, C₁₋₄ alkoxy and OH. More preferably still, R³ is H.

In another preferred embodiment, R⁴ to R⁸ are selected independently from F, NH₂, NO₂, OH, Cl, Br, I, CF₃, OMe, COOH, COOR′, CN, H, C₁₋₄ alkyl, C₁₋₄ alkoxy, CH₂CO₂CH₂CH₂OMe, NH(C═NH)NH₂, CH₂CH₂OH, OCH₂CH₂NEt₂, SO₃H, N(Et)CH₂CH₂OH, CO₂CH₂CH₂OMe, CH₂OCH₂CH₂NEt₂, CH₂-heteroaryl, NMe₃⁺, and NMe₂.

In one especially preferred embodiment, the compound of formula I is selected from:
(a) 2-[N-(phenyl)]-4-(2,4-dimethylthiazol-5-yl)pyrimidineamines in which the phenyl group is 2-, 3- or 4-substituted by at least one of Me, F, NH₂, NO₂, OH, Cl, Br, I, CF₃, OMe, CN, COOH, CH₂OH, COOMe, COOEt, NH(C═NH)NH₂, CH₂CO₂CH₂CH₂OMe, CH₂-pyridyl, CH₂OCH₂CH₂NEt₂, CH₂CH₂OH, N(Et)CH₂CH₂OH, OCH₂CH₂NEt₂, CO₂CH₂CH₂OMe, NMe₃⁺ and NMe₂;
(b) 2-[N-(phenyl)]-4-(2-amino-4-methylthiazol-5-yl)pyrimidineamines in which the phenyl group is 2-, 3- or 4-substituted by at least one of NO₂, NH₂, Cl, CH₂CH₂OH, OMe, F, CF₃, I, Br, SO₃H, N(R′)R″), OH, or NH₂;
(c) 2-[N-(phenyl)]-4-(2-methoxy-4-methylthiazol-5-yl)pyrimidineamines in which the phenyl group is 2-, 3- or 4-substituted by at least one of N(R′)R″), OH, OMe, NO₂, Me, I, Cl or F; and
(d) 2-[N-(phenyl)]-4-(4-methyl-2-methylamino-thiazol-5-yl)pyrimidineamines or 2-[N-(phenyl)]-4-(4-methyl-2-ethylamino-thiazol-5-yl)pyrimidineamines in which the phenyl group is 2-, 3- or 4-substituted by at least one of F, N(R′)R″), Me, OH, I, NO₂, Cl, COOR′, Br, OMe or CF₃.

For each of the above groups (a) to (d), the preferred substituents are as follows:
for group (a) the phenyl group is mono-substituted by OCH₂CH₂NEt₂, CH₂CH₂OH, N(Et)CH₂CH₂OH, SO₃H, NMe₂, F, NH₂, NO₂, OH, Cl, Br, I, CF₃, OMe, CN, CH₂OH, COOH, COOMe, COOEt, CH₂CO₂CH₂CH₂OMe or CO₂CH₂CH₂OMe at any of the 2,3 or 4-positions, or di-substituted by 2,4-difluoro, 3,5-difluoro, 3,4-difluoro, 2,4-dichloro, 3,5-dichloro, 3,4-dichloro, 4-hydroxy-2-nitro, 4-hydroxy-3-nitro, 6-chloro-3-carboxy, 4-chloro-3-carboxy, 6-chloro-2-carboxy, 2-fluoro-4-iodo, 2-hydroxy-4-methoxy, 3-chloro-4-iodo, 3-chloro-4-hydroxy, 3-chloro-4-methyl, 3-chloro-4-methoxy, 4-fluoro-3-nitro, 6-chloro-3-methoxycarbonyl, 3-chloro-4-methoxcarbonyl, 3-chloro-4-ethoxcarbonyl, 3,4-dimethoxy, 3-hydroxy-4-methoxy, 4-dimethylamino-3-nitro, 2-chloro-5-methoxycarbonyl, 4-chloro-3-methoxycarbonyl, 6-chloro-3-(CO₂CH₂CH₂OMe), 3-chloro-4-(CO₂CH₂CH₂OMe), 4-chloro-3-trifluoromethyl, 3-chloro-4-dimethylamino, 3-dimethylamino-4-methoxy or 3-(CO₂CH₂CH₂OMe)-4-fluoro;
for group (b) the phenyl group is mono-substituted by NH₂, SO₃H, N(R′)(R″), OMe, F, Cl, Br, I, CH₂CH₂OH, nitro or OH at any of the 2,3 or 4-positions, or di-substituted by 4-iodo-3-nitro, 4-chloro-3-trifluoromethyl;
for group (c) the phenyl group is monosubstituted by NO₂, OH, I, F, Cl, OMe, N(R′)(R″) at any of the 2,3 or 4-positions, or di-substituted by 4-methyl-3-nitro, 4-fluoro-3-methyl, 3-iodo-4-methyl, 4-chloro-3-methyl, 4-iodo-3-nitro, 4-methly-3-nitro;
for group (d) the phenyl group is mono-substituted by chloro, bromo, iodo, fluoro, OH, nitro, CF₃ or OMe at any of the 2, 3 or 4 positions, or disubstituted by 4-hydroxy-3-nitro, 3-chloro-4-ethoxycarbonyl, 3,4-difluoro, 2,4-difluoro, 4-chloro-3-trifluoromethyl or 4-fluoro-3-nitro.

For group (a), in a particularly preferred embodiment, the phenyl group is monosubstituted by Br, I, NO₂, F, OMe, Cl, OH, CN or CF₃.

Another preferred embodiment of the invention, relates to the use of one or more compounds of formula Ib, or pharmaceutically acceptable salts thereof, 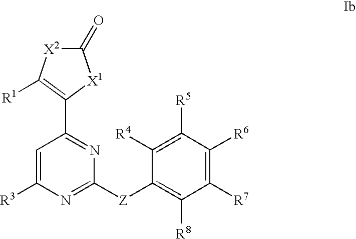
wherein one of X¹ and X² is S, and the other of X¹ and X² is NR⁹, and R¹⁻⁹ are as defined above, in the preparation of a medicament for treating a viral disorder.

Preferably, for this embodiment, X¹ is S, X² is NR⁹ and R⁹ is alkyl, preferably methyl.

In one especially preferred embodiment of the invention, said compound of formula I is selected from compounds [1]-[164] listed in Table 1.

In one particularly preferred embodiment, said compound of formula I is selected from the following:
[4-(2,4-Dimethyl-thiazol-5-yl)-pyrimidin-2-yl]-(3-nitro-phenyl)-amine [5];
3-[4-(2,4-Dimethyl-thiazol-5-yl)-pyrimidin-2-ylamino]-phenol [27];
4-[4-(2,4-Dimethyl-thiazol-5-yl)-pyrimidin-2-ylamino]-phenol [28]; and
4-[4-(2,4-Dimethyl-thiazol-5-yl)-pyrimidin-2-ylamino]-2-nitro-phenol [32];
N-[4-(2,4-Dimethyl-thiazol-5-yl)-pyrimidin-2-yl]-benzene-1,3-diamine [34];
(4-Fluoro-phenyl)-[4-(4-methyl-2-methylamino-thiazol-5-yl)-pyrimidin-2-yl]-amine [471;
4-[4-(4-Methyl-2-methylamino-thiazol-5-yl)-pyrimidin-2-ylamino]-phenol [48].
[4-(2-Ethylamino-4-methyl-thiazol-5-yl)-pyrimidin-2-yl]-(4-fluoro-phenyl)-amine [60];
[4-(4-Methyl-2-methylamino-thiazol-5-yl)-pyrimidin-2-yl]-(3-nitro-phenyl)-amine [61];
3-[4-(4-Methyl-2-methylamino-thiazol-5-yl)-pyrimidin-2-ylamino]-phenol [62];
(3-Methoxy-phenyl)-[4-(4-methyl-2-methylamino-thiazol-5-yl)-pyrimidin-2-yl]-amine [73];
N-[4-(2,4-Dimethyl-thiazol-5-yl)-pyrimidin-2-yl]-N′,N′-dimethyl-benzene-1,4-diamine [103];
[4-(2-Amino-4-methyl-thiazol-5-yl)-pyrimidin-2-yl]-(3-nitro-phenyl)-amine [105];
3-[4-(2-Amino-4-methyl-thiazol-5-yl)-pyrimidin-2-ylamino]-phenol [116];
(3,4-Dimethoxy-phenyl)-[4-(2,4-dimethyl-thiazol-5-yl)-pyrimidin-2-yl]-amine [125];
5-[4-(2,4-Dimethyl-thiazol-5-yl)-pyrimidin-2-ylamino]-2-methoxy-phenol [126];
N⁴-[4-(2,4-Dimethyl-thiazol-5-yl)-pyrimidin-2-yl]-N¹,N¹-dimethyl-2-nitro-benzene-1,4-diamine [127];
(4-Chloro-3-methyl-phenyl)-[4-(2-methoxy-4-methyl-thiazol-5-yl)-pyrimidin-2-yl]-amine [141];
(3-Iodo-4-methyl-phenyl)-[4-(2-methoxy-4-methyl-thiazol-5-yl)-pyrimidin-2-yl]-amine [142];
(4-Fluoro-3-methyl-phenyl)-[4-(2-methoxy-4-methyl-thiazol-5-yl)-pyrimidin-2-yl]-amine [143];
[4-(2-Methoxy-4-methyl-thiazol-5-yl)-pyrimidin-2-yl]-(4-methyl-3-nitro-phenyl)-amine [144];
[4-(2-Ethylamino-4-methyl-thiazol-5-yl)-pyrimidin-2-yl]-(3-nitro-phenyl)-amine [133]
N-[4-(2-Amino-4-methyl-thiazol-5-yl)-pyrimidin-2-yl]-benzene-1,3-diamine [149];
[4-(2,4-Dimethyl-thiazol-5-yl)-pyrimidin-2-yl]-phenyl-amine [150].

In a more preferred embodiment, said compound of formula I is capable of inhibiting CDK2 and/or CDK7 and/or CDK9 and is selected from the following:
(4-Chloro-phenyl)-[4-(2,4-dimethyl-thiazol-5-yl)-pyrimidin-2-yl]-amine [2];
(3-Chloro-phenyl)-[4-(2,4-dimethyl-thiazol-5-yl)-pyrimidin-2-yl]-amine [3];
[4-(2,4-Dimethyl-thiazol-5-yl)-pyrimidin-2-yl]-(3-nitro-phenyl)-amine [5];
[4-(2,4-Dimethyl-thiazol-5-yl)-pyrimidin-2-yl]-(4-nitro-phenyl)-amine [6];
[4-(2,4-Dimethyl-thiazol-5-yl)-pyrimidin-2-yl]-(2-fluoro-phenyl)-amine [7];
[4-(2,4-Dimethyl-thiazol-5-yl)-pyrimidin-2-yl]-(4-fluoro-phenyl)-amine [8];
(2,4-Difluoro-phenyl)-[4-(2,4-dimethyl-thiazol-5-yl)-pyrimidin-2-yl]-amine [9];
(3,5-Difluoro-phenyl)-[4-(2,4-dimethyl-thiazol-5-yl)-pyrimidin-2-yl]-amine [10];
(3,5-Dichloro-phenyl)-[4-(2,4-dimethyl-thiazol-5-yl)-pyrimidin-2-yl]-amine [11];
(2,4-Dichloro-phenyl)-[4-(2,4-dimethyl-thiazol-5-yl)-pyrimidin-2-yl]-amine [12];
[4-(2,4-Dimethyl-thiazol-5-yl)-pyrimidin-2-yl]-(4-trifluoromethyl-phenyl)-amine [15];
(3-Bromo-phenyl)-[4-(2,4-dimethyl-thiazol-5-yl)-pyrimidin-2-yl]-amine [17];
[4-(2,4-Dimethyl-thiazol-5-yl)-pyrimidin-2-yl]-(3-iodo-phenyl)-amine [20];
[4-(2,4-Dimethyl-thiazol-5-yl)-pyrimidin-2-yl]-(3-fluoro-phenyl)-amine [22];
(3,4-Difluoro-phenyl)-[4-(2,4-dimethyl-thiazol-5-yl)-pyrimidin-2-yl]-amine [23];
[4-(2,4-Dimethyl-thiazol-5-yl)-pyrimidin-2-yl]-(2-methoxy-phenyl)-amine [24];
[4-(2,4-Dimethyl-thiazol-5-yl)-pyrimidin-2-yl]-(3-methoxy-phenyl)-amine [25];
[4-(2,4-Dimethyl-thiazol-5-yl)-pyrimidin-2-yl]-(4-methoxy-phenyl)-amine [26];
3-[4-(2,4-Dimethyl-thiazol-5-yl)-pyrimidin-2-ylamino]-phenol [27];
4-[4-(2,4-Dimethyl-thiazol-5-yl)-pyrimidin-2-ylamino]-phenol [28];
4-[4-(2,4-Dimethyl-thiazol-5-yl)-pyrimidin-2-ylamino]-2-nitro-phenol [32];
N-[4-(2,4-Dimethyl-thiazol-5-yl)-pyrimidin-2-yl]-benzene-1,3-diamine [34];
4-[4-(2,4-Dimethyl-thiazol-5-yl)-pyrimidin-2-ylamino]-benzonitrile [35];
3-[4-(2,4-Dimethyl-thiazol-5-yl)-pyrimidin-2-ylamino]-benzonitrile [36];
4-[4-(2,4-Dimethyl-thiazol-5-yl)-pyrimidin-2-ylamino]-benzoic acid methyl ester [37];
(3-Chloro-4-methoxy-phenyl)-[4-(2,4-dimethyl-thiazol-5-yl)-pyrimidin-2-yl]-amine [39];
4-[4-(2,4-Dimethyl-thiazol-5-yl)-pyrimidin-2-ylamino]-benzoic acid [40];
[4-Bromo-6-(2,4-dimethyl-thiazol-5-yl)-pyrimidin-2-yl]-(3-nitro-phenyl)-amine [41];
(4-Fluoro-phenyl)-[4-(4-methyl-2-methylamino-thiazol-5-yl)-pyrimidin-2-yl]-amine [47];
4-[4-(4-Methyl-2-methylamino-thiazol-5-yl)-pyrimidin-2-ylamino]-phenol [48];
4-[4-(4-Methyl-2-methylamino-thiazol-5-yl)-pyrimidin-2-ylamino]-2-nitro-phenol [58];
[4-(2-Ethylamino-4-methyl-thiazol-5-yl)-pyrimidin-2-yl]-(4-fluoro-phenyl)-amine [60];
[4-(4-Methyl-2-methylamino-thiazol-5-yl)-pyrimidin-2-yl]-(3-nitro-phenyl)-amine [61];
[4-(2-Allylamino-4-methyl-thiazol-5-yl)-pyrimidin-2-yl]-(4-fluoro-phenyl)-amine [67];
(3-Bromo-phenyl)-[4-(4-methyl-2-methylamino-thiazol-5-yl)-pyrimidin-2-yl]-amine [68];
[4-(2-Allylamino-4-methyl-thiazol-5-yl)-pyrimidin-2-yl]-(3-nitro-phenyl)-amine [69];
3-[4-(4-Methyl-2-methylamino-thiazol-5-yl)-pyrimidin-2-ylamino]-phenol [70];
(4-Chloro-phenyl)-[4-(4-methyl-2-methylamino-thiazol-5-yl)-pyrimidin-2-yl]-amine [72];
(3-Methoxy-phenyl)-[4-(4-methyl-2-methylamino-thiazol-5-yl)-pyrimidin-2-yl]-amine [73];
[4-(4-Methyl-2-methylamino-thiazol-5-yl)-pyrimidin-2-yl]-(4-trifluoromethyl-phenyl)-amine [74];
[4-(4-Methyl-2-methylamino -thiazol- 5-yl) -pyrimidin-2-yl]-(3-trifluoromethyl-phenyl)-amine [75];
2-[4-(2,4-Dimethyl-thiazol-5-yl)-pyrimidin-2-ylamino]-5-methoxy-phenol [79];
2-Chloro-5-[4-(2,4-dimethyl-thiazol-5-yl)-pyrimidin-2-ylamino]-benzoic acid methyl ester; [83];
(3-Fluoro-phenyl)-[4-(4-methyl-2-methylamino-thiazol-5-yl)-pyrimidin-2-yl]-amine [87];
(4-Methoxy-phenyl)-[4-(4-methyl-2-methylamino-thiazol-5-yl)-pyrimidin-2-yl]-amine [93];
4-{4-[2-(4-Nitro-phenylamino)-thiazol-5-yl]-pyrimidin-2-ylamino}-phenol [95];
4-[4-(2-Amino-4-methyl-thiazol-5-yl)-pyrimidin-2-ylamino]-phenol [98];
N-{3-[4-(2,4-Dimethyl-thiazol-5-yl)-pyrimidin-2-ylamino]-phenyl}-guanidine [99];
{3-[4-(2,4-Dimethyl-thiazol-5-yl)-pyrimidin-2-ylamino]-phenyl}-methanol [100];
[4-(2,4-Dimethyl-thiazol-5-yl)-pyrimidin-2-yl]-(4-pyridin-4-ylmethyl-phenyl)-amine [101];
N,N-Dimethyl-N′-[4-(2,4-dimethyl-thiazol-5-yl)-pyrimidin-2-yl]-benzene-1,4-diamine [103];
{4-[4-(2,4-Dimethyl-thiazol-5-yl)-pyrimidin-2-ylamino]-phenyl}-trimethyl-ammonium [104];
[4-(2-Amino-4-methyl-thiazol-5-yl)-pyrimidin-2-yl]-(3-nitro-phenyl)-amine [105];
N-[4-(2-Amino-4-methyl-thiazol-5-yl)-pyrimidin-2-yl]-N′,N′-dimethyl-benzene-1,4-diamine [106];
[4-(2-Amino-4-methyl-thiazol-[-yl)-pyrimidin-2-yl]-(3-methoxy-phenyl)-amine [108];
[4-(2-Amino-4-methyl-thiazol-5-yl)-pyrimidin-2-yl]-(3-fluoro-phenyl)-amine [109];
[4-(2-Amino-4-methyl-thiazol-5-yl)-pyrimidin-2-yl]-(4-trifluoromethyl-phenyl)-amine [110];
[4-(2-Amino-4-methyl-thiazol-5-yl)-pyrimidin-2-yl]-(4-methoxy-phenyl)-amine [111];
[4-(2-Amino-4-methyl-thiazol-5-yl)-pyrimidin-2-yl]-(3-chloro-phenyl)-amine [112];
[4-(2-Amino-4-methyl-thiazol-5-yl)-pyrimidin-2-yl]-(3-iodo-phenyl)-amine [113];
3-[4-(2-Amino-4-methyl-thiazol-5-yl)-pyrimidin-2-ylamino]-phenol [116];
[4-(2-Amino-4-methyl-thiazol-5-yl)-pyrimidin-2-yl]-(4-iodo-3-nitro-phenyl)-amine [117];
2-{4-[4-(2-Amino-4-methyl-thiazol-5-yl)-pyrimidin-2-ylamino]-phenyl}-ethanol [118];
[4-(2-Amino-4-methyl-thiazol-5-yl)-pyrimidin-2-yl]-(3-bromo-phenyl)-amine [119];
[4-(2-Amino-4-methyl-thiazol-5-yl)-pyrimidin-2-yl]-(4-bromo-phenyl)-amine [120];
N¹-[4-(2,4-Dimethyl-thiazol-5-yl)-pyrimidin-2-yl]-[β-(phenoxy)-triethylamine]-amine [122];
2-{4-[4-(2,4-Dimethyl-thiazol-5-yl)-pyrimidin-2-ylamino]-phenyl}-ethanol [123];
2-({4-[4-(2,4-Dimethyl-thiazol-5-yl)-pyrimidin-2-ylamino]-phenyl}-ethyl-amino)-ethanol [124];
(3,4-Dimethoxy-phenyl)-[4-(2,4-dimethyl-thiazol-5-yl)-pyrimidin-2-yl]-amine [125];
5-[4-(2,4-Dimethyl-thiazol-5-yl)-pyrimidin-2-ylamino]-2-methoxy-phenol [126];
N⁴-[4-(2,4-Dimethyl-thiazol-5-yl)-pyrimidin-2-yl]-N¹,N¹-dimethyl-2-nitro-benzene-1,4-diamine [127];
2-[N-(4-N,N-Dimethylamino-3-chlorophenyl)]-4-(2,4-dimethylthiazol-5-yl)-pyrimidineamine [128];
N¹-[4-(2,4-Dimethyl-thiazol-5-yl)-pyrimidin-2-yl]-4-methoxy-N³,N³-dimethyl-benzene-1,3-diamine [130];
N,N-Dimethyl-N′-[4-(4-methyl-2-methylamino-thiazol-1-yl)-pyrimidin-2-yl]-benzene-1,4-diamine [131];
(4-Iodo-3-nitro-phenyl)-[4-(4-methyl-2-methylamino-thiazol-5-yl)-pyrimidin-2-yl]-amine [132];
[4-(2-Ethylamino-4-methyl-thiazol-5-yl)-pyrimidin-2-yl]-(3-nitro-phenyl)-amine [133]
(4-Chloro-phenyl)-[4-(2-ethylamino-4-methyl-thiazol-5-yl)-pyrimidin-2-yl]-amine [134];
[4-(2-Ethylamino-4-methyl-thiazol-5-yl)-pyrimidin-2-yl]-(3-methoxy-phenyl)-amine [136];
[4-(2-Ethylamino-4-methyl-thiazol-5-yl)-pyrimidin-2-yl]-(4-methyl-3-nitro-phenyl)-amine [138];
[4-(2-Butylamino-4-methyl-thiazol-5-yl)-pyrimidin-2-yl]-(4-fluoro-phenyl)-amine [139];
[4-(2-Dimethylamino-4-methyl-thiazol-5-yl)-pyrimidin-2-yl]-(3-nitro-phenyl)-amine [140];
(4-Chloro-phenyl)-[4-(2-dimethylamino-4-methyl-thiazol-5-yl)-pyrimidin-2-yl]-amine [141];
[4-(2-Dimethylamino-4-methyl-thiazol-5-yl)-pyrimidin-2-yl]-(4-fluoro-phenyl)-amine [142];
(3-Chloro-phenyl)-[4-(2-dimethylamino-4-methyl-thiazol-5-yl)-pyrimidin-2-yl]-amine [143];
2-{4-Methyl-5-[2-(3-nitro-phenylamino)-pyrimidin-4-yl]-thiazol-2-ylamino}-ethanol [144];
2-{5-[2-(4-Fluoro-phenylamino)-pyrimidin-4-yl]-4-methyl-thiazol-2-ylamino}-ethanol [145];
4-[4-(2,4-Dimethyl-thiazol-5-yl)-pyrimidin-2-ylamino]-benzenesulfonic acid [148];
N-[4-(2-Amino-4-methyl-thiazol-5-yl)-pyrimidin-2-yl]-benzene- 1,3-diamine [149].
[4-(2,4-Dimethyl-thiazol-5-yl)-pyrimidin-2-yl]-phenyl-amine [150]; and [4-(2-Amino-4-methyl-thiazol-5-yl)-pyrimidin-2-yl]-(4-nitro-phenyl)-amine [151].

In a still further preferred embodiment, said compound of formula I is capable of inhibiting CDK2 and/or CDK7 and/or CDK9 and is selected from the following:
[4-(2,4-Dimethyl-thiazol-5-yl)-pyrimidin-2-yl]-(3-nitro-phenyl)-amine [5];
[4-(2,4-Dimethyl-thiazol-5-yl)-pyrimidin-2-yl]-(4-fluoro-phenyl)-amine [8];
(2,4-Difluoro-phenyl)-[4-(2,4-dimethyl-thiazol-5-yl)-pyrimidin-2-yl]-amine [9];
[4-(2,4-Dimethyl-thiazol-5-yl)-pyrimidin-2-yl]-(4-trifluoromethyl-phenyl)-amine [15];
(3-Bromo-phenyl)-[4-(2,4-dimethyl-thiazol-5-yl)-pyrimidin-2-yl]-amine [17];
[4-(2,4-Dimethyl-thiazol-5-yl)-pyrimidin-2-yl]-(3-fluoro-phenyl)-amine [22];
(3,4-Difluoro-phenyl)-[4-(2,4-dimethyl-thiazol-5-yl)-pyrimidin-2-yl]-amine [23];
[4-(2,4-Dimethyl-thiazol-5-yl)-pyrimidin-2-yl]-(3-methoxy-phenyl)-amine [25];
[4-(2,4-Dimethyl-thiazol-5-yl)-pyrimidin-2-yl]-(4-methoxy-phenyl)-amine [26];
3-[4-(2,4-Dimethyl-thiazol-5-yl)-pyrimidin-2-ylamino]-phenol [27];
4-[4-(2,4-Dimethyl-thiazol-5-yl)-pyrimidin-2-ylamino]-phenol [28];
4-[4-(2,4-Dimethyl-thiazol-5-yl)-pyrimidin-2-ylamino]-2-nitro-phenol [32];
N-[4-(2,4-Dimethyl-thiazol-5-yl)-pyrimidin-2-yl]-benzene-1,3-diamine [34];
3-[4-(2,4-Dimethyl-thiazol-5-yl)-pyrimidin-2-ylamino]-benzonitrile [36];
4-[4-(2,4-Dimethyl-thiazol-5-yl)-pyrimidin-2-ylamino]-benzoic acid [40];
[4-Bromo-6-(2,4-dimethyl-thiazol-5-yl)-pyrimidin-2-yl]-(3-nitro-phenyl)-amine [41];
(4-Fluoro-phenyl)-[4-(4-methyl-2-methylamino-thiazol-5-yl)-pyrimidin-2-yl]-amine [47];
4-[4-(4-Methyl-2-methylamino-thiazol-5-yl)-pyrimidin-2-ylamino]-phenol [48];
4-[4-(4-Methyl-2-methylamino-thiazol-5-yl)-pyrimidin-2-ylamino]-2-nitro-phenol [58];
[4-(2-Ethylamino-4-methyl-thiazol-5-yl)-pyrimidin-2-yl]-(4-fluoro-phenyl)-amine [60];
[4-(4-Methyl-2-methylamino-thiazol-5-yl)-pyrimidin-2-yl]-(3-nitro-phenyl)-amine [61];
(3-Bromo-phenyl)-[4-(4-methyl-2-methylamino-thiazol-5-yl)-pyrimidin-2-yl]-amine [68];
[4-(2-Allylamino-4-methyl-thiazol-5-yl)-pyrimidin-2-yl]-(3-nitro-phenyl)-amine [69];
3-[4-(4-Methyl-2-methylamino-thiazol-5-yl)-pyrimidin-2-ylamino]-phenol [70];
(3-Methoxy-phenyl)-[4-(4-methyl-2-methylamino-thiazol-5-yl)-pyrimidin-2-yl]-amine [73];
[4-(4-Methyl-2-methylamino-thiazol-5-yl)-pyrimidin-2-yl]-(3-trifluoromethyl-phenyl)-amine [75];
(3-Fluoro-phenyl)-[4-(4-methyl-2-methylamino-thiazol-5-yl)-pyrimidin-2-yl]-amine [87];
(4-Methoxy-phenyl)-[4-(4-methyl-2-methylamino-thiazol-5-yl)-pyrimidin-2-yl]-amine [93];
4-{4-[2-(4-Nitro-phenylamino)-thiazol-5-yl]-pyrimidin-2-ylamino}-phenol [95];
4-[4-(2-Amino-4-methyl-thiazol-5-yl)-pyrimidin-2-ylamino]-phenol [98];
N-{3-[4-(2,4-Dimethyl-thiazol-5-yl)-pyrimidin-2-ylamino]-phenyl}-guanidine [99];
{3-[4-(2,4-Dimethyl-thiazol-5-yl)-pyrimidin-2-ylamino]-phenyl}-methanol [100];
[4-(2,4-Dimethyl-thiazol-5-yl)-pyrimidin-2-yl]-(4-pyridin-4-ylmethyl-phenyl)-a-mine [101];
N,N-Dimethyl-N′-[4-(2,4-dimethyl-thiazol-5-yl)-pyrimidin-2-yl]-benzene-1,4-diamine [103];
{4-[4-(2,4-Dimethyl-thiazol-5-yl)-pyrimidin-2-ylamino]-phenyl}-trimethyl-ammonium 104];
[4-(2-Amino-4-methyl-thiazol-1-yl)-pyrimidin-2-yl]-(3-nitro-phenyl)-amine [105];
N-[4-(2-Amino-4-methyl-thiazol-5-yl)-pyrimidin-2-yl]-N′,N′-dimethyl-benzene-1,4-diamine [106];
[4-(2-Amino-4-methyl-thiazol-5-yl)-pyrimidin-2-yl]-(3-methoxy-phenyl)-amine [108];
[4-(2-Amino-4-methyl-thiazol-5-yl)-pyrimidin-2-yl]-(3-fluoro-phenyl)-amine [109];
[4-(2-Amino-4-methyl-thiazol-5-yl)-pyrimidin-2-yl]-(4-trifluoromethyl-phenyl)-amine [110];
[4-(2-Amino-4-methyl-thiazol-5-yl)-pyrimidin-2-yl]-(4-methoxy-phenyl)-amine [111];
[4-(2-Amino-4-methyl-thiazol-5-yl)-pyrimidin-2-yl]-(3-chloro-phenyl)-amine [112];
[4-(2-Amino-4-methyl-thiazol-5-yl)-pyrimidin-2-yl]-(3-iodo-phenyl)-amine [113];
3-[4-(2-Amino-4-methyl-thiazol-5-yl)-pyrimidin-2-ylamino]-phenol [116];
[4-(2-Amino-4-methyl-thiazol-5-yl)-pyrimidin-2-yl]-(4-iodo-3-nitro-phenyl)-amine [117];
2-{4-[4-(2-Amino-4-methyl-thiazol-5-yl)-pyrimidin-2-ylamino]-phenyl}-ethanol [118];
[4-(2-Amino-4-methyl-thiazol-5-yl)-pyrimidin-2-yl]-(3-bromo-phenyl)-amine [119];
N¹-[4-(2,4-Dimethyl-thiazol-5-yl)-pyrimidin-2-yl]-4-[β-(phenoxy)-triethylamine]-amine [122];
2-{4-[4-(2,4-Dimethyl-thiazol-5-yl)-pyrimidin-2-ylamino]-phenyl}-ethanol [123];
(3,4-Dimethoxy-phenyl)-[4-(2,4-dimethyl-thiazol-5-yl)-pyrimidin-2-yl]-amine [125];
5-[4-(2,4-Dimethyl-thiazol-5-yl)-pyrimidin-2-ylamino]-2-methoxy-phenol [126];
N⁴-[4-(2,4-Dimethyl-thiazol-5-yl)-pyrimidin-2-yl]-N¹,N¹-dimethyl-2-nitro-benzene-1,4-diamine [127];
2-[N-(4-N,N-Dimethylamino-3-chlorophenyl)]-4-(2,4-dimethylthiazol-5-yl)-pyrimidineamine [128];
N¹-[4-(2,4-Dimethyl-thiazol-5-yl)-pyrimidin-2-yl]-4-methoxy-N³,N³-dimethyl-benzene-1,3-diamine [130];
N,N-Dimethyl-N′-[4-(4-methyl-2-methylamino-thiazol-5-yl)-pyrimidin-2-yl]-benzene-1,4-diamine [131];
(4-Iodo-3-nitro-phenyl)-[4-(4-methyl-2-methylamino-thiazol-5-yl)-pyrimidin-2-yl]-amine [132];
[4-(2-Ethylamino-4-methyl-thiazol-5-yl)-pyrimidin-2-yl]-(3-nitro-phenyl)-amine [133]
[4-(2-Dimethylamino-4-methyl-thiazol-5-yl)-pyrimidin-2-yl]-(3-nitro-phenyl)-amine [140];
(3-Chloro-phenyl)-[4-(2-dimethylamino-4-methyl-thiazol-5-yl)-pyrimidin-2-yl]-amine [143];
2-{4-Methyl-5-[2-(3-nitro-phenylamino)-pyrimidin-4-yl]-thiazol-2-ylamino}-ethanol [144];
2-{5-[2-(4-Fluoro-phenylamino)-pyrimidin-4-yl]-4-methyl-thiazol-2-ylamino}-ethanol [145];
4-[4-(2,4-Dimethyl-thiazol-5-yl)-pyrimidin-2-ylamino]-benzenesulfonic acid [148];
N-[4-(2-Amino-4-methyl-thiazol-5-yl)-pyrimidin-2-yl]-benzene-1,3-diamine [149].
[4-(2,4-Dimethyl-thiazol-5-yl)-pyrimidin-2-yl]-phenyl-amine [150];
[4-(2-Amino-4-methyl-thiazol-5-yl)-pyrimidin-2-yl]-(4-nitro-phenyl)-amine [551].

The following compounds are observed to be particularly effective anti-viral agents, as demonstrated by cell-based assays:
[4-(2,4-Dimethyl-thiazol-5-yl)-pyrimidin-2-yl]-(4-iodo-phenyl)-amine [21];
4-[4-(2,4-Dimethyl-thiazol-5-yl)-pyrimidin-2-ylamino]-phenol 128];
(4-Fluoro-phenyl)-[4-(4-methyl-2-methylamino-thiazol-5-yl)-pyrimidin-2-yl]-amine [47];
3-[4-(4-Methyl-2-methylamino-thiazol-5-yl)-pyrimidin-2-ylamino]-phenol [70];
N,N-Dimethyl-N′-[4-(2,4-dimethyl-thiazol-5-yl)-pyrimidin-2-yl]-benzene-1,4-diamine [103];
[4-(2-Amino-4-methyl-thiazol-5-yl)-pyrimidin-2-yl]-(3-nitro-phenyl)-amine [105];
(3,4-Dimethoxy-phenyl)-[4-(2,4-dimethyl-thiazol-5-yl)-pyrimidin-2-yl]-amine [125];
2-[N-(4-N,N-Dimethylamino-3-chlorophenyl)]-4-(2,4-dimethylthiazol-5-yl)-pyrimidineamine [128].

More preferably still, the compound is selected from the following:
[4-(2,4-Dimethyl-thiazol-5-yl)-pyrimidin-2-yl]-(4-iodo-phenyl)-amine [21];
4-[4-(2,4-Dimethyl-thiazol-5-yl)-pyrimidin-2-ylamino]-phenol [28];
(4-Fluoro-phenyl)-[4-(4-methyl-2-methylamino-thiazol-5-yl)-pyrimidin-2-yl]-amine [47];
N,N-Dimethyl-N′-[4-(2,4-dimethyl-thiazol-5-yl)-pyrimidin-2-yl]-benzene-1,4-diamine [103];
[4-(2-Amino-4-methyl-thiazol-5-yl)-pyrimidin-2-yl]-(3-nitro-phenyl)-amine [105];
(3,4-Dimethoxy-phenyl)-[4-(2,4-dimethyl-thiazol-5-yl)-pyrimidin-2-yl]-amine [125];
2-[N-(4-N,N-Dimethylamino-3-chlorophenyl)]-4-(2,4-dimethylthiazol-5-yl)-pyrimidineamine [128].

A further aspect of the invention relates to the use of a compound of formula I as defined hereinabove in the treatment of a viral disorder.

Therapeutic Applications

The compounds of the invention may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of the invention may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In one embodiment of the invention, the compound of formula I is administered in an amount sufficient to inhibit at least one CDK enzyme.

In a more preferred embodiment of the invention, the compound of formula I is preferably administered in an amount sufficient to inhibit one or more of the host cell CDKs involved in viral replication, i.e. CDK2, CDK7, CDK8, and CDK9 [Wang D, De la Fuente C, Deng L, Wang L, Zilberman I, Eadie C, Healey M, Stein D, Denny T, Harrison L E, Meijer L, Kashanchi F. Inhibition of human immunodeficiency virus type 1 transcription by chemical cyclin-dependent kinase inhibitors. J. Virol. 2001; 75: 7266-7279].

As defined herein, an anti-viral effect within the scope of the present invention may be demonstrated by the ability to inhibit CDK2, CDK7, CDK8 or CDK9. Assays for determining CDK activity are described in more detail in the accompanying examples. Using such enzymes assays it may be determined whether a compound is anti-viral in the context of the present invention.

In a particularly preferred embodiment, the compounds of the present invention are useful in the treatment of viral disorders, such as human cytomegalovirus (HCMV), herpes simplex virus type 1 (HSV-1), human immunodeficiency virus type 1 (HIV-1), and varicella zoster virus (VZV).

In a particularly preferred embodiment, the invention relates to the use of one or more compounds of formula I in the treatment of a viral disorder which is CDK dependent or sensitive. CDK dependent disorders are associated with an above normal level of activity of one or more CDK enzymes. Such disorders preferably associated with an abnormal level of activity of CDK2, CDK7, CDK8 and/or CDK9. A CDK sensitive disorder is a disorder in which an aberration in the CDK level is not the primary cause, but is downstream of the primary metabolic aberration. In such scenarios, CDK2, CDK7, CDK8 and/or CDK9 can be said to be part of the sensitive metabolic pathway and CDK inhibitors may therefore be active in treating such disorders.

In one preferred embodiment the compound of formula I is capable of exibiting an antiviral effect in human cell lines, as measured by an HIV-1 assay in human peripheral blood mononuclear cells. Preferably, the compound of formula I exihibits an IC₅₀ value of less than 10 μM, more preferably less than 5 μM, even more preferably less than 1 μM as measured by said MTT assay. More preferably still, the compound exihibits an IC₅₀ value of less than 0.5 μM, more preferably still less than 0.1 μM. More preferably still, the compound exibits an IC₅₀ value of less than 0.01 μM.

In one preferred embodiment, the compound of formula I is capable of inhibiting one or more CDKs associated with viral disorders.

In another preferred embodiment, the compound of formula I is capable of inhibiting one or more of CDK2, CDK7, CDK8 and CDK9, as measured by the assays described in the accompanying Examples section. Preferably, the compound of formula I exihibits an IC₅₀. value of less than 10 μM, more preferably less than 5 μM, even more preferably less than 1 μM or less than 0.5 less μM, more preferably still less than 0.1 μM. More preferably still, the compound exibits an IC₅₀ value of less than 0.01 μM.

Salts/Esters

The compounds used in the present invention can be present as salts or esters, in particular pharmaceutically acceptable salts or esters.

Pharmaceutically acceptable salts of the compounds of the invention (first and seconds aspects) include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers and Tautomers

The invention further includes, where appropriate, the use of all enantiomers and tautomers of compounds of formula I. The man skilled in the art will recognise compounds that possess an optical properties (one or more chiral carbon atoms) or tautomeric characteristics. The corresponding enantiomers and/or tautomers may be isolated/prepared by methods known in the art.

Polymorphs

The invention furthermore relates to the compounds of use in the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes the compounds of use in the present invention in prodrug form. Such prodrugs are generally compounds of formula I wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Pharmaceutical Compositions

In a preferred embodiment of the invention, the compound of formula I is administered in combination with a pharmaceutically acceptable excipient, diluent or carrier. In this regard, and in particular for human therapy, even though the compounds of the present invention (including their pharmaceutically acceptable salts, esters and pharmaceutically acceptable solvates) can be administered alone, they will generally be administered in admixture with a pharmaceutical carrier, excipient or diluent selected with regard to the intended route of administration and standard pharmaceutical practice.

Thus, the present invention also relates to the use of pharmaceutical compositions comprising one or more compounds of formula I or pharmaceutically acceptable salts or esters thereof, together with at least one pharmaceutically acceptable excipient, diluent or carrier.

By way of example, in the pharmaceutical compositions of the present invention, the compounds of the invention may be admixed with any suitable binder(s), lubricant(s), suspending agent(s), coating agent(s), and/or solubilising agent(s). Examples of such suitable excipients for the various different forms of pharmaceutical compositions described herein may be found in the “Handbook of Pharmaceutical Excipients, 2^{nd} Edition, (1994), Edited by A Wade and P J Weller.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

Dosages

A person of ordinary skill in the art can easily determine an appropriate dose of one of the instant compositions to administer to a subject without undue experimentation. Typically, a physician will determine the actual dosage which will be most suitable for an individual patient and it will vary with the age, weight and response of the particular patient. The dosages disclosed herein are exemplary of the average case. There can of course be individual instances where higher or lower dosage ranges are merited, and such are within the scope of this invention.

In an exemplary embodiment, one or more doses of 10 to 150 mg/day will be administered to the patient for the treatment of a viral disorder.

Combinations

In a particularly preferred embodiment, the one or more compounds of the invention are administered in combination with one or more other antiviral agents. In such cases, the compounds of the invention may be administered consecutively, simultaneously or sequentially with the one or more other antiviral agents.

It is known in the art that many drugs are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining drugs are that it may promote additive or possible synergistic effects through biochemical interactions and also may decrease the emergence of drug resistance which would have been otherwise responsive to initial treatment with a single agent.

Beneficial combinations may be suggested by studying the antiviral activity of the test compounds with agents known or suspected of being valuable in the treatment of a particular viral disorder. This procedure can also be used to determine the order of administration of the agents, i.e. before, simultaneously, or after delivery.

Chemical Synthesis

The compounds of this invention (I) can be synthesised, for example, by an adaptation of the Traube synthesis (A. R. Katritzky, T. I. Yousaf, Can. J. Chem. 1986, 64, 2087 and references cited therein), i.e. by condensation between 1,3-dicarbonyl compounds 1 or acrylates 2 or 3, and amidine 4, as shown in Scheme 1.

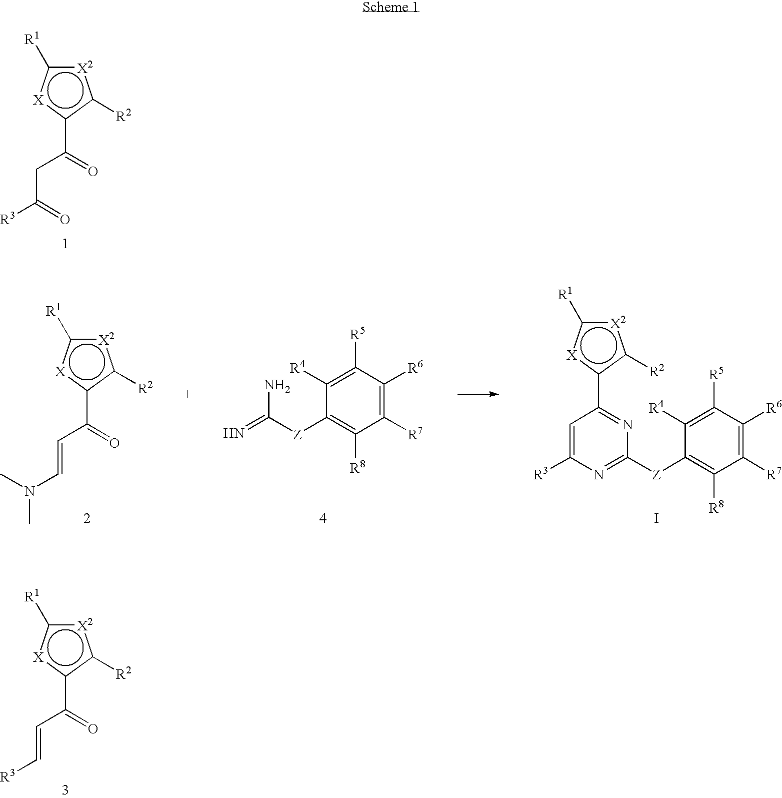

The dicarbonyl compounds 1 in turn can be prepared by many methods known in the art (J. March, In: Advanced Organic Chemistry: Reactions, Mechanism, and Structure, 4^{th} Ed., John Wiley & Sons, Inc., New York, 1992, p. 1283). Acrylates 2 and 3, which are particularly suitable for the purposes of this invention, are obtained from heterocyclic methyl ketones 5 by condensation with dimethylformamide dimethylacetal 6 and aldehydes 7 respectively, (Scheme 2).

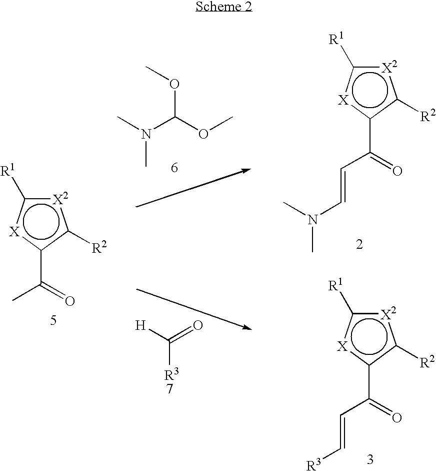

The diamino compounds 4 will be amidines 4a or guanidines 4b, depending on the definition of Z in general structure I. Amidines (HN═CRNH₂) can be obtained from readily available amine precursors by condensation with e.g. ketenimines, or by addition of ammonia to suitable nitrites or imidates. Guanidines 4b (Scheme 3) can be elaborated by a number of methods known in the art. For the purposes of this invention, the most useful route is amination of cyanamide 8 with anilines 9.

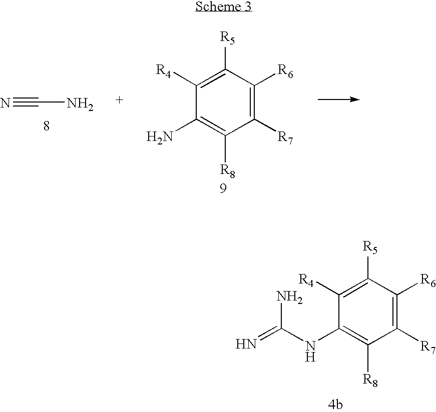

Alternatively, compounds of general structure I can be obtained from suitable pyrimidine precursors directly, e.g. from 2,4-disubstituted (halogen, amine, etc.) pyrimidines by successive substitution reactions.

The present invention is further described by way of example, with reference to the chemical structure of compounds [1]-[164] according to the invention.

### EXAMPLES

Abbreviations

DE MALDI-TOF MS, delayed extraction matrix assisted laser desorption ionisation time-of-flight mass spectrometry; DMF, N,N-dimethylformamide; LC-MS, liquid chromatography-mass spectrometry; NMR, nuclear magnetic resonance spectroscopy; RP-HPLC, reversed-phase high performance liquid chromatography; r.t. room temperature; PE, petroleum ether (40-60° C. boiling fraction); DMSO, dimethylsulfoxide.

General

NMR spectra were recorded using a Bruker DPX-300 instrument. Chemical shifts are reported in ppm (6) from tetramethylsilane. EM Kieselgel 60 (0.040-0.063 mm) was used for flash column chromatography. Melting points were determined with a LEICA testo-720 electrothermometer and are uncorrected. Compound numbers are shown in brackets, where appropriate.

### Example 1

3-Dimethylamino-1-(2,4-dimethyl-thiazol-5-yl)-propenone. A solution of 5-acetyl-2,4-dimethylthiazole (10 g, 60 mmol) in N,N-dimethylformamide dimethylacetal (10 mL) was refluxed under N₂. After 18 h, the reaction mixture was evaporated to dryness and the residue was recrystallised from iPr₂O/CH₂Cl₂ to afford the title compound as a brown powder (9.94 g, 79%). ¹H-NMR (300 MHz, CDCl₃) δ 2.66 (s, 6H, CH₃), 2.70 (s, 6H, CH₃), 5.37 (d, 1H, J=12.2 Hz, CH), 7.66 (d, 1H, J=12.2 Hz, CH).

### Example 2

N-(3-Nitro-phenyl)-guanidine nitrate. A mixture of 3-nitroaniline (50 mmol, 6.90 g) in EtOH (10 mL) was cooled on an ice bath. Nitric acid (69% aq. soln.; 3.6 mL) was added dropwise. To this mixture cyanamide (50% aq soln.; 5 mL) was added. The reaction mixture was stirred at r.t. for 10 min and was then refluxed under N₂ for a further 22 h. The solvent was evaporated. The dark brown solid was washed with EtOAc/EtOH and dried under high vacuum overnight to afford the title compound as a brown solid (6.90 g, 57%). ¹H-NMR (300 MHz, DMSO-d₆) δ 7.66-7.75 (m, 2H, Ph-H), 8.09-8.14 (m, 2H, Ph-H).

[4-(2,4-Dimethyl-thiazol-5-yl)-pyrimidin-2-yl]-(3-nitro-phenyl)-amine [5]. A mixture of 3-dimethylamino-1-(2,4-dimethyl-thiazol-5-yl)-propenone (1.0 mmol, 0.21 g) and N-(3-nitro-phenyl)-guanidine nitrate (1.0 numol, 0.24 g) in 2-methoxyethanol (5 mL) was treated with NaOH (40 mg). The reaction mixture was refluxed under N₂ for 20 h. The solvent was evaporated and the residue was purified by flash chromatography (EtOAc/PE, 5:1) and recrystallisation from EtOAc/MeOH to afford the title compound as a yellow solid (151 mg, 46%). M.p. 176-178° C. LC-MS: m/z=328 (M+1). C₁₅H₁₃N₅O₂S requires: C, 55.03; H, 4.00; N, 21.39; found: C, 54.67; H, 3.88; N, 21.77. ¹H-NMR (300 MHz, CDCl₃) δ 2.72 (s, 3H, CH₃), 2.74 (s, 3H, CH₃), 7.06 (d, 1H, J=5.3 Hz, pyrimidinyl-H), 7.74-7.92 (m, 3H, Ph-H), 8.46 (d, 1H, J=5.3 Hz, pyrimidinyl-H), 8.91 (t, 1H, J=4.3, 2.1 Hz, Ph-H).

### Example 3

N-(4-Fluoro-phenyl)-guanidine nitrate. A solution of 4-fluoroaniline (25 mmol, 2.80 g) in EtOH (10 mL) was cooled on an ice bath. Nitric acid (69% aq. soln.; 1.8 mL) was added dropwise. Then cyanamide (50% aq. soln.; 4 mL) was added. The reaction mixture was refluxed under N₂ for 21 h. The solvent was evaporated to dryness. The solid residue was washed with EtOH and dried under high vacuum overnight to afford the title compound as a purple powder (2.54 g, 47%). This material was used for subsequent reaction without further purification.

[4-(2,4-Dimethyl-thiazol-5-yl)-pyrimidin-2-yl]-(4-fluoro-phenyl)-amine [8]. To a mixture of 3-dimethylamino-1-(2,4-dimethyl-thiazol-5-yl)-propenone (1.0 mmol, 0.21 g) and N-(4-fluoro-phenyl)-guanidine nitrate (2.0 mmol, 0.44 g) in 2-methoxyethanol (5 mL) was added NaOH (40 mg). The reaction mixture was refluxed under N₂ for 24 h. The solvent was evaporated to dryness and the residue was purified by flash chromatography (EtOAc/PE, 2:1) and recrystallisation from EtOAc/PE to afford the title compound as brown crystals (269 mg, 89%). ¹H-NMR (300 MHz, CDCl₃) δ 2.69 (s, 3H, CH₃), 2.71 (s, 3H, CH₃), 6.93 (d, 1H, J=5.3 Hz, pyrimidinyl-H), 7.03 (m, 2H, Ph-H), 7.58 (m, 2H, Ph-H), 8.40 (d, 1H, J=5.3 Hz, pyrimidinyl-H).

### Example 4

N-(2,4-Difluoro-phenyl)-guanidine nitrate. To a mixture of 2,4-difluoroaniline (25 mmol, 3.2 g) in EtOH (10 mL) in an ice bath was added nitric acid (69% aq soln.; 1.8 mL) dropwise. After completion of the addition cyanamide (50% aq. soln.; 4 mL) was added. The reaction mixture was refluxed under N₂ for 22 h. The solvent was evaporated. The solid residue was washed with EtOH and was dried under high vacuum to afford the title compound as a purple solid (2.32 g, 40%).

(2,4-Difluoro-phenyl)-[4-(2,4-dimethyl-thiazol-5-yl)-pyrimidin-2-yl]-amine [9]. A mixture of 3-dimethylamino-1-(2,4-dimethyl-thiazol-5-yl)-propenone (1.0 mmol, 0.21 g) and N-(2,4-difluoro-phenyl)-guanidine nitrate (2 mmol, 0.47 g) in 2-methoxyethanol (5 mL) was treated with NaOH (40 mg). After 24 h refluxing under N₂ the solvent was evaporated to dryness and the residue was purified by flash chromatography (EtOAc/PE, 2:1) and recrystallisation from EtOAc/PE to afford the title compound as a brown powder (250 mg, 79%). ¹H-NMR (300 MHz, CDCl₃) δ 2.69 (s, 3H, CH₃), 2.71 (s, 3H, CH₃), 6.93 (d, 1H, J=5.3 Hz, pyrimidinyl-H), 7.01 (m, 2H, Ph-H), 7.58 (m, 2H, Ph-H), 8.40 (d, 1H, J=5.3 Hz, pyrimidinyl-H).

### Example 5

N-(4-Hydroxy-2-nitro-phenyl)-guanidine nitrate. A mixture of 4-amino-2-nitrophenol (25 mmol, 3.85 g) in EtOH (6 mL) on an ice bath was treated with nitric acid (69% aq soln.; 1.8 mL). To this of cyanamide (50% aq. soln.; 4 mL) was added. The reaction mixture was refluxed under N₂ for 22 h. The solvent was evaporated. The dark brown solid residue was washed with EtOH and was dried under high vacuum to afford the title compound as a grey solid (3.53 g, 54%).

4-[4-(2,4-Dimethyl-thiazol-5-yl)-pyrimidin-2-ylamino]-2-nitro-phenol [32]. 3-Dimethylamino-1-(2,4-dimethyl-thiazol-5-yl)-propenone (1 mmol, 0.21 g) in 2-methoxyethanol (5 mL ) was treated with N-(4-hydroxy-2-nitro-phenyl)-guanidine nitrate (2 mmol, 0.52 g) in the presence of NaOH (40 mg). The reaction mixture was refluxed under N₂ for 24 h. The solvent was evaporated to dryness and the residue was purified by flash chromatography (EtOAc) and recrystallisation from EtOAc/PE to afford the title compound as a yellow powder (61 mg). ¹H-NMR (300 MHz, CDCl₃) δ 2.71 (s, 3H, CH₃), 2.73 (s, 3H, CH₃), 7.01 (d, 1H, J=5.2 Hz, pyrimidinyl-H), 7.18 (m, 1H, Ph-H), 7.64 (m, 1H, Ph-H), 8.42 (d, 1H, J=5.2 Hz, pyrimidinyl-H), 8.75 (d, 1H, J=2.7 Hz, Ph-H), 10.45 (br. s, 1H, OH).

The following compounds were prepared in a manner analogous to that described above:

(2-Chloro-phenyl)-[4-(2,4-dimethyl-thiazol-5-yl)-pyrimidin-2-yl]-amine [1]. ¹H-NMR (300 MHz, CDCl₃) δ 2.71 (s, 3H, CH₃), 2.72 (s, 3H, CH₃), 6.96-7.02 (m, 2H, pyrimidinyl-H and Ph-H), 7.30-7.42 (m, 2H, Ph-H), 8.46 (d, 1H, J=5.3 Hz, pyrimidinyl-H). 8.54-8.58 (m, 1H, Ph-H).

(4-Chloro-phenyl)-[4-(2,4-dimethyl-thiazol-5-yl)-pyrimidin-2-yl]-amine [2]. ¹H-NMR (300 MHz, CDCl₃) δ 2.70 (s, 3H, CH₃), 2.71 (s, 3H, CH₃), 6.96 (d, 2H, J=5.3 Hz, pyrimidinyl-H), 7.33 (m, 2H, Ph-H), 7.60 (m, 2H, Ph-H), 8.42 (d, 1H, J=5.3 Hz, pyrimidinyl-H).

(3-Chloro-phenyl)-[4-(2,4-dimethyl-thiazol-5-yl)-pyrimidin-2-yl]-amine [3]. ¹H-NMR (300 MHz, CDCl₃) δ 2.71 (s, 6H, CH₃), 6.97-7.04 (m, 2H, pyrimidinyl-H and Ph-H), 7.23-7.36 (m, 2H, Ph-H), 7.94 (t, 1H, J=1.9, 3.9 Hz, Ph-H), 8.43 (d, 1H, J=5.3 Hz, pyrimidinyl-H).

[4-(2,4-Dimethyl-thiazol-5-yl)-pyrimidin-2-yl]-(2-fluoro-phenyl)-amine [7]. ¹H-NMR (300 MHz, CDCl₃) δ 2.71 (s, 3H, CH₃), 2.72 (s, 3H, CH₃), 6.98-7.22 (m, 4H, pyrimidinyl-H and Ph-H), 8.45 (d, 1H, J=5.3 Hz, pyrimidinyl-H). 8.50 (m, 1H, Ph-H).

[4-(2,4-Dimethyl-thiazol-5-yl)-pyrimidin-2-yl]-(3-fluoro-phenyl)-amine [9]. ¹H-NMR (300 MHz, CDCl₃) δ 2.71 (s, 3H, CH₃), 2.72 (s, 3H, CH₃), 6.75 (m, 1H, Ph-H), 7.00 (d, 1H, J=5.3 Hz, pyrimidinyl-H), 7.17-7.32 (m, 3H, Ph-H), 7.77 (m, 1H, Ph-H), 8.44 (d, 1H, J=5.3 Hz, pyrimidinyl-H).

(3,5-Difluoro-phenyl)-[4-(2,4-dimethyl-thiazol-5-yl)-pyrimidin-2-yl]-amine [10]. ¹H-NMR (300 MHz, CDCl₃) δ 2.71 (s, 3H, CH₃), 2.73 (s, 3H, CH₃), 6.49 (m, 1H, Ph-H), 7.02 (d, 1H, J=5.3 Hz, pyrimidinyl-H), 7.28-7.34 (m, 2H, Ph-H), 8.46 (d, 1H, J=5.3 Hz, pyrimidinyl-H).
(3,5-Dichloro-phenyl)-[4-(2,4-dimethyl-thiazol-5-yl)-pyrimidin-2-yl]-amine [11]. ¹H-NMR (300 MHz, CDCl₃) δ 2.72 (s, 6H, CH₃), 7.01-7.04 (m, 2H, pyrimidinyl-H and Ph-H), 7.67 (m, 2H, Ph-H), 8.45 (d, 1H, J=5.3 Hz, pyrimidinyl-H).

(2,4-Dichloro-phenyl)-[4-(2,4-dimethyl-thiazol-5-yl)-pyrimidin-2-yl]-amine [12]. ¹H-NMR (300 MHz, CDCl₃) δ 2.71 (s, 3H, CH₃), 2.72 (s, 3H, CH₃), 7.02 (d, 1H, J=5.3 Hz, pyrimidinyl-H), 7.29-7.42 (m, 2H, Ph-H), 8.46 (d, 1H, J=5.3 Hz, pyrimidinyl-H), 8.54 (d, 1H, J=8.9 Hz, Ph-H).

[4-(2,4-Dimethyl-thiazol-5-yl)-pyrimidin-2-yl]-(3-trifluoromethyl-phenyl)-amine [13]. ¹H-NMR (300 MHz, CDCl₃) δ 2.71 (s, 3H, CH₃), 2.72 (s, 3H, CH₃), 7.01 (d, 1H, J=5.3 Hz, pyrimidinyl-H), 7.29-7.34 (m, 2H, Ph-H), 7.45 (m, 1H, Ph-H), 7.64 (m, 1H, Ph-H), 8.45 (d, 1H, J=5.3 Hz, pyrimidinyl-H).

[4-(2, 4-Dimethyl-thiazol-5-yl)-pyrimidin-2-yl]-(2-trifluoromethyl-phenyl)-amine [14]. ¹H-NMR (300 MHz, CDCl₃) δ 2.69 (s, 3H, CH₃), 2.70 (s, 3H, CH₃), 7.00 (d, 1H, J=5.3 Hz, pyrimidinyl-H), 7.19 (m, 1H, Ph-H), 7.59-7.65 (m, 2H, Ph-H), 8.37 (d, 1H, J=6.4 Hz, Ph-H), 8.44 (d, 1H, J=5.3 Hz, pyrimidinyl-H).

[4-(2, 4-Dim ethyl-thiazol-5-yl)-pyrimidin-2-yl]-(4-trifluoromethyl-phenyl)-amine [15]. Orange solid. M.p. 183-185° C. LC-MS: m/z=351.4 (M+1). C₁₆H₁₃F₃N₄S requires: C, 54.85; H, 3.74; N, 15.99; found: C, 54.71; H, 3.59; N, 16.26. ¹H-NMR (300 MHz, CDCl₃) δ 2.72 (s, 3H, CH₃), 2.73 (s, 3H, CH₃), 7.03 (d, 1H, J=5.3 Hz, pyrimidinyl-H), 7.60 (m, 2H, Ph-H), 7.79 (m, 2H, Ph-H), 8.46 (d, 1H, J=5.3 Hz, pyrimidinyl-H).

(2-Bromo-phenyl)-[4-(2,4-dimethyl-thiazol-5-yl)-pyrimidin-2-yl]-amine [16]. ¹H-NMR (300 MHz, CDCl₃) δ 2.71 (s, 3H, CH₃), 2.72 (s, 3H, CH₃), 6.92 (m, 1H, Ph-H), 7.00 (d, 1H, J=5.3 Hz, pyrimidinyl-H), 7.38 (m, 1H, Ph-H), 7.59 (m, 2H, Ph-H), 8.46 (d, 1H, J=5.3 Hz, pyrimidinyl-H), 8.51 (m, 1H, Ph-H).
(3-Bromo-phenyl)-[4-(2,4-dimethyl-thiazol-5-yl)-pyrimidin-2-yl]-amine [17]. ¹H-NMR (300 MHz, CDCl₃) δ 2.72 (s, 6H, CH₃), 6.98 (d, 1H, J=5.3 Hz, pyrimidinyl-H), 7.19 (m, 2H, Ph-H), 7.41 (m, 1H, Ph-H), 8.11 (m, 1H, Ph-H), 8.44 (d, 1H, J=5.3 Hz, pyrimidinyl-H).
(4-Bromo-phenyl)-[4-(2,4-dimethyl-thiazol-5-yl)-pyrimidin-2-yl]-amine [18]. Yellow solid. M.p. 173-175° C. LC-MS: m/z=363 (M+1). C₁₅H₁₃BrN₄S requires: C, 49.87; H, 3.63; N, 15.51; found: C, 49.81; H, 3.61; N, 15.56. ¹H-NMR (300 MHz, CDCl₃) δ 2.70 (s, 3H, CH3), 2.72 (s, 3H, CH3), 6.97 (d, 1H, J=5.3 Hz, pyrimidinyl-H), 7.47 (m, 2H, Ph-H), 7.55 (m, 2H, Ph-H), 8.42 (d, 1H, J=5.3 Hz, pyrimidinyl-H).
[4-(2,4-Dimethyl-thiazol-5-yl)-pyrimidin-2-yl]-(2-iodo-phenyl)-amine [19]. ¹H-NMR (300 MHz, CDCl₃) δ 2.70 (s, 3H, CH₃), 2.72 (s, 3H, CH₃), 6.80 (m, 1H, Ph-H), 6.99 (d, 1H, J=5.3 Hz, pyrimidinyl-H), 7.42 (m, 1H, Ph-H), 7.84 (m, 1H, Ph-H), 8.39 (m, 1H, Ph-H), 8.45 (d, 1H, J=5.3 Hz, pyrimidinyl-H).
[4-(2,4-Dimethyl-thiazol-5-yl)-pyrimidin-2-yl]-(3-iodo-phenyl)-amine [20]. ¹H-NMR (300 MHz, d₆-DMSO) δ: 2.68 (s, 6H, CH₃), 7.03 (m, 2H, pyrimidinyl-H and Ph-H), 7.28 (d, 1H, J=7.9 Hz, Ph-H), 7.68 (m, 1H, Ph-H), 8.41 (m, 1H, Ph-H), 8.47 (d, 1H, J=5.3 Hz, pyrimidinyl-H).

[4-(2,4-Dimethyl-thiazol-5-yl)-pyrimidin-2-yl]-(4-iodo-phenyl)-amine [21]. Yellow solid. M.p. 171-173° C. LC-MS: m/z=409 (M+1). C₁₅H₁₃IN₄S requires: C, 44.13; H, 3.21; N, 13.72; found: C, 44.03; H, 3.17; N, 13.73. ¹H-NMR (300 MHz, d₆-DMSO) δ 2.70 (s, 3H, CH₃), 2.72 (s, 3H, CH₃), 6.97 (d, 1H, J=5.3 Hz, pyrimidinyl-H), 7.46 (m, 1H, Ph-H), 7.64 (m, 2H, Ph-H), 8.42 (d, 1H, J=5.3 Hz, pyrimidinyl-H).

(3,4-Difluoro-phenyl)-[4-(2,4-dimethyl-thiazol-5-yl)-pyrimidin-2-yl]-amine [23]. ¹H-NMR (300 MHz, d₆-DMSO) δ 2.70 (s, 3H, CH₃), 2.72 (s, 3H, CH₃), 6.98 (d, 1H, J=5.3 Hz, pyrimidinyl-H), 7.11 (m, 2H, Ph-H), 7.83 (m, 1H, Ph-H), 8.43 (d, 1H, J=5.3 Hz, pyrimidinyl-H).

[4-(2, 4-Dimethyl-thiazol-5-yl)-pyrimidin-2-yl]-(2-methoxy-phenyl)-amine [24]. ¹H-NMR (300 MHz, CDCl₃) δ 2.71 (s, 3H, CH₃), 2.72 (s, 3H, CH₃), 3.92 (s, 3H, OCH₃), 6.89-7.04 (d, 4H, Ph-H and pyrimidinyl-H), 8.43 (d, 1H, J=5.3 Hz, pyrimidinyl-H), 8.53 (m, 1H, Ph-H).

[4-(2, 4-Dimethyl-thiazol-5-yl)-pyrimidin-2-yl]-(3-methoxy-phenyl)-amine [25]. ¹H-NMR (300 MHz, CDCl₃) δ 2.70 (s, 3H, CH₃), 2.71 (s, 3H, CH₃), 3.86 (s, 3H, OCH₃), 6.61 (m, 1H, Ph-H), 6.94 (d, 1H, J=5.3 Hz, pyrimidinyl-H), 7.10-7.28 (m, 3H, Ph-H), 8.42 (d, 1H, J=5.3 Hz, pyrimidinyl-H).
[4-(2,4-Dimethyl-thiazol-5-yl)-pyrimidin-2-yl]-(4-methoxy-phenyl)-amine [26]. Orange-yellow solid. M.p. 137-139° C. LC-MS: m/z=313 (M+1). C₁₆H₁₆N₄OS requires: C, 61.51; H, 5.16; N, 17.94; found: C, 61.32; H, 5.18; N, 18.36. ¹H-NMR (300 MHz, CDCl₃) δ 2.68 (s, 3H, CH₃), 2.70 (s, 3H, CH₃), 3.82 (s, 3H, OCH₃), 6.88-6.93 (d, 4H, Ph-H and pyrimidinyl-H), 7.52 (m, 1H, Ph-H), 8.37 (d, 1H, J=5.3 Hz, pyrimidinyl-H).
3-[4-(2,4-Dimethyl-thiazol-5-yl)-pyrimidin-2-ylamino]-phenol [27]. ¹H-NMR (300 MHz, d₆-DMSO) δ 2.67 (s, 3H, CH₃), 2.68 (s, 3H, CH₃), 6.42 (d, 1H, J=8.0 Hz, Ph-H), 6.94 (d, 1H, J=5.2 Hz, pyrimidinyl-H), 7.05 (m, 1H, Ph-H), 7.24 (m, 2H, Ph-H), 7.99 (m, 1H, Ph-H), 8.43 (d, 1H, J=5.2 Hz, pyrimidinyl-H), 8.99 (br. s, 1H, NH), 9.21 (br. s, 1H, OH).
4-[4-(2,4-Dimethyl-thiazol-5-yl)-pyrimidin-2-ylamino]-phenol [28]. ¹H-NMR (300 MHz, d₆-DMSO) δ 2.61 (s, 3H, CH₃), 2.64 (s, 3H, CH₃), 6.71 (m, 2H, Ph-H), 6.97 (d, 1H, J=5.2 Hz, pyrimidinyl-H), 7.49 (m, 2H, Ph-H), 7.24 (m, 2H, Ph-H), 8.43 (d, 1H, J=5.2 Hz, pyrimidinyl-H), 9.06 (br. s, 1H, NH), 9.32 (br. s, 1H, OH).

4-[4-(2,4-Dimethyl-thiazol-5-yl)-pyrimidin-2-ylamino]-benzonitrile [35]. ¹H-NMR (300 MHz, d₆-DMSO) δ 2.65 (s, 3H, CH₃), 2.67 (s, 3H, CH₃), 7.22 (d, 1H, J=5.2 Hz, pyrimidinyl-H), 7.77 (m, 2H, Ph-H), 7.99 (m, 2H, Ph-H), 8.61 (d, 1H, J=5.2 Hz, pyrimidinyl-H), 10.2 (s, 1H, NH).

3-[4-(2, 4-Dimethyl-thiazol-5-yl)-pyrimidin-2-ylamino]-benzonitrile [36]. ¹H-NMR (300 MHz, d₆-DMSO) δ 2.71 (s, 3H, CH₃), 2.72 (s, 3H, CH₃), 7.03 (d, 1H, J=5.2 Hz, pyrimidinyl-H), 7.31-7.45 (m, 2H, Ph-H), 7.67 (m, 1H, Ph-H), 8.29 (m, 1H, Ph-H), 8.45 (d, 1H, J=5.2 Hz, pyrimidinyl-H).

4-[4-(2,4-Dimethyl-thiazol-5-yl)-pyrimidin-2-ylamino]-benzoic acid methyl ester [37]. ¹H-NMR (300 MHz, CDCl₃) δ 2.72 (s, 3H, CH₃), 2.73 (s, 3H, CH₃), 3.91 (s, 3H, OCH₃), 7.02 (d, 1H, J=5.3 Hz, pyrimidinyl-H), 7.41 (sbr, 1H, NH), 7.76 (m, 2H, Ph-H), 8.05 (m, 2H, Ph-H), 8.47 (d, 1H, J=5.3 Hz, pyrimidinyl-H).

(3-Chloro-4-methyl-phenyl)-[4-(2,4-dimethyl-thiazol-5-yl)-pyrimidin-2-yl]-amine [38]. ¹H-NMR (300 MHz, CDCl₃) δ 2.45 (s, 3H, CH₃), 2.71 (s, 6H, CH₃), 6.99 (d, 1H, J=5.3 Hz, pyrimidinyl-H), 7.18-7.32 (m, 2H, Ph-H), 7.82 (m, 1H, Ph-H), 8.41 (d, 1H, J=5.3 Hz, pyrimidinyl-H).

(3-Chloro-4-methoxy-phenyl)-[4-(2,4-dimethyl-thiazol-5-yl)-pyrimidin-2-yl]-amine [39]. ¹H-NMR (300 MHz, CDCl₃) δ 2.70 (s, 3H, CH₃), 2.71 (s, 3H, CH₃), 3.90 (s, 3H, OCH₃), 6.92 (m, 2H, pyrimidinyl-H & Ph-H), 7.10 (sbr, 1H, NH), 7.38 (m, 1H, Ph-H), 7.85 (m, 1H, Ph-H), 8.40 (d, 1H, J=5.3 Hz, pyrimidinyl-H).
4-[4-(2,4-Dimethyl-thiazol-5-yl)-pyrimidin-2-ylamino]-benzoic acid [40]. ¹H-NMR (300 MHz, CDCl₃) δ 2.65 (s, 3H, CH₃), 2.67 (s, 3H, CH₃), 7.09 (d, 1H, J=5.3 Hz, pyrimidinyl-H), 7.70 (m, 2H, Ph-H), 7.82 (m, 2H, Ph-H), 8.52 (d, 1H, J=5.3 Hz, pyrimidinyl-H).

5-[4-(2,4-Dimethyl-thiazol-5-yl)-pyrimidin-2-ylamino]-2-fluoro-benzoic acid 2-methoxy-ethyl ester [82]. ¹H-NMR (300 MHz, d₆-DMSO) δ 2.64 (s, 3H, CH₃), 2.66 (s, 3H, CH₃), 3.29 (s, 3H, OCH₃), 3.66 (m, 2H, CH₂), 4.44 (m, 2H, CH₂), 7.13 (d, 1H, J=5.3 Hz, pyrimidinyl-H), 7.32 (m, 1H, Ph-H), 7.98 (m, 1H, Ph-H), 8.39 (m, 1H, Ph-H), 8.54 (d, 1H, J=5.3 Hz, pyrimidinyl-H), 9.93 (s, 1H, NH).

### Example 6

4-(2,4-Dimethyl-thiazol-5-yl)-pyrimidin-2-ylamine. This compound was prepared by heating equimolar amounts of 3-dimethylamino-1-(2,4-dimethyl-thiazol-5-yl)-propenone and guanidine in refluxing 2-methoxethanol. ¹H-NMR (300 MHz, CDCl₃) δ 2.67 (s, 3H, CH₃), 2.68 (s, 3H, CH₃), 5.14 (br, 2H, NH₂), 6.83 (d, 1H, J=5.3 Hz, pyrimidinyl-H), 8.30 (d, 1H, J=5.3 Hz, pyrimidinyl-H).

N-[4-(2,4-Dimethyl-thiazol-5-yl)-pyrimidin-2-yl]-3-nitro-benzenesulfonamide [29]. A solution of 4-(2,4-dimethyl-thiazol-5-yl)-pyrimidin-2-ylamine (1 mmol, 0.227 g), 3-nitrobenenesulfonyl chloride (1.5 mmol, 0.33 g) in pyridine (4 mL) was stirred at r.t. for 24 h. The reaction mixture was evaporated to dryness. The dark brown residue was dissolved in EtOAc and was washed with 2 M aq HCl solution, water, brine and was dried over MgSO₄. Concentration gave a light yellow residue and this was purified by flash chromatography (EtOAc/PE, 5:1) and recrystallisation from EtOAc/MeOH to afford the title compound as yellow crystals (44 mg). ¹H-NMR (300 MHz, CDCl₃) δ 2.68 (s, 3H, CH₃), 2.73 (s, 3H, CH₃), 7.59 (d, 1H, J=5.3 Hz, pyrimidinyl-H), 7.90 (m, 1H, Ph-H), 8.60 (m, 1H, Ph-H), 8.75 (m, 1H, Ph-H), 8.81 (d, 1H, J=5.4 Hz, pyrimidinyl-H), 9.15 (t, 1H, J=1.98, 3.91 Hz, Ph-H).

### Example 7

3-Dimethylamino-1-(4-methyl-2-methylamino-thiazol-5-yl)-propenone. A solution of 3-chloro-2,4-pentadione (2.5 g, 19 mmol) in MeOH (15 mL) treated with N-methyl-2-thiourea (1.67 g, 19 mmol) and pyridine (1.5 mL). The reaction mixture was stirred at r.t. for 2-3 h. The resulting precipitates were filtered and washed with Et₂O to afford a white solid product of 5-acetyl-2-methylamino-4-methylthiazol, which was used in the next reaction step without further purification. A mixture of this product (2.05 g) in N,N-dimethylformamide dimethyl acetal (10 mL) was heated at 100-110° C. for 22 h. The reaction mixture was concentrated. The precipitate was collected and washed with EtOAc to afford the title compound as an orange solid. ¹H-NMR (300 MHz, CDCl₃) δ 2.55, 2.94 (s, 6H, CH₃), 3.40 (s, 6H, NCH₃), 5.29 (d, 1H, J=12.2 Hz, CH), 7.62 (d, 1H, J=12.2 Hz, CH).

### Example 8

(4-Fluoro-phenyl)-[4-(4-methyl-2-methylamino-thiazol-5-yl)-pyrimidin-2-yl]-amine [47]. A mixture 3-dimethylamino-1-(4-methyl-2-methylamino-thiazol-5-yl)-propenone (1 mmol, 0.22 g) and N-(4-fluoro-phenyl)-guanidine nitrate (2 mmol, 0.44 g) in 2-methoxyethanol (5 mL) was added NaOH (40 mg). The reaction mixture was heated at 110-120° C. under N₂ for 20 h. The solvent was evaporated to dryness and the residue was purified by flash chromatography, using EtOAc/PE (1:1, v/v) to elute the product as a yellow solid. Recrystallisation from EtOAc/MeOH yielded 230 mg brown crystals of pure title compound. ¹H-NMR (300 MHz, d₆-DMSO) δ 2.46 (s, 3H, CH₃), 2.86 (d, 3H, CH₃), 6.90 (d, 1H, J=5.4 Hz, pyrimidinyl-H), 7.11 (m, 2H, Ph-H), 7.76 (m, 2H, Ph-H), 8.07 (m, 1H, NH), 8.32 (d, 1H, J=5.4 Hz, pyrimidinyl-H), 9.48 (s, 1H, NH).

The following compounds were prepared in a manner analogous to that described above:

4-[4-(4-Methyl-2-methylamino-thiazol-5-yl)-pyrimidin-2-ylamino]-phenol [48]. ¹H-NMR (300 MHz, CD₃OD₃) δ 2.53 (s, 3H, CH₃), 2.98 (s, 3H, CH₃), 6.77 (d, 2H, J=8.8 Hz, Ph-H), 6.86 (d, 1H, J=5.5Hz, pyrimidinyl-H), 7.44 (d, 2H, J=8.8 Hz, Ph-H), 8.21 (d, 1H, J=5.5 Hz, pyrimidinyl-H).

(4-Iodo-phenyl)-[4-(4-methyl-2-methylamino-thiazol-5-yl)-pyrimidin-2-yl]-amine [57]. Yellow solid. ¹H-NMR (300 MHz, d₆-DMSO) δ 2.50 (s, 3H, CH₃), 2.92 (d, 6H, CH₃), 6.85 (d, 1H, J=5.4 Hz, pyrimidinyl-H), 7.53 (d, 2H, J=8.8 Hz, Ar-H), 7.65 (d, 2H, J=8.8 Hz, Ar-H), 8.28 (d, 1H, J=5.4 Hz, pyrimidinyl-H) 9.41 (s, 1H, NH).

[4-(4-Methyl-2-methylamino-thiazol-5-yl)-pyrimidin-2-yl]-(3-nitro-phenyl)-amine [61]. Yellow solid. ¹H-NMR (300 MHz, d₆-DMSO) δ2.80 s, 3H, CH₃), 3.09 (s, 3H, CH₃), 7.01 (d, 1H, J=5.4 Hz, pyrimidinyl-H), 7.55 (m, 1H, Ph-H), 7.79 (d, 1H, Ph-H), 8.02 (d, 1H, Ph-H), 8.15 (m, 1H, NH), 8.41 (d, 1H, J=5.4 Hz, pyrimidinyl-H), 9.00 (s, 1H, Ph-H), 10.02 (s, 1H, NH). DE MALDI-TOF MS: [M+H]⁺=345.15 (C₁₅H₁₄N₆O₂S requires 342.38).

(3-Bromo-phenyl)-[4-(4-methyl-2-methylamino-thiazol-5-yl)-pyrimidin-2-yl]-amine [68]. Yellow solid. ¹H-NMR (300 MHz, d₆-DMSO) δ 2.87 (s, 3H, CH₃), 3.11 (s, 3H, CH₃), 6.96 (d, 1H, J=5.4 Hz, pyrimidinyl-H), 7.10 (m, 1H, Ph-H), 7.23 (m, 1H, Ph-H), 7.62 (m, 1H, Ph-H), 8.15 (m, 1H, NH), 8.31 (s, 1H, Ph-H), 8.38 (d, 1H, J=5.0 Hz, pyrimidinyl-H), 9.70 (s, 1H, NH). DE MALDI-TOF MS: [M+H]⁺=377.4 (C₁₅H₁₄N₆SBr requires 376.3).

3-[4-(4-Methyl-2-methylamino-thiazol-5-yl)-pyrimidin-2-ylamino]-phenol [70]. Yellow crystals. ¹H-NMR (300 MHz, d₆-DMSO) δ2.86 (s, 3H, CH₃), 3.24 (s, 3H, CH₃), 6.36 (m, 1H, Ph-H), 6.88 (d, 1H, J=5.5 Hz, pyrimidinyl-H), 7.03 (m, 1H, Ph-H), 7.24 (m, 1H, Ph-H), 8.06 (m, 1H, NH), 8.32 (d, 1H, J=4.5 Hz, pyrimidinyl-H), 9.21 (s, 1H, Ph-H), 9.31 (s, 1H, NH). DE MALDI-TOF MS: [M+H]⁺=315.92 (C₁₅H₁₅N₆OS requires 313.38).

(4-Bromo-phenyl)-[4-(4-methyl-2-methylamino-thiazol-5-yl)-pyrimidin-2-yl]-amine [71]. Yellow-brown solid. ¹H-NMR (300 MHz, d₆-DMSO) δ2.86 (s, 3H, CH₃), 3.09 (s, 3H, CH₃), 6.93 (d, 1H, J=5.5 Hz, pyrimidinyl-H), 7.43 (m, 2H, Ph-H), 7.75 (m, 2H, Ph-H), 8.07 (m, 1H, NH), 8.34 (d, 1H, J=5.5 Hz, pyrimidinyl-H), 9.61 (s, 1H, NH). DE MALDI-TOF MS: [M+H]⁺=378.8 (C₁₅H₁₄N₆SBr requires 376.28).

(4-Chloro-phenyl)-[4-(4-methyl-2-methylamino-thiazol-5-yl)-pyrimidin-2-yl]-amine [72]. Tan crystals. ¹H-NMR (300 MHz, d₆-DMSO) δ 2.87 (s, 3H, CH₃), 3.23 (s, 3H, CH₃), 6.94 (d, 1H, J=5.3 Hz, pyrimidinyl-H), 7.32 (m, 2H, Ph-H), 7.81 (m, 2H, Ph-H), 8.09 (m, 1H, NH), 8.35 (d, 1H, J=5.7 Hz, pyrimidinyl-H), 9.61 (s, 1H, NH). DE MALDI-TOF MS: [M+H]⁺=332.1 (C₁₅H₁₄N₆SCl requires 331.8).

(3-Methoxy-phenyl)-[4-(4-methyl-2-methylamino-thiazol-5-yl)-pyrimidin-2-yl]-amine [73]. Light-yellow solid. ¹H-NMR (300 MHz, d₆-DMSO) δ 2.85 (s, 3H, CH₃), 3.09 (s, 3H, CH₃), 3.78 (s, 3H, CH₃), 6.52 (m, 1H, Ph-H), 6.92 (d, 1H, J=5.5 Hz, pyrimidinyl-H), 7.16 (m, 1H, Ph-H), 7.29 (m, 1H, Ph-H), 7.56 (s, 1H, Ph-H), 8.10 (m, 1H, NH), 8.35 (d, 1H, J=5.3 Hz, pyrimidinyl-H), 9.45 (s, 1H, NH). DE MALDI-TOF MS: [M+H]⁺=327.8 (C₁₆H₁₇N₅OS requires 327.4).

[4-(4-Methyl-2-methylamino-thiazol-5-yl)-pyrimidin-2-yl]-(4-trifluoromethyl-phenyl)-amine [74]. Yellow-brown solid. ¹H-NMR (300 MHz, d₆-DMSO) δ 2.88 (s, 3H, CH₃), 3.10 (s, 3H, CH₃), 7.01 (d, 1H, J=5.5 Hz, pyrimidinyl-H), 7.62 (m, 2H, Ph-H), 8.01 (m, 2H, Ph-H), 8.12 (m, 1H, NH), 8.40 (d, 1H, J=5.5 Hz, pyrimidinyl-H), 9.91 (s, 1H, NH). DE MALDI-TOF MS: [M+H]⁺=365.5 (C₁₆H₁₄N₅SF₃ requires 365.4).

[4-(4-Methyl-2-methylamino-thiazol-5-yl)-pyrimidin-2-yl]-(3-trifluoromethyl-phenyl)-amine [75]. Yellow-brown solid. ¹H-NMR (300 MHz, d₆-DMSO) δ 2.86 (s, 3H, CH₃), 3.11 (s, 3H, CH₃), 6.99 (d, 1H, J=5.5 Hz, Ph-H), 7.27 (m, 1H, Ph-H), 7.50 (m, 1H, Ph-H), 7.87 (m, 1H, Ph-H), 8.15 (m, 1H, NH), 8.40 (d, 1H, J=5.4 Hz, pyrimidinyl-H), 8.47 (s, 1H, Ph-H), 9.86 (s, 1H, NH). DE MALDI-TOF MS: [M+H]⁺=369.8 (C₁₆H₁₄N₅SF₃ requires 365.4).

2-Chloro-4-[4-(2,4-dimethyl-thiazol-5-yl)-pyrimidin-2-ylamino]-benzoic acid methyl ester [85]. Yellow crystals. ¹H-NMR (30 MHz, d₆-DMSO) δ 2.88 (s, 3H, CH₃), 3.10 (s, 3H, CH₃), 3.82 (s, 3H, CH₃), 7.05 (d, 1H, J=5.5, pyrimidinyl-H), 7.73 (d, 1H, J=8.8 Hz, Ph-H), 7.85 (d, 1H, J=8.7 Hz, Ph-H), 8.20 (m, 1H, NHCH₃), 8.27 (s, 1H, Ph-H), 8.43 (d, 1H, J=5.6Hz, pyrimidinyl-H). DE MALDI-TOF MS: [M+H]⁺=388.8 (C₁₇H₁₆N₅O₂SCl requires 389.9).

(3-Iodo-phenyl)-[4-(4-methyl-2-methylamino-thiazol-5-yl)-pyrimidin-2-yl]-amine [86] Yellow crystals. ¹H-NMR (30 MHz, d₆-DMSO) δ 2.88 (s, 3H, CH₃), 3.10 (s, 3H, CH₃), 6.96 (d, 1H, J=5.3 Hz, pyrimidinyl-H), 7.07 (m, 1H, Ph-H), 7.28 (m, 1H, Ph-H), 7.61 (m, 1H, Ph-H), 8.14 (m, 1H, NH), 8.37 (d, 1H, J=5.3 Hz, pyrimidinyl-H), 8.50 (s, 1H, Ph-H), 9.64 (s, 1H, NH). DE MALDI-TOF MS: [M+H]⁺=423.3 (C₁₅H₁₄N₆SI requires 423.3).

(3-Fluoro-phenyl)-[4-(4-methyl-2-methylamino-thiazol-5-yl)-pyrimidin-2-yl]-amine [87]. Yellow solid. ¹H-NMR (300 MHz, d₆-DMSO) δ 2.87 (s, 3H, CH₃), 3.10 (s, 3H, CH₃), 6.74 (m, 1H, Ph-H), 6.97 (d, 1H, J=5.4Hz, pyrimidinyl-H), 7.29 (m, 1H, Ph-H), 7.47 (m, 1H, Ph-H), 7.87 (m, 1H, Ph-H), 8.12 (m, 1H, NH), 8.38 (d, 1H, J=5.3 Hz, pyrimidinyl-H), 9.71 (s, 1H, NH). DE MALDI-TOF MS: [M+H]⁺=316.3 (C₁₅H₁₄N₅SF requires 315.4).

(3,4-Difluoro-phenyl)-[4-(4-methyl-2-methylamino-thiazol-5-yl)-pyrimidin-2-yl]-amine [88]. Light-yellow solid. ¹H-NMR (300 MHz, d₆-DMSO) δ 2.87 s, 3H, CH₃), 3.12 (s, 3H, CH₃), 6.97 (d, 1H, J=5.1 Hz, pyrimidinyl-H), 7.35 (m, 1H, Ph-H), 8.04 (d, 1H, Ph-H), 8.08 (d, 1H, Ph-H), 8.20 (m, 1H, NH), 8.37 (d, 1H, J=5.3, pyrimidinyl-H), 9.71 (s, 1H, NH). DE MALDI-TOF MS: [M+H]⁺=333.8 (C₁₅H₁₃N₅SF₂ requires 333.4).

(2,4-Difluoro-phenyl)-[4-(4-methyl-2-methylamino-thiazol-5-yl)-pyrimidin-2-yl]-amine [89]. Light-yellow solid. ¹H-NMR (300 MHz, d₆-DMSO) δ: 2.84 (s, 3H, CH₃), 3.10 (s, 3H, CH₃), 6.86 (d, 1H, J=5.5 Hz, pyrimidinyl-H), 7.06 (m, 1H, Ph-H), 7.29 (m, 1H, Ph-H), 7.67 (m, 1H, Ph-H), 8.04 (m, 1H, NH), 8.26 (d, 1H, J=5.3, pyrimidinyl-H), 8.92 (s, 1H, NH). DE MALDI-TOF MS: [M+H]⁺=334.2 (C₁₅H₁₃N₅SF₂ requires 333.4).

(3,5-Difluoro-phenyl)-[4-(4-methyl-2-methylamino-thiazol-5-yl)-pyrimidin-2-yl]-amine [90]. Yellow solid. ¹H-NMR (300 MHz, d₆-DMSO) δ 2.87 (s, 3H, CH₃), 3.10 (s, 3H, CH₃), 6.74 (m, 1H, Ph-H), 7.02 (d, 1H, J=5.5, pyrimidinyl-H), 7.60 (m, 2H, Ph-H), 8.18 (m, 1H, NH), 8.41 (d, 1H, J=5.4 Hz, pyrimidinyl-H), 9.92 (s, 1H, NH). DE MALDI-TOF MS: [M+H]⁺=333.4 (C₁₅H₁₃N₅SF₂ requires 333.4).

(4-Chloro-3-trifluoromethyl-phenyl)-[4-(4-methyl-2-methylamino-thiazol-5-yl)-pyrimidin-2-yl]-amine [91]. Light-yellow crystals. ¹H-NMR (300 MHz, d₆-DMSO) δ 2.86 (s, 3H, CH₃), 3.10 (s, 3H, CH₃), 7.01 (d, 1H, J=5.4 Hz, pyrimidinyl-H), 7.61 (m, 1H, Ph-H), 7.92 (m, 1H, Ph-H), 8.17 (m, 1H, NH), 8.40 (d, 1H, J=5.5 Hz, Ph-H), 8.53 (s, 1H, Ph-H), 9.96 (s, 1H, NH). DE MALDI-TOF MS: [M+H]⁺=399.8 (C₁₆H₁₃N₅SClF₃ requires 399.8).

(3-Chloro-phenyl)-[4-(4-methyl-2-methylamino-thiazol-5-yl)-pyrimidin-2-yl]-amine [92]. Yellow crystals. ¹H-NMR (300 MHz, d₆-DMSO) δ 2.86 (s, 3H, CH₃), 3.10 (s, 3H, CH₃), 6.95 (d, 2H, J=5.7 Hz, pyrimidinyl-H), 7.29 (m, 1H, Ph-H), 7.61 (m, 1H, Ph-H), 8.14 (s, 1H, Ph-H), 8.38 (d, 1H, J=4.3 Hz, pyrimidinyl-H), 9.72 (s, 1H, NH). DE MALDI-TOF MS: [M+H]⁺=331.6 (C₁₅H₁₄N₆SCl requires 331.8).

(4-Methoxy-phenyl)-[4-(4-methyl-2-methylamino-thiazol-5-yl)-pyrimidin-2-yl]-amine [93]. Green-yellow solid. ¹H-NMR (300 MHz, d₆-DMSO) δ 2.87 (s, 3H, CH₃), 3.35 (s, 3H, CH₃), 3,74 (s, 3H, CH₃), 6.85 (m, 1H, pyrimidinyl-H), 6.86 (m, 2H, Ph-H), 7.66 (m, 2H, Ph-H), 8.02 (m, 1H, NHCH₃), 8.29 (d, 1H, J=5.4 Hz, pyrimidinyl-H), 9.25 (s, 1H, NH). DE MALDI-TOF MS: [M+H]⁺=327.8 (C₁₆H₁₇N₅OS requires 327.4).

### Example 9

3-Dimethylamino-1-(4-methyl-2-pyridin-3-yl-thiazol-5-yl)-propenone. A mixture of 5-chloro-pentadione (5.12 g, 38 mmol) and thionicotinamide (5.25 g, 38 mmol) in MeOH (10 mL) was treated with pyridine (3 mL). The reaction mixture was heated at 70-75° C. for 5 h. The solvent was evaporated. The resulting solid was filtered and washed with EtOAc/MeOH to afford 4.33 g 5-acetyl-4-methyl-2-(3-pyridyl)-thiazol as a yellow solid, which was subjected to the next reaction without further purification. A mixture of this material (2.0 g) and NN-dimethylformamide dimethyl acetal (4 mL) was heated at 80° C. for 22 h. The reaction mixture was concentrated and then triturated with EtOAc/PE. The precipitates were collected and washed with EtOAc/PE to afford the title compound (2.05 g, 75%) as a grey solid. ¹H-NMR (300 MHz, CDCl₃) δ 2.80 (s, 6H, CH₃), 3.50 (s, 3H, CH₃), 5.47 (d, 1H, J=12.1 Hz, CH), 7.39 (m, 1H, Ar-H), 7.78 (d, 1H, J=12.1 Hz, CH), 8.28 (m, 1H, Ar-H), 8.66 (m, 1H, Ar-H), 9.16 (s, 1H, Ar-H).

### Example 10

[4-(4-Methyl-2-pyridin-3-yl-thiazol-5-yl)-pyrimidin-2-yl]-(3-nitro-phenyl)-amine [56]. To a mixture of 3-dimethylamino-1-(4-methyl-2-pyridin-3-yl-thiazol-5-yl)-propenone (1 mmol, 0.27 g) and N-(3-nitro-phenyl)-guanidine nitrate (1 mmol, 0.24 g) in 2-methoxyethanol (5 mL) was added NaOH (40 mg). The reaction mixture was heated at 120° C. under N₂ for 20 h. The solvent was evaporated to dryness and the residue was purified by flash chromatography, using EtOAc/PE (2:1, v/v) to elute the product, which was recrystallized from MeOH to afford the title compound (154 mg) as light-yellow crystals. ¹H-NMR (300 MHz, d₆-DMSO) δ 2.82 (s, 3H, CH₃), 7.24 (d, 1H, J=5.2 Hz, pyrimidinyl-H), 7.53 (m, 2H, Ar-H), 7.82 (m, 1H, Ph-H), 8.00 (m, 1H, Ar-H), 8.09 (s, 1H, Ar-H), 8.35 (m, 1H, Ar-H), 8.61 (d, 1H, J=5.2 Hz, Py-H), 8.68 (m, 1H, Ar-H), 10.23 (s, 1H, NH).

The following compound was prepared in a manner analogous to that described above:

(4-Fluoro-phenyl)-[4-(4-methyl-2-pyridin-3-yl-thiazol-5-yl)-pyrimidin-2-yl]-amine [52]. ¹H-NMR (300 MHz, d₆-DMSO) δ 2.78 (s, 3H, CH₃), 7.22 (m, 2H, pyrimidinyl-H, Ar-H), 7.59 (m, 1H, Ar-H), 7.82 (m, 2H, Ar-H), 8.38 (m, 1H, Ar-H), 8.60 (d, 1H, J=5.2 Hz, pyrimidinyl-H), 8.72 (m, 1H, Ar-H), 9.21 (s, 1H, Ar-H), 9.83 (s, 1H, NH).

### Example 11

1-(2,4-Dimethyl-thiazol-5-yl)-3-(4-trifluoromethyl-phenyl)-propenone. To an ice-cold solution of NaOH (2.2 g) in H₂O (10 mL) 2,4-dimethyl-5-acetylthiazol (43 mmol, 6.6 g) was added. After 5 min stirring this was treated with trifluoro-p-tolualdehyde (43 mmol, 7.49 g). The reaction mixture was warmed to r.t. and stirred for 2 h. It was diluted with CH₂Cl₂, washed with HCl/H₂O, brine and was dried over MgSO₄. The solvent was evaporated to afford the title compound (4.86 g).

### Example 12

4-[4-(2, 4-Dimethyl-thiazol-5-yl)-6-(4-trifluoromethyl-phenyl)-pyrimidin-2-ylamino]-2-nitro-phenol [51]. A mixture of 1-(2,4-dimethyl-thiazol-5-yl)-3-(4-trifluoromethyl-phenyl)-propenone (1 mmol, 0.31 g) and N-(4-hydroxy-3-nitro-phenyl)-guanidine nitrate (1.5 mmol, 0.39 g) in 2-methoxyethanol (5 mL) was added NaOH (40 mg). The reaction mixture was heated at 120° C. under N₂ for 20 h. The solvent was evaporated to dryness and the residue was purified by flash chromatography, using EtOAc/PE (2:1, v/v) to elute the product, which was recrystallized from MeOH/EtOAc to afford the title compound (178 mg) as orange crystals. ¹H-NMR (300 MHz, CDCl₃) δ 2.75 (s, 3H, CH₃), 2.79 (s, 3H, CH₃), 7.18 (m, 1H, Ar-H), 7.44 (s, 1H, pyrimidinyl-H), 7.61 (m, 1H, Ar-H), 7.81 (m, 2H, Ar-H), 8.22 (m, 2H, Ar-H), 8.98 (m, 1H, Ar-H).

The following compounds were prepared in a manner analogous to that described above:

[4-(2,4-Dimethyl-thiazol-[-yl)-6-phenyl-pyrimidin-2-yl]-(3-nitro-phenyl)-amine [42]. ¹H-NMR (300 MHz, d₆-DMSO) δ 2.68(s, 3H, CH₃), 2.75 (s, 3H, CH₃), 7.61 (m, 4H, Ar-H), 7.84 (m, 1H, Ar-H), 8.08 (m, 1H, Ar-H), 8.27 (m, 2H, Ar-H), 9.15 (s, 1H, Ar-H), 10.3 (s, 1H, NH).

[4-(2, 4-Dimethyl-thiazol-5-yl)-6-(4-trifluoromethyl-phenyl)-pyrimidin-2-yl]-(4-fluoro-phenyl)-amine [49]. ¹H-NMR (300 MHz, CDCl₃) δ 2.73 (s, 3H, CH₃), 2.78 (s, 3H, CH₃), 7.05 (m, 2H, Ar-H), 7.36 (s, 1H, pyrimidinyl-H), 7.78 (m, 4H, Ar-H), 8.22 (m, 2H, Ar-H), 8.67 (sbr, 1H, NH).

(4-Chloro-phenyl)-[4-(2,4-dimethyl-thiazol-5-yl)-6-(4-trifluoromethyl-phenyl)-pyrimidin-2-yl]-amine [50]. ¹H-NMR (300 MHz, CDCl₃) δ 2.73 (s, 3H, CH₃), 2.79 (s, 3H, CH₃), 7.29 (m, 2H, Ar-H), 7.39 (s, 1H, pyrimidinyl-H), 7.80 (m, 4H, Ar-H), 8.22 (m, 2H, Ar-H), 8.96 (sbr, 1H, NH).

4-[6-(2,4-Dimethyl-thiazol-5-yl)-2-(4-fluoro-phenylamino)-pyrimidin-4-yl]-phenol (55]. ¹H-NMR (300 MHz, d₆-DMSO) δ 2.67(s, 3H, CH₃), 2.72 (s, 3H, CH₃), 6.93 (m, 2H, Ar-H), 7.18 (m, 2H, Ar-H), 7.42 (s, 1H, pyrimidinyl-H), 7.84 (m, 2H, Ar-H), 8.09 (m, 2H, Ar-H), 9.67 (s, 1H, NH or OH), 10.11 (s, 1H,NH or OH).

### Example 13

[4-(2-Allylamino-4-methyl-thiazol-6-yl)-pyrimidin-2-yl]-(3-nitro-phenyl)-amine [69]. To a mixture of 1-(2-allylamino-4-methyl-thiazol-5-yl)-3-dimethylamino-propenone (1.0 mmol, 0.25 g) and N-(3-nitro-phenyl)-guanidine nitrate (1.5 mmol, 0.36 g) in 2-15 methoxyethanol (5 mL) was added NaOH (40 mg). The reaction mixture was heated at 110-120° C. under N₂ for 22 h. The solvent was evaporated to dryness and the residue was purified by flash chromatography, using EtOAc/PE (1:1, v/v) to elute the product as yellow solid. Recrystallisation from EtOAc/MeOH yielded the title compound as brown crystals. ¹H-NMR (300 MHz, d₆-DMSO) δ 2.51 (s, 3H, CH₃), 3.92 (sbr, 2H, CH₂), 5.20 (m, 2H, CH₂), 5.91 (m, 1H, CH), 7.02 (d, 1H, J=5.5 Hz, pyrimidinyl-H), 7.57 (m, 1H, Ph-H), 7.80 (m, 1H, Ph-H), 8.06 (m, 1H, Ph-H), 8.43 (d, 1H, J=5.5 Hz, pyrimidinyl-H), 8.94 (s, 1H, Ph-H), 10.04 (s, 1H, NH).

The following compound was prepared in a manner analogous to that described above:

[4-(2-Allylamino-4-methyl-thiazol-5-yl)-pyrimidin-2-yl]-(4-fluoro-phenyl)-amine [67]. ¹H-NMR (300 MHz, d₆-DMSO) δ 2.51 (s, 3H, CH₃), 3.92 (sbr, 2H, CH₂), 5.24 (m, 2H, CH₂), 5.91 (m, 1H, CH), 6.90 (d, 1H, J=5.5 Hz, pyrimidinyl-H), 7.11 (m, 2H, Ph-H), 7.76 (m, 2H, Ph-H), 8.33 (d, 1H, J=5.5 Hz, pyrimidinyl-H), 9.49 (s, 1H, NH). DE MALDI-TOF MS: [M+H]⁺=341.4 (C₁₇H₁₆FN₅S requires 341.4).

### Example 14

[4-(2-Ethylamino-4-methyl-thiazol-5-yl)-pyrimidin-2-yl]-(4-fluoro-phenyl)-amine [60]. A mixture of 3-dimethylamino-1-(2-ethylamino-4-methyl-thiazol-5-yl)-propenone (1 mmol, 0.24 g) and NaOH (40 mg) in 2-methoxylethanol (5 mL) was treated with of N-(4-fluoro-phenyl)-guanidine nitrate (0.36 g, 1.5 mmol). The reaction mixture was heated at 110-120° C. under N₂ for 20 h. After concentration, the residue was filtered and washed with MeOH. Recrystallisation from EtOAc/MeOH afforded the title compounds (291 mg) as a yellow solid. ¹H-NMR (300 MHz, d₆-DMSO) δ 1.17 (m, 3H, CH₃), 2.51 (s, 3H, CH₃), 3.26 (m, 2H, CH₂), 6.89 (d, 1H, J=5.5 Hz, pyrimidinyl-H), 7.11 (m, 2H, Ph-H), 7.77 (m, 2H, Ph-H), 8.33 (d, 1H, J=5.5 Hz, pyrimidinyl-H). DE MALDI-TOF MS: [M+H]⁺=331.2 (C₁₆H₁₆FN₅S requires 329.4).

### Example 15

4-(4-[2-(4-Nitro-phenylamino)-thiazol-5-yl]-pyrimidin-2-ylamino)-phenol [95]. A mixture of 3-dimethylamino-1-[2-(4-nitro-phenylamino)-thiazol-5-yl]-propenone (1 mmol, 0.32 g) and NaOH (50 mg) in 2-methoxylethanol (5 mL) was treated with N-(4-hydroxy-phenyl)-guanidine nitrate (0.32 g, 1.5 mmol). The reaction mixture was heated at 110-120° C. under N₂ for 6 h. After concentration, the residue was filtered and washed with MeOH. Recrystallisation from MeOH afforded the title compound as an orange solid. ¹H-NMR (300 MHz, d₆-DMSO) δ 6.67 (m, 2H, Ph-H), 6.93 (d, 1H, J=5 .4 Hz, pyrimidinyl-H), 7.48 (m, 2H, Ph-H), 7.86 (m, 2H, Ph-H), 8.26 (m, 2H, Ph-H), 8.36 (d, 1H, J=5.3 Hz, pyrimidinyl-H). DE MALDI-TOF MS: [M+H]⁺=406.82 (C₁₉H₁₄N₆O₃S requires 406.42).

### Example 16

N-{3-[4-(2,4-Dimethyl-thiazol-5-yl)-pyrimidin-2-ylamino]-phenyl}-guanidine [99]. To a mixture of [4-(2,4-dimethyl-thiazol-5-yl)-pyrimidin-2-yl]-(3-nitro-phenyl)-amine (3.97 mmol, 1.3 g) in 2-methoxyethanol (15 mL) was added AcOH (1 mL). The reaction mixture was stirred under N₂ for 10 min. Palladium catalyst (660 mg; 10% on activated carbon) was the added and the reaction mixture was allowed to stir under H₂ for 18 h. The reaction mixture was passed through Celite 521 and the precipitates were washed several times with MeOH. The filtrate was concentrated and recrystallised from MeOH/EtOAc to afford grey crystals of N-[4-(2,4-dimethyl-thiazol-5-yl)-pyrimidin-2-yl]-benzene-1,3-diamine. An aliquot of this material (500 mg) in 2-methoxylethanol was cooled on an ice bath and was treated with HCl (conc. 1 mL). Cyanamide (50% aq soln., 4 mL) was added dropwise. After completion of the addition the reaction mixture was warmed to r.t. and heated at reflux for 20 h. The reaction mixture was concentrated. The residue was diluted with EtOAc and washed with water and brine. The organic phase was evaporated and purified by chromatography, using EtOAc/MeOH (3:1, v/v) to elute the title compound. DE MALDI-TOF MS: [M+H]⁺=339.16 (C₁₆H₁₇N₇S requires 339.42).

### Example 17

{3-[4-(2,4-Dimethyl-thiazol-5-yl)-pyrimidin-2-ylamino]-phenyl}-methanol [100]. A mixture of 3-dimethylamino-1-(2,4-dimethyl-thiazol-5-yl)-propenone (10 mmol, 2.1 g) in 2-methoxylethanol was treated with N-(4-hydroxymethyl-phenyl)-guanidine hydrochloride (1.65 g) in the presence of NaOH (400 mg). The reaction mixture was allowed to reflux for 20 h. After concentration, the precipitates were filtered and washed with EtOAc/MeOH several times. Recrystallisation from MeOH/EtOAc afforded the title compound (2.17 g, 70%). ¹H-NMR (300 MHz, d₆-DMSO) δ 3.00 (s, 3H, CH₃), 3.02 (s, 3H, CH₃), 4.86 (s, 2H, CH₂), 7.30 (m, 1H, Ph-H), 7.44 (d, 1H, J=6.1 Hz, pyrimidinyl-H), 7.61 (m, 1H, Ph-H), 8.01 (m, 1H, Ph-H), 8.13 (s, 1H, Ph-H), 8.88 (d, 1H, J=6.1 Hz, pyrimidinyl-H).

### Example 18

[3-(2-Diethylamino-ethoxymethyl)-phenyl]-[4-(2,4-dimethyl-thiazol-5-yl)-pyrimidin-2-yl]-amine [102]. A solution of {3-[4-(2,4-dimethyl-thaizol-5-yl)-pyrimidin-2-ylamino]-phenyl}-methanol (1 mmol, 0.34 g) in dry DMF was treated with NaH (1 mmol, 24 mg). After stirring at r.t. for 20 min, (2-chloro-ethyl)-diethyl-amine hydrochloride (0.17 g, 1 mmol) and pyridine (0.4 mL) were added. After stirring at r.t. for 21 h the reaction mixture was cooled on an ice bath and water was added dropwise. The reaction mixture was neutralised by addition of aq HCl soln. and extracted with EtOAc. The organic phases were combined, washed with brine and dried over MgSO₄. The solvent was evaporated to dryness. The residue was purified by chromatography, using EtOAc/MeOH (1:1, v/v) to elute the title compound as light-yellow solid, which was recrystallised from EtOAc/PE. ¹H-NMR (CDCl₃) δ 1.00 (t, 6H, J=7.0 Hz, CH₃), 2.59 (m, 2H, CH₂), 2.62 (s, 3H, CH₃), 2.66 (s, 3H, CH₃), 2.78 (m, 2H, CH₂), 4.12 (m, 2H, CH₂), 4.72 (s, 2H, CH₂), 6.76 (d, 1H, J=5.5 Hz, pyrimidinyl-H), 7.24 (m, 3H, Ph-H), 7.36 (m, 1H, Ph-H), 7.40 (m, 2H, Ph-H), 8.28 (d, 1H, J=5.5 Hz, pyrimidinyl-H). DE MALDI-TOF MS: [M+H]⁺=416.15 (C₂₂H₂₉N₅SO requires 411.56).

### Example 19

[4-(2,4-Dimethyl-thiazol-5-yl)-pyrimidin-2-yl]-(4-pyridin-4-ylmethyl-phenyl)-amine [101]. A solution of 4-(4-nitro-benzyl)-pyridine (24 mmol, 5.1 g) in MeOH (15 mL) was hydrogenated in the presence of 500 mg palladium (10% on activated carbon). After stirring at r.t. for 20 h the reaction mixture was filtered through Celite 521. The filter aid was washed with MeOH several times. The filtrate was evaporated to dryness to afford 4-pyridin-4-ylmethyl-phenylamine (1.84 g) as a grey solid. Anal. RP-HPLC indicated a single product. A solution of this product in MeOH (15 mL) was cooled on an ice bath and was treated first with HCl (conc. 1.75 mL) followed by addition of cyanamide (50% aq soln.; 5 mL). The reaction mixture was heated at reflux for 18 h. The solvent was evaporated and the residue was washed with EtOAc/MeOH (2:1, v/v) to afford N-(4-pyridin-4-ylmethyl-phenyl)-guanidine hydrochloride (2.25 g) as a white solid.

A mixture of 3-dimethylamino-1-(2,4-dimethyl-thiazol-5-yl)-propenone (1 mmol, 0.21 g) and N-(4-pyridin-4-ylmethyl-phenyl)-guanidine hydrochloride (2 mmol, 0.40 mg) in 2-methoxylethanol was treated with NaOH (40 mg). The reaction mixture was allowed to heat at reflux for 2 d. The solvent was evaporated and the residue was crystallised from EtOAc/MeOH to afford the title compound as an orange solid. ¹H-NMR (300 MHz, d₆-DMSO) δ 3.00 (s, 3H, CH₃), 3.02 (s, 3H, CH₃), 4.29 (s, 2H, CH₂), 7.44 (d, 1H, J=5.5 Hz, pyrimidinyl-H), 7.56 (m, 2H, Ph-H), 7.61 (m, 2H, Ar-H), 8.09 (m, 2H, Ph-H), 8.82 (m, 2H, Ar-H), 8.87 (d, 1H, J=5.5 Hz, pyrimidinyl-H). DE MALDI-TOF MS: [M+H]⁺=377.52 (C₂₁H₁₉N₅S requires 373.48).

### Example 20

{4-[4-(2,4-Dimethyl-thiazol-5-yl)-pyrimidin-2-ylamino]-phenyl}-trimethyl-ammonium [104]. A mixture of 3-dimethylamino-1-(2,4-dimethyl-thiazol-5-yl)-propenone (0.95 mmol, 0.19 g) and N-(4-dimethylamino-phenyl)-guanidine (2 mmol) in 2-methoxylethanol (5 mL) was added NaOH (40 mg). The reaction mixture was heated at 120° C. for 18 h. The solvent was evaporated and the residue was purified by chromatography, using EtOAc/PE to afford N,N-dimethyl-N′-[4-(2,4-dimethyl-thiazol-5-yl)-pyrimidin-2-yl]-benzene-1,4-diamine [103] (74 mg) as a reddish-brown solid. ¹H-NMR (300 MHz, d₆-DMSO) δ 2.62 (s, 3H, CH₃), 2.65 (s, 3H, CH₃), 2.86 (s, 6H, CH₃), 6.73 (m, 2H, Ph-H), 6.97 (d, 1H, J=5.1 Hz, pyrimidinyl-H), 7.56 (m, 2H, Ph-H), 8.44 (d, 1H, J=5.0 Hz, pyrimidinyl-H), 9.33 (s, 1H, NH). DE MALDI-TOF MS: [M+H]⁺=329.51 (C₁₇H₁₉N₅S requires 325.43).

To the above compound (0.13 mmol, 42 mg) in dry acetone (6 mL) was added 12 □L iodomethane dropwise and the reaction mixture was heated at reflux for 18 h. The solvent was evaporated and the resulting oil was triturated with toluene (5 mL). The resulting precipitate was filtered, washed with EtOAc and dried under high vacuum overnight to afford the title compound (18 mg). ¹H-NMR (300 MHz, d₆-DMSO) δ 2.63 (s, 3H, CH₃), 2.65 (s, 3H, CH₃), 3.56 (s, 9H, CH₃), 7.17 (d, 1H, J=5.4 Hz, pyrimidinyl-H), 7.88 (m, 2H, Ph-H), 7.96 (m, 2H, Ph-H), 8.57 (d, 1H, J=5.4 Hz, pyrimidinyl-H), 10.04 (s, 1H, NH). DE MALDI-TOF MS: [M+H]⁺=343.39 (C₁₉H₂₅N₅S requires 340.47).

### Example 21

[4-(2-Amino-4-methyl-thiazol-5-yl)-pyrimidin-2-yl]-(3-nitro-phenyl)-amine [105]. A mixture of thiourea (5.18 g, 0.068 mol) in dry MeOH (20 mL) was stirred and cooled on an ice bath. Pyridine (2 mL) was added, followed by 3-chloro-2,4-pentadione (9.15 g, 0.068 mol) dropwise. After completion of the addition the reaction mixture was allowed to warm to r. t. and stirring was continued for 4 h. The precipitates were filtered and washed with EtOAc to afford white solid 1-(2-amino-4-methyl-thiazol-5-yl)-ethanone.

A solution of this material (3.35 g, 0.021 mol) in N,N-dimethylformamide dimethylacetal (10 mL) was refluxed under N₂ for 4-6 h. The reaction mixture was evaporated to dryness. EtOAc was added to the residue and the precipitates were collected by filtration and were washed with EtOAc/PE (5:1, v/v) to afford N′-[5-(3-dimethylamino-acryloyl)-4-methyl-thiazol-2-yl]-N,N-dimethyl-formamidine as an orange solid (50-79%). ¹H-NMR (CDCl₃) δ: 2.64 (s, 3H, CH₃), 3.08 (s, 6H, CH₃), 3.11 (s, 6H, CH₃), 5.35 (d, 1H, J=12.2 Hz, CH), 7.67 (d, 1H, J=12.2 Hz, CH), 8.23 (s, 1H, N═CH). DE MALDI-TOF MS: [M+H]⁺=267.49 (C₁₂H₁₈N₆OS requires 266.36).

A mixture of this material (2.19 g, 8.2 mmol) and 3-nitrophenyl guanidine nitrate (2.00 g 8.2 mmol) in 2-methoxyethanol (10 mL) was treated with NaOH (0.33 g). After refluxing under N₂ for 20 h the reaction mixture was concentrated and purified by silica-gel chromatography using EtOAc/PE (7:1) to elute the title compound as a light-yellow solid (1.95 g, 72%), which was then recrystallised from EtOAc/MeOH. ¹H-NMR (DMSO-d₆) δ: 3.13 (s, 3H, CH₃), 7.02 (d, 1H, J=5.5 Hz, Py-H), 7.59 (m, 4H, Ph-H and NH₂), 7.82 (m, 1H, Ph-H), 8.16 (m, 1H, Ph-H), 8.44 (d, 1H, J=5.5 Hz, Py-H), 8.86 (br. s, 1H, NH).

### Example 22

The following compounds were prepared in a manner similar to that described in Example 21 above:

N-[4-(2-Amino-4-methyl-thiazol-5-yl)-pyrimidin-2-yl]-N′,N′-dimethyl-benzene-1,4-diamine [106]. Yellow solid; anal. RP-HPLC: t_{R}=9.83 min (0-60% MeCN in 0.1% aq CF₃COOH over 20 min, 1 mL/min, purity >95%). ¹H-NMR (CD₃OD) δ: 2.58 (s, 3H, CH₃), 3.28 (s, 6H, CH₃), 7.08 (d, 1H, J=5.5 Hz, pyrimidinyl-H), 7.56 (m, 2H, Ph-H), 7.89 (m, 2H, Ph-H), 8.45 (d, 1H, J=5.5 Hz, pyrimidinyl-H). MS (DE MALDI-TOF) m/z=326.0 [M+H]⁺ (C₁₆H₁₈N₆S requires 326.4).

[4-(2-Amino-4-methyl-thiazol-5-yl)-pyrimidin-2-yl]-(4-chloro-phenyl)-amine [107]. Brown solid; ¹H-NMR (DMSO-d₆) δ: 2.42 (s, 3H, CH₃), 6.88 (d, 1H, J=5.0 Hz, pyrimidinyl -H), 7.28 (m, 2H, Ph-H), 7.51 (br. s, 2H, NH2), 7.77 (m, 2H, Ph-H), 8.32 (d, 1H, J=5.1 Hz, pyrimidinyl-H), 9.56 (br. s, 1H, NH). MS (DE MALDI-TOF) m/z=318.4 [M+H]⁺ (C₁₄H₁₂ClN₅S requires 317.8).

[4-(2-Amino-4-methyl-thiazol-5-yl)-pyrimidin-2-yl]-(3-methoxy-phenyl)-amine [108]. Light yellow solid; ¹H-NMR (DMSO-d₆) δ: 2.41 (s, 3H, CH₃), 3.72 (s, 3H, CH₃), 6.50 (m, 1H, Ph-H), 6.88 (d, 1H, J=5.5Hz, pyrimidinyl-H), 7.14 (t, 1H, J=8.0 Hz, Ph-H), 7.30 (m, 1H, Ph-H), 7.47 (m, 1H, pyrimidinyl-H), 7.48 (br. s, 2H, NH2), 8.31 (d, 1H, J=5.5 Hz, pyrimidinyl-H), 9.41 (br. s, 1H, NH).

[4-(2-Amino-4-methyl-thiazol-5-yl)-pyrimidin-2-yl]-(3-fluoro-phenyl)-amine [109]. Grey solid; ¹H-NMR (DMSO-d₆) δ: 2.43 (s, 3H, CH₃), 6.71 (m, 1H, Ph-H), 6.92 (d, 1H, J=5.5 Hz, pyrimidinyl-H), 7.27 (m, 1H, Ph-H), 7.44 (m, 1H, Ph-H), 7.557 (br. s, 2H, NH2), 7.84 (m, 1H, Ph-H), 8.35 (d, 1H, J=5.5Hz, pyrimidinyl-H), 9.69 (sr. 1H, NH).

[4-(2-Amino-4-methyl-thiazol-5-yl)-pyrimidin-2-yl]-(4-trifluoromethyl-phenyl)-amine [110]. Brown solid; ¹H-NMR (DMSO-d₆) δ: 2.44 (s, 3H, CH₃), 6.96 (d, 1H, J=5.0 Hz, pyrimidinyl-H), 7.53 (br. s, 2H, NH2), 7.60 (d, 2H, J=9.0 Hz, Ph-H), 7.97 (d, 2H, J=8.5Hz, Ph-H), 8.38 (d, 1H, J=5.0 Hz, pyrimidinyl-H), 9.86 (br. s, H, NH). MS (DE MALDI-TOF) m/z=352.0 [M+H]⁺ (C₁₅H₁₂F₃N₅S requires 351.4).

[4-(2-Amino-4-methyl-thiazol-5-yl)-pyrimidin-2-yl]-(4-methoxy-phenyl)-amine [111]. Brown solid; ¹H-NMR (DMSO-d₆) δ: 2.41 (s, 3H, CH₃), 3.71 (s, 3H, CH₃), 6.80 (d, 1H, J=5.5 Hz, pyrimidinyl-H), 6.84 (m, 2H, Ph-H), 7.44 (br. s, 1H, NH), 7.63 (m, 2H, Ph-H), 8.26 (d, 1H, J=5.5 Hz, pyrimidinyl-H), and 9.20 (br. s, H, NH). MS (DE MALDI-TOF) m/z=312.9 [M+H]⁺ (C₁₅H₁₅N₅OS requires 313.4).

[4-(2-Amino-4-methyl-thiazol-5-yl)-pyrimidin-2-yl]-(3-chloro-phenyl)-amine [112]. Brown solid; ¹H-NMR (DMSO-d₆) δ: 2.43 (s, 3H, CH₃), 6.91 (d, 1H, J=5.5 Hz, pyrimidinyl-H), 6.94 (m, 1H, Ph-H), 7.26 (m, 1H, Ph-H), 7.55 (br. s 2H, NH₂), 7.64 (m, 1H, Ph-H), 8.02 (s, 1H, Ph-H), 8.34 (d, 1H, J=5.5 Hz, pyrimidinyl-H), 9.64 (br. s, 1H, NH).

[4-(2-Amino-4-methyl-thiazol-5-yl)-pyrimidin-2-yl]-(3-iodo-phenyl)-amine [113]. Dark solid; ¹H-NMR (DMSO-d₆) δ: 2.44 (s, 3H, CH₃), 6.90 (d, 1H, J=5.5 Hz, pyrimidinyl-H), 7.04 (t, 1H, J=7.5 Hz, Ph-H), 7.25 (m, 1H, Ph-H), 7.51 (br. s, 2H, NH₂), 7.65 (m, 1H, Ph-H), 8.26 (s, 1H, Ph-H), 8.34 (d, 1H, J=5.5 Hz, pyrimidinyl-H), 9.64 (br. s, 1H, NH). MS (DE MALDI-TOF) m/z=408.9 (C₁₄H₁₂IN₅S requires 409.3).

[4-(2-Amino-4-methyl-thiazol-5-yl)-pyrimidin-2-yl]-(4-iodo-phenyl)-amine [114]. Yellow solid; ¹H-NMR (DMSO-d₆) δ 2.48 (s, 3H, CH₃), 7.04 (d, 1H, J=5.5 Hz, pyrimidinyl-H), 7.59 (s, 2H, NH₂), 8.01 (m, 2H, Ph-H), 8.17 (m, 2H, Ph-H), 8.43 (d, 1H, J=5.5 Hz, pyrimidinyl-H), 10.27 (br. s, 1H, NH).

[4-(2-Amino-4-methyl-thiazol-5-yl)-pyrimidin-2-yl]-(4-fluoro-phenyl)-amine [115]. Grey solid; ¹H-NMR (DMSO-d₆) δ: 2.42 (s, 3H, CH₃), 6.86 (d, 1H, J=5.5 Hz, pyrimidinyl-H), 7.08 (m, 2H, Ph-H), 7.48 (br. s, 2H, NH₂), 7.74 (m, 2H, Ph-H), 8.30 (d, 1H, J=5.5 Hz, pyrimidinyl-H), 8.50, 9.42 (br. s 1H, NH). MS (DE MALDI-TOF) m/z=299.6 [M+H]⁺ (C₁₄H₁₂FN₅S requires 301.3).

3-[4-(2-Amino-4-methyl-thiazol-5-yl)-pyrimidin-2-ylamino]-phenol [116]. Dark-brown solid; ¹H-NMR (DMSO-D₆) δ: 2.41 (s, 3H, CH₃), 6.34 (m, 1H, Ph-H), 6.84 (d, 1H, J=5.5 Hz, pyrimidinyl-H), 7.01 (m, 1H, Ph-H), 7.19 (s, 1H, Ph-H), 7.23 (m, 1H, Ph-H), 7.48 (br. s, 2H, NH₂), 8.29 (d, 1H, J=5.5 Hz, pyrimidinyl-H), 9.26 (br. s, 2H, NH & OH).

[4-(2-Amino-4-methyl-thiazol-5-yl)-pyrimidin-2-yl]-(4-iodo-3-nitro-phenyl)-amine [117]. Dark solid; anal. RP-HPLC: t_{R}=15.5 min (0-60% MeCN in 0.1% aq CF₃COOH over 20 min, 1 mL/min, purity >95%). ¹H-NMR (DMSO-d₆) δ: 2.48 (s, 3H, CH₃), 6.92 (d, 1H, J=5.5 Hz, pyrimidinyl-H), 7.37 (m, 1H, Ph-H), 7.82 (m, 1H, Ph-H), 8.19 (m, 1H, Ph-H), 8.36 (d, 1H, J=5.5 Hz, pyrimidinyl-H), 8.68 (br. s, 2H, NH2), 9.86 (br. s, 1H, NH).

2-{4-[4-(2-Amino-4-methyl-thiazol-5-yl1-pyrimidin-2-ylamino]-phenyl}-ethanol [118]. Light yellow solid; anal. RP-HPLC: t_{R}=10.9 min (0-60% MeCN in 0.1% aq CF₃COOH over 20 min, 1 mL/min, purity >95%). ¹H-NMR (DMSO-d₆) δ: 2.85 (s, 3H, CH₃), 3.04 (t, 2H, J=7.32 Hz, CH₂), 3.94 (t, 2H, J=7.32 Hz, CH₂), 7.35 (d, 1H, J=5.5 Hz, pyrimidinyl-H), 7.50 (d, 2H, J=8.5Hz, Ph-H), 7.96 (d, 2H, J=8.5 Hz, Ph-H), 8.76 (d, 1H, J=5.5 Hz, pyrimidinyl-H), 8.68 (br. s, 2H, NH₂), 9.12 (br. s, 2H, NH & OH).

[4-(2-Amino-4-methyl-thiazol-5-yl)-pyrimidin-2-yl]-(3-bromo-phenyl)-amine [119]. Yellow solid; ¹H-NMR (DMSO-d₆) δ: 2.44 (s, 3H, CH₃), 6.91 (d, 1H, J=5.4Hz, Py-H), 7.08 (m, 1H, Ph-H), 7.20 (m, 1H, Ph-H), 7.53 (m, 1H, Ph-H), 7.68 (m, 1H, Ph-H), 8.15 (br. s, 2H, NH₂), 8.35 (d, 1H, J=5.4 Hz, pyrimidinyl-H), 9.62 (br. s 1H, NH). MS (DE MALDI-TOF) m/z=362.2 (C₁₄H₁₂BrN₅S requires 362.3).

[4-(2-Amino-4-methyl-thiazol-5-yl)-pyrimidin-2-yl]-(4-bromo-phenyl)-amine [120]. Brown solid; ¹H-NMR (DMSO-d₆) δ: 2.43 (s, 3H, CH₃), 6.89 (d, 1H, J=5.5 Hz, pyrimidinyl-H), 7.42 (m, 2H, Ph-H), 7.47 (br. s, 2H, NH₂), 7.73 (m, 2H, Ph-H), 8.33 (d, 1H, J=5.5 Hz, pyrimidinyl-H), 9.57 (br. s, 1H, NH). MS (DE MALDI-TOF) m/z=362.2 (C₁₄H₁₂BrN₅S requires 362.3).

[4-(2-Amino-4-methyl-thiazol-5-yl)-pyrimidin-2-yl]-(4-chloro-3-trifluoromethyl-phenyl)-amine [121]. Brown solid; ¹H-NMR (DMSO-d₆) δ: 2.43 (s, 3H, CH₃), 6.96 (d, 1H, J=5.6 Hz, pyrimidinyl-H), 7.76 (m, 2H, Ph-H/NH), 8.00 (m, 1H, Ph-H), 8.38 (m, 2H, Py-H/Ph-H), 9.89 (br. s, 1H, NH). MS (DE MALDI-TOF) m/z=388.8 [M+H]⁺ (C₁₅H₁₁ClF₃N₅S requires 385.8).

### Example 23

N-[4-(2, 4-Dimethyl-thiazol-5-yl)-pyrimidin-2-yl]-N′,N′-dimethyl-benzene-1,4-diamine [103]. A solution of 1-(2,4-dimethyl-thiazol-5-yl)-ethanone (10 g, 0.06 mol) in of N,N-dimethylformamide dimethylacetal (10 mL) was refluxed under N₂. After 18 h, the reaction mixture was evaporated to dryness in vacuo. The resulting solid material was crystallised from a minimum amount of isopropyl ether/CH₂Cl₂ to afford 9.94 g 3-dimethylamino-1-(2,4-dimethyl-thiazol-5-yl)-propenone as a brown powder (79%). ¹H-NMR (CDCl₃) δ: 2.66 (s, 6H, CH₃), 2.70 (s, 6H, CH₃), 5.37 (d, 1H, J=12.2 Hz, CH), 7.66 (d, 1H, J=12.2 Hz, CH).

To a solution of this compound (0.21 g, 1.0 mmol) and N-(4-dimethylamino-phenyl)-guanidine nitrate (50 mg) (prepared from N,N-dimethyl-benzene-1,4-diamine and cyanamide) in 2-methoxylethanol (3 mL) was added NaOH (80 mg). The reaction mixture was refluxed for 8 h. The solvent was evaporated in vacuo and the residue was purified by SiO₂ flash chromatography (EtOAc) to afford 2-[N-(4-N,N-dimethylaminophenyl)]-4-(2,4-dimethylthiazol-5-yl)-pyrimidineamine as a yellow solid (26 mg, 79%). RP-HPLC: t_{R}=11.2 min (0-60% MeCN in 0.1% aq CF₃COOH over 20 min, 1 mL/min, purity >95%). ¹H-NMR (DMSO-d₆) δ: 2.60 (s, 3H, CH₃), 2.62 (s, 3H, CH₃), 2.82 (s, 6H, CH₃), 6.70 (d, 2H, J=8.8 Hz, Ph-H), 6.95 (d, 1H, J=5.3 Hz, pyrimidinyl-H), 7.53 (d, 2H, J=8.9 Hz, Ph-H), 8.40 (d, 1H, J=5.3 Hz, pyrimidinyl-H), 9.26 (br. s, 1H, NH). MS (ESI+) m/z=326.2 [M+H]⁺ (C₁₇H₁₉N₅S requires 325.4).

### Example 24

The following compounds were prepared in a manner analogous to that described in Example 23 above:

N¹-[4-(2,4-Dimethyl-thiazol-5-yl)-pyrimidin-2-yl]-4-[β-(phenoxy)-triethylamine]-amine [122]. Buff-coloured solid; ¹H-NMR (CD₃OD) δ: 1.11 (t, 6H, J=7.3 Hz, CH₃), 2.66 (s, 3H, CH₃), 2.68 (s, 3H, CH₃), 2.70 (q, 4H, J=7.1 Hz, CH₂), 2.93 (t, 2H, J=5.6 Hz, CH₂), 4.10 (t, 2H, J=5.9 Hz, CH₂), 6.91 (d, 2H, J=9.3Hz, Ph-H), 6.99 (d, 1H, J=5.4Hz, pyrimidinyl-H), 7.56 (d, 2H, J=9.3 Hz, Ph-H), 8.37 (d, 1H, J=5.1 Hz, pyrimidinyl-H). MS (DE MALDI-TOF) m/z=397.2 [M+H]⁺ (C₂₁H₂₇N₅OS requires 397.5).

2-{4-[4-(2,4-Dimethyl-thiazol-5-yl)-pyrimidin-2-ylamino]-phenyl}-ethanol [123]. Light yellow solid; anal. RP-HPLC: t_{R}=13.1 min (0-60% MeCN in 0.1% aq CF₃COOH over 20 min, 1 mL/min, purity >95%). ¹H-NMR (DMSO-d₆) δ: 2.89 (s, 3H, CH₃), 3.07 (m, 2H, CH₂), 3.98 (t, 2H, J=7.5 Hz, CH₂), 7.46 (d, 1H, J=5.5 Hz, pyrimidinyl-H), 7.55 (d, 2H, J=8.5 Hz, Ph-H), 8.06 (d, 2H, J=8.5 Hz, Ph-H), 8.90 (d, 1H, J=5.5 Hz, pyrimidinyl-H). MS (ESI⁺) m/z=326.7 (C₁₇H₁₈N₄OS requires 326.4).

2-({4-[4-(2,4-Dimethyl-thiazol-5-yl)-pyrimidin-2-ylamino]-phenyl}-ethyl-amino)-ethanol [124]. Yellow solid; ¹H-NMR (CDCl₃) δ: 1.08 (t, 3H, J=7.1 Hz, CH₃), 2.61 (s, 3H, CH-₃), 2.64 (s, 3H, CH₃), 3.34 (q, 2H, J=7.1 Hz, CH₂), 3.46 (br. s, 1H, OH), 6.36 (t, 2H, J=5.9 Hz, CH₂), 6.70 (t, 2H, J=5.4 Hz, CH₂), 6.76 (d, 2H, J=9.0Hz, Ph-H), 6.79 (d, 1H, J=5.1 Hz, pyrimidinyl-H), 6.84 (br. s, 1H, NH), 7.39 (d, 2H, J=9.0 Hz, Ph-H), 8.30 (d, 1H, J=5.1 Hz, pyrimidinyl-H).

(3,4-Dimethoxy-phenyl)-[4-(2,4-dimethyl-thiazol-5-yl)-pyrimidin-2-yl]-amine [125]. Brown solid; ¹H-NMR (CDCl₃) δ: 2.69 (s, 3H, CH₃), 2.70 (s, 3H, CH₃), 3.89 (s, 3H, CH₃), 3.95 (s, 3H, CH₃), 6.87 (d, 1H, J=8.5Hz, Ph-H), 6.92 (d, 1H, J=5.1 Hz, pyrimidinyl-H), 7.04 (dd, 1H, J=8.5, 2.2 Hz, Ph-H), 7.14 (br. s, 1H, NH), 7.36 (m, 1H, Ph-H), 8.38 (d, 1H, J=5.4 Hz, pyrimidinyl-H).

5-[4-(2,4-Dimethyl-thiazol-5-yl)-pyrimidin-2-ylamino]-2-methoxy-phenol [126]. Yellow solid; ¹H-NMR (DMSO-d₆) δ: 2.61 (s, 3H, CH₃), 2.63 (s, 3H, CH₃), 3.72 (s, 3H, CH₃), 6.83 (d, 1H, J=8.8 Hz, Ph-H), 6.99 (d, 1H, J=5.4 Hz, pyrimidinyl-H), 7.15-7.19 (m, 2H, Ph-H, NH), 8.44 (d, 1H, J=5.6 Hz, pyrimidinyl-H), 8.82 (br. s, 1H, OH), 9.34 (d, 1H, J=1.5 Hz, Ph-H).

### Example 25

N⁴-[4-(2, 4-Dimethyl-thiazol-4-yl)-pyrimidin-2-yl]-N¹,N¹-dimethyl-2-nitro-benzene-1,4-diamine [127]. HNO₃ (69% aq, 24 μL, 0.36 mmol) was added dropwise to Ac₂O (1 mL) at room temperature, keeping the internal temperature below 25° C. The mixture was stirred at room temperature for 15 min before cooling to −5° C. in an ice-MeOH bath. Compound N-[4-(2,4-dimethyl-thiazol-5-yl)-pyrimidin-2-yl]-N′,N′-dimethyl-benzene-1,4-diamine (50 mg, 0.15 mmol) was slurried in Ac₂O (1 mL) and added dropwise to the cooled solution of acetyl nitrate. The mixture was stirred with cooling for 1 h then a further 2 h at room temperature. The mixture was poured into ice-water (20 mL) and the pH was adjusted to 7-8 by addition of saturated aq NaHCO₃ solution. The mixture was extracted with EtOAc. The combined organics were washed with brine, dried on MgSO₄, and filtered. The solvent was evaporated in vacuo to give a dark solid, which was purified by flash chromatography, eluted with heptane/EtOAc to afford 32 mg of the title compound as a pale reddish solid. RP-HPLC: t_{R}=12.7 min (10-70% MeCN in 0.1% aq CF₃COOH over 20 min, 1 mL/min, purity >95%). ¹H-NMR (DMSO-d₆): δ 2.62 (s, 3H, CH₃), 2.64 (s, 3H, CH₃), 2.74 (s, 6H, CH₃), 7.09 (d, 1H, J=5.1 Hz, pyrimidinyl-H), 7.23 (d, 1H, J=8.8 Hz, Ph-H), 7.77 (dd, 1H, J=8.7, 2.7 Hz, Ph-H), 8.39 (d, 1H, J=2.7 Hz), 8.51 (d, 1H, J=5.1Hz, pyrimidinyl-H), 9.81 (br. s, 1H, NH).

In an alternative preparation: 4-Fluoro-3-nitro-aniline (20 g, 128 mmol) was dissolved in EtOH (300 mL) and dimethylamine (5.6 M solution in EtOH, 360 mL, 2.02 mol) was added in a steady stream. After refluxing for 18 h, the reaction mixture was cooled and 100 mL water was added. EtOH was removed by evaporation and the residue was extracted with Et₂O (3×100 mL). The combined organics were washed with brine, filtered, and the solvent was evaporated to afford 22.8 g of 4-(dimethylamino)-3-nitroaniline as a black oil. This was dissolved in EtOH (80 mL) and HNO₃ (69% aq, 18.5 mL, 22.1 mmol) added dropwise followed by cyanamide (50% wt in water, 37 mL, 476 mmol). The mixture was heated at reflux for 18 h. Once cooled, the mixture was poured into Et₂I (1 L). The ethereal supernatant was decanted and the residue was treated with propan-2-ol, followed by Et₂O to give 19.0 g of the corresponding guanidine nitrate as a tan solid. This was stirred with K₂CO₃ (15.04 g, 108.8 mmol) in 2-methoxyethanol (250 mL) for 10 min before adding 3-dimethylamino-1-(2,4-dimethylthiazol-5-yl)-propenone (9.53 g, 45.33 mmol). The mixture was heated at 125° C. for 18 h. The reaction mixture was concentrated and diluted with EtOAc, filtered through a pad of silica and evaporated to give a dark oil, which was purified by chromatography, using EtOAc to elute the title product as a reddish solid. Recrystallisation from toluene yielded 7.3 g pure title compounds.

### Example 26

2-[N-(4-N,N-Dimethylamino-3-chlorophenyl)]-4-(2,4-dimethylthiazol-5-yl)-pyrimidineamine [128]. A solution of 3-chloro-4-fluoronitrobenzene (3.0 g, 17.1 mmol), dimethylamine hydrochloride (1.53 g, 18.8 mmol) and K₂CO₃ (4.96 g, 35.9 mmol) in Me₂SO (20 mL) was heated in a sealed tube at 105° C. for 18 h. On cooling the reaction mixture was poured into water (200 mL) and extracted with EtOAc. The combined organics were washed with brine, dried on MgSO₄, filtered, and evaporated to give 3.47 g of 3-chloro-4-(dimethylamino) nitrobenzene as a yellow solid. An aliquot of this (3.4 g, 16.95 mmol) was dissolved in 20 mL of EtOH/AcOH (1:1, v/v) with warming. Iron powder (−325 mesh, 9.5 g, 170 mmol) was added in small portions. The mixture was then heated on a steam bath for 30 min. The mixture was cooled, filtered through a pad of celite and the filtrate was evaporated to give 3.33 g of 3-chloro-4-(dimethylamino)aniline as a dark solid. A solution of this compound in EtOH (10 mL) was treated with HNO₃ (69% aq, 2.6 mL, 40.6 mmol) dropwise, followed by cyanamide (50% solution in water, 5.3 mL, 67.78 mmol). After heating for 18 h at reflux the reaction mixture was cooled to room temperature, poured into Et₂O (100 ml) and basified with NaOH solution (2 N, 100 mL). The ethereal layer was separated. The aqueous phase was extracted with Et₂O. The combined organic phases were washed with brine, dried on MgSO₄, filtered, and evaporated to give a black oil, which solidified on standing to afford 1.6 g of the title compound. RP-HPLC: t_{R}=12.7 min (10-70% MeCN in 0.1% aq CF₃COOH over 20 min, 1 mL/min, purity >95%). ¹H-NMR CD₃OD) δ: 2.68 (s, 3H, CH₃), 2.70 (s, 3H, CH₃), 2.75 (s, 6H, CH₃), 7.05 (d, 1H, J=5.1 Hz), 7.15 (d, 1H, J=8.8 Hz, pyrimidinyl-H), 7.49 (dd, 1H, J=8.8, 2.4 Hz, Ph-H), 7.94 (d, 1H, J=2.4 Hz, Ph-H), 8.43 (d, 1H, J=5.4 Hz, pyimidinyl-H). MS (ESI⁺) m/z=393 [M+Na] (C₁₇H₁₈N₆O₂S requires 370.4).

### Example 27

The following compounds were prepared in a manner analogous to that described in Example 26 above:

N⁴-[4-(2,4-Dimethyl-thiazol-5-yl)-pyrimidin-2-yl]-N¹,N¹-dimethyl-2-trifluoromethyl-benzene-1,4-diamine [129]. Off-white solid; ¹H-NMR (CDCl₃) δ: 2.62 (s, 3H, CH₃), 2.64 (s, 9H, CH₃), 6.91 (d, 1H, J=5.5 Hz), 7.16 (br. s, 1H, NH), 7.31 (d, 1H, J=8.5 Hz, pyrimidinyl-H), 7.63 (dd, 1H, J=9.0, 2.5 Hz, Ph-H), 7.94 (d, 1H, J=2.5 Hz, Ph-H), 8.36 (d, 1H, J=5.0 Hz, pyrimidinyl-H).

N¹-[4-(2,4-Dimethyl-thiazol-5-yl)-pyrimidin-2-yl]-4-methoxy-N³,N³-dimethyl-benzene-1,3-diamine [130]. Off-white solid; ¹H-NMR (CDCl₃) δ: 2.58 (s, 3H, CH₃), 2.62 (s, 3H, CH₃), 2.67 (s, 6H, CH₃), 3.74 (s, 3H, CH₃), 6.84 (d, 1H, J=8.5 Hz, pyrimidinyl-H), 6.98 (d, 1H, J=5.0 Hz, pyrimidinyl-H), 7.33 (m, 1H, Ph-H), 8.44 (d, 1H, J=5.0 Hz, pyrimidinyl-H), 9.33 (br. s, 1H, NH).

### Example 28

N,N-Dimethyl-N′-[4-(4-methyl-2-methylamino-thiazol-5-yl)-pyrimidin-2-yl]-benzene-1,4-diamine [131]. A solution of 3-chloro-2,4-pentanone (2.5 g, 19 mmol) in MeOH (15 mL) was treated with N-methyl-2-thiourea (1.67 g, 19 mmol) and pyridine (15 mL). After stirring at room temperature for 3 h the resulting precipitates were filetered and washed with Et₂O to afford of 1-(4-methyl-2-methylamino-thiazol-5-yl)-ethanone (2.05 g) as a white solid. Without further purification this compound was treated with of N,N-dimethylformamide dimethylacetal (10 mL_ at 100-110° C. for 22 h. The reaction mixture was concentrated and the precipitate was collected and washed with EtOAc to afford 3-dimethylamino-1-(4-methyl-2-methylaminothiazol-5-yl)-propenone as an orange solid. ¹H-NMR (CDCl₃) δ: 2.55 (s, 3H, CH₃), 2.94 (s, 3H, CH₃), 3.40 (s, 6H, CH₃), 5.29 (d, 1H, J=12.2 Hz, CH), 7.62 (d, 1H, J=12.2 Hz, CH).

The title compounds was then obtained by condensation of 3-dimethylamino-1-(4-methyl-2-methylaminothiazol-5-yl)-propenone and N-(4-dimethylamino-phenyl)-guanidine nitrate as usual. Dark-brown solid; anal. RP-HPLC: t_{R}=10.2 min (0-60% MeCN in 0.1% aq CF₃COOH over 20 min, 1 mL/min, purity >95%). ¹H-NMR (DMSO-d₆) δ: 2.62 (s, 3H, CH₃), 3.31 (s, 6H, CH₃), 7.11 (d, 1H, J=5.5 Hz, pyrimidinyl-H), 7.53 (m, 2H, Ph-H), 7.88 (m, 2H, Ph-H), 8.44 (d, 1H, J=5.5 Hz, pyrimidinyl-H), 8.68 (br. s, 1H, NH).

The following compound was obtained in an analogous manner:

(4-Iodo-3-nitro-phenyl)-[4-(4-methyl-2-methylamino-thiazol-5-yl)-pyrimidin-2-yl]-amine [132]. Dark-brown solid; ¹H-NMR (DMSO-d₆) δ: 2.49 (s, 3H, CH₃), 3.24 (s, 3H, CH₃), 6.96 (d, 1H, J=6.0 Hz, pyrimidinyl-H), 7.37 (d, 1H, J=8.0 Hz, Ph-H), 7.82 (m, 1H, Ph-H), 8.36 (d, 1H, J=6.0 Hz, pyrimidinyl-H), 8.68 (s, 1H, Ph-H), 9.86 (br. s, 1H, NH).

### Example 29

[4-(2-Ethylamino-4-methyl-thiazol-5-yl)-pyrimidin-2-yl]-(3-nitro-phenyl)-amine [133]. 3-Dimethylamino-1-(2-ethylamino-4-methyl-thiazol-5-yl)-propenone was prepared by reaction between 1-(2-ethylamino-4-methyl-thiazol-5-yl)-ethanone and 3-chloro-pentane-2,4-dione. It was then condensed with N-(3-nitro-phenyl)-guanidine nitrate in the usual manner to afford the title compound. Yellow solid; ¹H-NMR (DMSO-d₆) δ: 1.14 (m, 3H, CH₃), 2.47 (s, 3H, CH₃), 3.23 (m, 2H, CH₂), 6.99 (d, 1H, J=5.0 Hz, pyrimidinyl-H), 7.55 (m, 1H, Ph-H), 7.77 (m, 1H, Ph-H), 8.02 (m, 1H, Ph-H), 8.39 (d, 1H, J=5.0 Hz, pyrimidinyl-H), 8.47 (s, 1H, Ph-H), 9.98 (br. s, 1H, NH).

### Example 30

The following compounds were prepared in a manner analogous to that described in Example 29 above:

[4-(2-Ethylamino-4-methyl-thiazol-5-yl)-pyrimidin-2-yl]-(4-trifluoromethyl-phenyl)-amine [135]. Brown solid; ¹H-NMR (DMSO-d₆) δ: 1.16 (t, 3H, J=7.0 Hz, CH₃), 2.46 (s, 3H, CH₃), 3.27 (m, 2H, CH₂), 6.98 (d, 1H, J=5.5 Hz, pyrimidinyl-H), 7.60 (d, 2H, J=9.0 Hz, Ph-H), 7.97 (d, 2H, J=9.0 Hz, Ph-H), 8.14 (br. s, 1H, NH), 8.37 (d, 1H, J=5.5 Hz, pyrimidinyl-H), 9.86 (br. s, 1H, NH).

[4-(2-Ethylamino-4-methyl-thiazol-5-yl)-pyrimidin-2-yl]-(3-methoxy-phenyl)-amine [136]. Brown solid; ¹H-NMR (DMSO-d₆) δ: 1.17 (m, 3H, CH₃), 2.48 (s, 3H, CH₃), 3.25 (m, 2H, CH₂), 6.49 (m, 1H, Ph-H), 6.89 (d, 1H, J=5.5 Hz, pyrimidinyl-H), 7.14 (t, 1H, J=8.5 Hz, Ph-H), 7.26 (m, 1H, Ph-H), 7.52 (m, 1H, Ph-H), 8.31 (d, 1H, J=5.5 Hz, pyrimidinyl-H), 8.49 (br. s, 1H, NH), 9.39 (br. s, 1H, NH).

(3-Chloro-phenyl)-[4-(2-ethylamino-4-methyl-thiazol-5-yl)-pyrimidin-2-yl]-amine [137]. Brown solid; ¹H-NMR (DMSO-d₆) δ: 1.15 (m, 3H, CH₃), 2.47 (s, 3H, CH₃), 3.22 (m, 2H, CH₂), 6.94 (m, 2H, Ph-H & pyrimidinyl-H), 7.26 (t, 1H, J=9.0 Hz, Ph-H), 7.58 (m, 1H, Ph-H), 8.10 (m, 1H, Ph-H), 8.35 (d, 1H, J=5.5 Hz, pyrimidinyl-H), 9.65 (br. s, 1H, NH).

[4-(2-Ethylamino-4-methyl-thiazol-5-yl)-pyrimidin-2-yl]-(4-methyl-3-nitro-phenyl)-amine [138]. Brown solid; ¹H-NMR (DMSO-d₆) δ: 1.19 (t, 3H, J=7.5 Hz, CH₃), 2.49 (s, 3H, CH₃), 3.24 (m, 2H, CH₂), 6.95 (d, 1H, J=5.5 Hz, pyrimidinyl-H), 7.37 (d, 1H, J=8.5 Hz, Ph-H), 7.81(m, 1H, Ph-H), 8.35 (d, 1H, J=5.5 Hz, pyrimidinyl-H), 8.66 (s, 1H, Ph-H), 9.83 (br. s, 1H, NH).

(4-Chloro-phenyl)-[4-(2-ethylamino-4-methyl-thiazol-5-yl)-pyrimidin-2-yl]-amine [147]. Brown solid; ¹H-NMR (DMSO-d₆) δ: 1.16 (m, 3H, CH₃), 2.45 (s, 3H, CH₃), 3.24 (m, 2H, CH₂), 6.90 (d, 1H, J=5.0 Hz, pyrimidinyl-H), 7.30 (d, 2H, J=9.0 Hz, Ph-H), 7.79 (d, 2H, J=9.0 Hz, Ph-H), 8.32 (d, 1H, J=5.0 Hz, pyrimidinyl-H), 9.57 (sbr, 1H, NH).

### Example 31

[4-(2-Butylamino-4-methyl-thiazol-5-yl)-pyrimidin-2-yl]-(4-fluoro-phenyl)-amine [139]. The titled compound was prepared by condensation of 1-(2-butylamino-4-methyl-thiazol-5-yl)-3-dimethylamino-propenone with 4-fluorophenylguanidine nitrate in the usual manner to afford the title compound. Grey solid; ¹H-NMR (DMSO-d₆) δ: 0.90 (m, 3H, CH₃), 1.33 (m, 2H, CH₂), 1.53 (m, 2H, CH₂), 2.48 (s, 3H, CH₃), 3.22 (m, 2H, CH₂), 6.87 (d, 1H, J=5.0 Hz, pyrimidinyl-H), 7.10 (m, 2H, Ph-H), 7.74 (m, 2H, Ph-H), 8.11 (br. s, 1H, NH), 8.30 (d, 1H, J=5.5 Hz, pyrimidinyl-H), 9.42 (br. s, 1H, NH).

### Example 32

[4-(2-Dimethylamino-4-methyl-thiazol-5-yl)-pyrimidin-2-yl]-(3-nitro-phenyl)-amine [140]. A mixture of 1-(4-methyl-2-methylamino-thiazol-5-yl)-ethanone (0.40 g, 2.4 mmol) in THF (2 mL) was treated with NaH (0.113 g, 4.7 mmol). After heating at 40° C. for 0.5 h MeI (0.35 g, 2.4 mmol) was added. Heating was continued for a further 2 h. After cooling, the solution was diluted with EtOAc, washed with brine, and dried over MgSO₄. The solvent was evaporated to afford 1-(2-dimethylamino-4-methyl-thiazol-5-yl)-ethanone as a yellow solid. ¹H-NMR (CDCl₃) δ: 2.36 (s, 3H, CH₃), 2.51 (s, 3H, CH₃), 3.10 (s, 6H, CH₃).

The above compound was heated in of N,N-dimethylformamide dimethylacetal (2 mL) at 125° C. for 4 h. The reaction mixture was concentrated and the residue was purified by SiO₂ chromatography (EtOAc/MeOH, 95:5) to afford the desired product 3-dimethylamino-1-(2-dimethylamino-4-methyl-thiazol-5-yl)-propenone. ¹H-NMR (CDCl₃) δ: 2.49 (s, 6H, CH₃), 3.03 (s, 6H, CH₃), 3.29 (s, 3H, CH₃), 5.23 (d, 1H, J=12.0 Hz, CH), 7.51 (d, 1H, J=12.0 Hz, CH). Condensation of this compound with N-(3-nitro-phenyl)-guanidine nitrate in the usual manner afforded the titled compound as a brown solid. ¹H-NMR (DMSO-d₆) δ: 3.12 (s, 9H, CH₃), 7.02 (d, 1H, J=5.0 Hz, pyrimidinyl-H), 7.55 (t, 1H, J=8.0 Hz, Ph-H), 7.77 (m, 1H, Ph-H), 7.93 (m, 1H, Ph-H), 8.41 (d, 1H, J=6.0 Hz, pyrimidinyl-H), 8.49 (s, 1H, Ph-H), 9.10 (br. s, 1H, NH).

### Example 33

The following compounds were prepared in a manner analogous to that described in Example 34 above:

(4-Chloro-phenyl)-[4-(2-dimethylamino-4-methyl-thiazol-5-yl)-pyrimidin-2-yl]-amine [141]. Brown solid; ¹H-NMR (DMSO-d₆) δ: 3.09 (s, 9H, CH₃), 6.93 (d, 1H, J=5.5 Hz, pyrimidinyl-H), 7.32 (d, 2H, J=9.5 Hz, Ph-H), 7.79 (d, 2H, J=9.5 Hz, Ph-H), 8.33 (d, 1H, J=5.0 Hz, pyrimidinyl-H), 9.57 (br. s, 1H, NH).

[4-(2-Dimethylamino-4-methyl-thiazol-5-yl)-pyrimidin-2-yl]-(4-fluoro-phenyl)-amine [142]. Grey solid; ¹H-NMR (DMSO-d₆) δ: 3.08 (s, 9H, CH₃), 6.89 (d, 1H, J=5.0 Hz, pyrimidinyl-H), 7.11 (m, 2H, Ph-H), 7.74 (m, 2H, Ph-H), 8.31 (d, 1H, J=5.5 Hz, pyrimidinyl-H), 9.44 (br. s, 1H, NH).

(3-Chloro-phenyl)-[4-(2-dimethylamino-4-methyl-thiazol-5-yl)-pyrimidin-2-yl]-amine [143]. Brown solid; ¹H-NMR (DMSO-d₆) δ: 3.10 (s, 9H, CH₃), 6.96 (d, 2H, pyrimidinyl-H & Ph-H), 7.27 (t, 1H, J=8.0 Hz, Ph-H), 7.52 (m, 1H, Ph-H), 8.20 (s, 1H, Ph-H), 8.37 (d, 1H, J=5.5 Hz, pyrimidinyl-H), 9.71 (br. s, 1H, NH).

### Example 34

2-{4-Methyl-5-[2-(3-nitro-phenylamino)-pyrimidin-4-yl]-thiazol-2-ylamino}-ethanol [144]. To a mixture of [4-(2-amino-4-methyl-thiazol-5-yl)-pyrimidin-2-yl]-(3-nitro-phenyl)-amine (0.33 g, 1.0 mmol) and iodoethanol (0.44 g, 2.6 mmol) in dry DMF (2 mL) was added tert-butylimino-2-diethylamino-1,3-dimethylperhydro-1,3-diazaphosphorine (0.5 mL). The reaction mixture was heated at 124° C. for 20 h. The product was isolated as a brown solid by preparative RP-HPLC (Vydac 218TP1022, 9 mL/min) using a gradient from 10-70% MeCN in 0.1% aq CF₃COOH over 40 min. Anal. RP-HPLC: t_{R}=14.30 min (Vydac 218TP54, 0-60% MeCN in 0.1% aq CF₃COOH over 20 min, 1 mL/min, 25° C., purity>97%). ¹H-NMR (CD₃OD) δ: 3.30 (s, 3H, CH₃), 3.91 (t, 2H, J=4.6 Hz, CH₂), 4.25 (t, 2H, J=4.6Hz, CH₂), 7.21(d, 1H, J=5.2 Hz, pyrimidinyl-H), 7.54 (m, 1H, Ph-H), 7.89 (m, 2H, Ph-H), 8.59 (d, 1H, J=5.2 Hz, pyrimidinyl-H), 8.90 (s, 1H, Ph-H).

2-{5-[2-(4-Fluoro-phenylamino)-pyrimidin-4-yl]-4-methyl-thiazol-2-ylamino}-ethanol [145]. This compound was prepared from [4-(2-amino-4-methyl-thiazol-5-yl)-pyrimidin-2-yl]-(4-fluoro-phenyl)-amine in a manner analogous to that described for compound [58]. ¹H-NMR (DMSO-d₆) δ: 2.44 (s, 3H, CH₃), 3.54 (m, 2H, CH₂), 4.78 (m, 2H, CH₂), 6.87 (d, 1H, J=5.2 Hz, pyrimidinyl-H), 7.09 (m, 2H, Ph-H), 7.75 (m, 2H, Ph-H), 8.30 (d, 1H, J=5.2 Hz, pyrimidinyl-H), 8.11 (m, 1H, NH), 9.43 (s, 1H, NH). DE MALDI-TOF MS: [M+H]⁺=345.79 (C₁₆H₁₆FN₅OS requires 345.40).

### Example 35

5-[2-(4-Hydroxy-phenylamino)-pyrimidin-4-yl]-3,4-dimethyl-3H-thiazol-2-one [152] To an ice-cooled solution of potassium thiocyanate (5.67 g, 58 mmol) in Me₂CO (45 mL) was added 3-chloro-pentane-2,4-dione (6.95 mL, 58 mmol) dropwise. After completion of the addition the reaction mixture was warmed to room temperature and stirred for a further 6 h. The solvent was evaporated to dryness. The residue was dissolved in EtOH (30 mL) and HCl (conc. aq, 15 mL) was added. The mixture was heated to reflux for 14 h. It was concentrated and the precipitate was collected, washed with cold MeOH and then Et₂O to afford 9.1 g of a pale solid. This compound was treated with N,N-dimethylformamide dimethylacetal (13 mL) at 100-110° C. for 8 h. The reaction mixture was concentrated and the residue was purified by SiO₂ flash chromatography (EtOAc/PE) to afford 5-(3-dimethylamino-acryloyl)-3,4-dimethyl-3H-thiazol-2-one. ¹H-NMR (CDCl₃) δ: 2.50 (s, 3H, CH₃), 3.07 (s, 3H, CH₃), 3.21 (s, 6H, CH₃), 5.09 (d, 1H, J=12.0 Hz, CH), 7.59 (d, 1H, J=12.0 Hz, CH).

A solution of 5-(3-dimethylamino-acryloyl)-3,4-dimethyl-3H-thiazol-2-one (0.23 g, 1.0 mmol) in of 2-methoxylethanol (3 mL) was treated with N-(4-hydroxy-phenyl)-guanidine nitrate (0.42 g, 2.0 mmol). After refluxing for 20 h the reaction mixture was concentrated and purified by SiO₂ flash chromatography (EtOAc). Recrystallisation from EtOAc afforded the tilted compound (25 mg) as brown crystals. Anal. RP-HPLC: t_{R}=11.8 min (0-60% MeCN in 0.1% aq CF₃COOH over 20 min, 1 mL/min, purity >95%). ¹H-NMR (DMSO-d₆) δ: 2.52 (s, 3H, CH₃), 3.27 (s, 3H, CH₃), 6.68 (d, 2H, J=8.9 Hz, Ph-H), 6.81 (d, 1H, J=5.5 Hz, pyrimidinyl-H), 7.44 (d, 2H, J=8.9 Hz, Ph-H), 8.34 (d, 1H, J=5.5 Hz, pyrimidinyl-H), 9.12 (br. s, 1H, OH/NH), 9.24 (br. s, 1H, NH/OH).

### Example 36

The following compounds were prepared in a similar manner to the procedures described above:

3,4-Dimethyl-5-[2-(3-nitro-phenylamino)-pyrimidin-4-yl]-3H-thiazol-2-one [153] Brown crystals. Anal. RP-HPLC: t_{R}=17.8 min (0-60% MeCN in 0.1% aq CF₃COOH over 20 min, 1 mL/min, purity >97%). ¹H-NMR (DMSO-d₆) δ: 2.42 (s, 3H, CH₃), 3.16 (s, 3H, CH₃), 6.92 (d, 1H, J=5.0 Hz, pyrimidinyl-H), 7.42 (d, 1H, J=8.0 Hz, Ph-H) 7.65 (m, 1H, Ph-H), 7.88 (m, 1H, Ph-H), 8.37 (d, 1H, J=5.0Hz, pyrimidinyl-H), 8.72 (br. s, 1H, NH).

5-[2-(4-Iodo-phenylamino)-pyrimidin-4-yl]-3,4-dimethyl-3H-thiazol-2-one [154] Brown solid; anal. RP-HPLC: t_{R}=18.8 min (0-60% MeCN in 0.1% aq CF₃COOH over 20 min, 1 mL/min, purity >95%). ¹H-NMR (DMSO-d₆) δ: 2.83 (s, 3H, CH₃), 3.59 (s, 3H, CH₃), 7.24 (d, 1H, J=5.0 Hz, pyrimidinyl-H), 7.87 (m, 4H, Ph-H), 8.71 (d, 1H, J=5.0 Hz, pyrimidinyl-H). ¹³C-NMR (DMSO-d₆) δ: 14.96, 30.30, 85.01, 109.42, 109.41, 110.32, 121.93, 137.69, 137.70, 138.74, 140.89, 158.55, 159.24, 159.93, 170.39.

5-[2-(4-Fluoro-phenylamino)-pyrimidin-4-yl]-3,4-dimethyl-3H-thiazol-2-one [155] Gray solid; ¹H-NMR (DMSO-d₆) δ: 2.92 (s, 3H, CH₃), 3.67 (s, 3H, CH₃), 7.32 (d, 1H, J=5.0 Hz, pyrimidinyl-H), 7.51 (m, 2H, Ph-H), 8.11 (m, 2H, Ph-H), 8.80 (d, 1H, J=5.0 Hz, pyrimidinyl-H).

5-[2-(4-Chloro-phenylamino)-pyrimidin-4-yl]-3,4-dimethyl-3H-thiazol-2-one [156] Light yellow solid; ¹H-NMR (DMSO-d₆) δ: 2.55 (s, 3H, CH₃), 3.29 (s, 3H, CH₃), 6.97 (d, 1H, J=5.0 Hz, pyrimidinyl-H), 7.32 (d, 2H, J=8.5 Hz, Ph-H), 7.76 (d, 2H, J=9.0 Hz, Ph-H), 8.44 (d, 1H, J=5.0 Hz, pyrimidinyl-H), 9.75 (br. s, 1H, NH).

5-[2-(4-Methoxy-phenylamino)-pyrimidin-4-yl]-3, 4-dimethyl-3H-thiazol-2-one [157] Light yellow solid; ¹H-NMR (DMSO-d₆) δ: 2.54 (s, 3H, CH₃), 3.28 (s, 3H, CH₃), 3.71 (s, 3H, CH₃), 6.86 (m, 3H, pyrimidinyl-H & Ph-H), 7.59 (d, 2H, J=9.0 Hz, Ph-H), 8.37 (d, 1H, J=5.0 Hz, pyrimidinyl-H), 9.39 (br. s, 1H, NH).

5-[2-(3-Hydroxy-phenylamino)-pyrimidin-4-yl]-3,4-dimethyl-3H-thiazol-2-one [158] Light yellow solid; anal. RP-HPLC: t_{R}=15.4 min (0-60% MeCN in 0.1% aq VF₃COOH over 20 min, 1 mL/min, purity >95%). ¹H-NMR (DMSO-d₆) δ: 2.55 (s, 3H, CH₃), 3.26 (s, 3H, CH₃), 6.36 (m, 1H, Ph-H), 6.90 (d, 1H, J=5.5 Hz, pyrimidinyl-H), 7.03 (t, 1H, J=8.5 Hz, Ph-H), 7.16 (m, 1H, Ph-H), 7.22 (s, 1H, Ph-H), 8.40 (d, 1H, J=5.5 Hz, pyrimidinyl-H), 9.39 (br. s, 1H, NH).

5-[2-(4-Fluoro-3-nitro-phenylamino)-pyrimidin-4-yl]-3,4-dimethyl-3H-thiazol-2-one [159] Brown solid; ¹H-NMR (DMSO-d₆) δ: 2.42 (s, 3H, CH₃), 2.81 (s, 3H, CH₃), 6.36 (m, 1H, Ph-H), 6.91 (d, 1H, J=5.5 Hz, pyrimidinyl-H), 7.31 (m, 1H, Ph-H), 8.33 (m, 1H, Ph-H), 8.48 (d, 1H, J=5.5 Hz, pyrimidinyl-H), 8.52 & 9.68 (br. s, 1H, NH).

5-[2-(4-Chloro-3-methyl-phenylamino)-pyrimidin-4-yl]-3, 4-dimethyl-3H-thiazol-2-one [160] Yellow solid; ¹H-NMR (DMSO-d₆) δ: 2.30 (s, 3H, CH₃), 2.55 (s, 3H, CH₃), 3.27 (s, 3H, CH₃), 6.96 (d, 1H, J=5.5 Hz, pyrimidinyl-H), 7.30 (d, 1H, J=9.0 Hz, Ph-H), 7.52 (m, 1H, Ph-H), 7.81 (m, 1H, Ph-H), 8.43 (d, 1H, J=5.5 Hz, pyrimidinyl-H), 9.69 (br. s, 1H, NH).

5-[2-(3-Iodo-4-methyl-phenylamino)-pyrimidin-4-yl]-3,4-dimethyl-3H-thiazol-2-one [161] Brown solid; ¹H-NMR (DMSO-d₆) δ: 2.28 (s, 3H, CH₃), 3.30 (s, 3H, CH₃), 6.96 (d, 1H, J=5.0 Hz, pyrimidinyl-H), 7.14 (m, 1H, Ph-H), 7.21 (m, 1H, Ph-H), 7.53 (m, 1H, Ph-H), 8.42 (d, 1H, J=5.0 Hz, pyrimidinyl-H), 9.65 (br. s, 1H, NH).

5-[2-(4-Fluoro-3-methyl-phenylamino)-pyrimidin-4-yl]-3,4-dimethyl-3H-thiazol-2-one [162] Grey solid; ¹H-NMR (DMSO-d₆) δ: 2.21 (s, 3H, CH₃), 2.55 (s, 3H, CH₃), 3.26 (s, 3H, CH₃), 6.92 (d, 1H, J=5.0 Hz, pyrimidinyl-H), 7.04 (t, 1H, J=9.0 Hz, Ph-H), 7.48 (m, 1H, Ph-H), 7.68 (m, 1H, Ph-H), 8.40 (d, 1H, J=5.5 Hz, pyrimidinyl-H), 9.54 (br. s, 1H, NH).

3,4-Dimethyl-5-[2-(4-methyl-3-nitro-phenylamino)-pyrimidin-4-yl]-3H-thiazol-2-one [163] Yellow solid; ¹H-NMR (DMSO-d₆) δ: 2.44 (s, 3H, CH₃), 2.55 (s, 3H, CH₃), 3.27 (s, 3H, CH₃), 7.03 (d, 1H, J=5.0 Hz, pyrimidinyl-H), 7.40 (t, 1H, J=8.5 Hz, Ph-H), 7.84 (m, 1H, Ph-H), 8.48 (d, 1H, J=5.0 Hz, pyrimidinyl-H), 8.59 (s, 1H, Ph-H), 9.99 (br. s, 1H, NH).

5-[2-(4-Dimethylamino-phenylamino)-pyrimidin-4-yl]-3,4-dimethyl-3H-thiazol-2-one [164] Yellow solid; anal. RP-HPLC: t_{R}=19.6 min (0-60% MeCN in 0.1% aq CF₃COOH over 20 min, 1 mL/min, purity >95%). ¹H-NMR (DMSO-d₆) δ: 2.83 (s, 3H, CH₃), 2.90 (s, 6H, CH₃), 3.08 (s, 3H, CH₃), 6.73 (m, 2H, Ph-H), 6.81(d, 1H, J=5.5 Hz, pyrimidinyl-H), 7.03 (m, 1H, Ph-H), 7.50 (m, 1H, Ph-H), 8.32 (d, 1H, J=5.5 Hz, pyrimidinyl-H), 9.24 (br. s, 1H, NH).

The biological activity of the compounds of the invention was demonstrated by measuring the CDK inhibition by virtue of an assay-based screen, and/or by a cytotoxicity assay using one or more cell lines.

### Example 37

Kinase Specificity of Selected Compound

Selected compounds from the above examples were investigated for their kinase selectivity. A panel of protein kinases, including the CDKs relevant to the present invention, as well as a representative number of functionally unrelated kinases, were used.

Assays for CDK4/Cyclin D1, CDK2/Cyclin E, CDIK1/Cyclin B kinase may be carried out by monitoring phosphorylation of GST-Rb in an appropriate system. Thus, GST-Rb phosphorylation, induced by CDK4/Cyclin D1, CDK2/Cyclin E or CDK1/Cyclin B is determined by incorporation of radio-labeled phosphate in GST-Rb(772-928) using radiolabelled ATP in 96-well format in vitro kinase assay. The phosphorylation reaction mixture (total volume 40 μl) consisted of 50 mM HEPES pH 7.4, 20 mM MgCl₂, 5 mM EGTA, 2 mM DTT, 20 mM β-glycerophosphate, 2 mM NaF, 1 mM Na₃VO₄, Protease Inhibitors Cocktail (Sigma, see above), BSA 0.5 mg/ml, 1 μg purified enzyme complex, 10 μl of GST-Rb-Sepharose beads, 100 μM ATP, 0.2 μCi ³²P-ATP. The reaction is carried out for 30 min at 30° C. at constant shaking. At the end of this period 100 μl of 50 mM HEPES, pH 7.4 and 1 mM ATP is added to each well and the total volume transferred onto GFC filtered plate. The plate is washed 5 times with 200 μl of 50 mM HEPES, pH 7.4 and 1 mM ATP. To each well were added 50 μl scintillant liquid and the radioactivity of the samples is measured on Scintilation counter (Topcount, HP). The IC50 values of different peptides were calculated using GraFit software.

Alternatively, CDK2/cyclin A kinase assays may be performed in 96-well plates using recombinant CDK2/cyclin A. Assay buffer consisted of 25 mM β-glycerophosphate, 20 mM MOPS, 5 mM EGTA, 1 mM DTT, 1 mM NaVO₃, pH 7.4, into which is added 2-4 μg of CDK2/cyclin A with substrate pRb(773-928). The reaction is initiated by addition of Mg/ATP mix (15 mM MgCl₂, 100 μM ATP with 30-50 kBq per well of [γ-³²P]-ATP) and mixtures incubated for 10-30 min, as required, at 30° C. Reactions were stopped on ice, followed by filtration through p81 filterplates (Whatman Polyfiltronics, Kent, UK). After washing 3 times with 75 mM orthophosphoric acid, plates were dried, scintillant added and incorporated radioactivity measured in a scintillation counter (TopCount, Packard Instruments, Pangboume, Berks, UK).

PKCα kinase activity may be measured by the incorporation of radio-labeled phosphate in Histone 3, as described. The reaction mixture (total volume 65 μl) consist of 50 mM Tris-HCl, 1 mM Calcium acetate, 3 mM DTT, 0.03 mg/ml Phosphatidylserine, 2.4 μg/ml PMA, 0.04% NP40, 12 mM Mg/Cl, purified PKCα-100 ng, Histone 3, 0.2 mg/ml, 100 μM ATP, 0.2 μCi [γ-³²P]-ATP. The reaction is carried over 15 min at 37° C. in microplate shaker and is stopped by adding 10 μl 75 mM orthophosphoric acid and placing the plate on ice. 50 μl of the reaction mixture is transferred onto P81 filterplate and after washing off the free radioactive phosphate (3 times with 200 μl 75 mM orthophosphoric acid per well) 50 μl of scintillation liquid (Microscint 40) were added to each well and the radioactivity is measured on Scintillation counter (Topcount, HP).

For use in said assays CDK2 and/or PKC may be obtained from available sources or produced by recombinant methods as described. His-tagged CDK2/Cyclin E and CDK1/Cyclin B may be co-expressed and PKCα singularly expressed in Sf 9 insect cells infected with the appropriate baculovirus constructs. The cells are harvested two days after infection by low speed centrifugation and the proteins purified from the insect cell pellets by Metal-chelate chromatography. Briefly, the insect cell pellet is lysed in Buffer A (10 mM Tris-HCl, pH 8.0, 150 mM NaCl, 0.02% NP40 and 5 mM β-marcaptoethanol, 1 mM NaF. 1 mM Na₃VO₄ and Protease Inhibitors Coctail (Sigma) containing AEBSF, pepstatin A, E 64, bestatin, leupeptin) by sonication. The soluble fraction is cleared by centrifugation and loaded onto Ni-NTA-Agarose (Quiagen). Non bound proteins were washed off with 300 mM NaCl, 5-15 mM Imidazole in Buffer A and the bound proteins eluted with 250 mM Imidazole in Buffer A. The purified proteins are extensively dialyzed against Storage buffer (20 mM HEPES pH 7.4, 50 mM NaCl, 2 mM DTT, 1 mM EDTA, 1 mM EGTA, 0.02% NP40, 10% v/v Glycerol) aliquoted and stored at −70° C. PKC-α-6×His may be purified the same way but using different buffers—50 mM NaH2PO4, pH 8.0 and 0.05% Triton X-100 instead of Tris and NP40 respectively.

The results in the Table 2 below show that the compounds in question exhibit a high degree of selectivity for inhibition of CDKs. Further results for CDK inhibition are shown below in Tables 3 and 4.

### Example 38

CDK 7 and 9 Assays

CTD peptide substrate (biotinyl-Ahx-(Tyr-Ser-Pro-Thr-Ser-Pro-Ser)₄-NH₂; 1-2 mg/mL) and recombinant human CDK7/cyclin H, CDK9/cyclin T1, or CDK9/cyclin K (0.5-2 μg) were incubated for 45 min at 30° C. in the presence of varying amounts of test compound in 20 mM MOPS pH 7.2, 25 mM β-glycerophosphate, 5 mM EGTA, 1 mM DTT, 1 mM sodium vanadate, 15 mM MgCl₂, and 100 μM ATP (containing a trace amount of ³²PγATP) in a total volume of 25 μL in a 96-well microtiter plate. The reaction was stopped by placing the plate on ice for 2 min. Avidin (50 μg) was added to each well, and the plate was incubated at room temp for 30 min. The samples were transferred to a 96-well P81 filter plate, and washed (4×200 μL per well) with 75 mM phosphoric acid. Microscint 40 scintillation liquid (50 μL) was added to each well, and the amount of ³²P incorporation for each sample was measured using a Packard Topcount microplate scintillation counter.

The results are shown above in Tables 2, 3 and 4.

### Example 39

Anti-HIV Efficacy Evaluation in Fresh Human PBMCs

Representative compounds of the present invention were tested for antiviral activity against HIV-1 in human peripheral blood mononuclear cells (PBMCs) using the clinical paediatric HIV strains RoJo or WeJo. PBMCs were cultured under conditions which promote cell survival and HIV replication. Antiviral activity was tested for from 6-9 log₁₀ serial dilutions of a 100 μM compound stock solution in DMSO. The following parameters were derived: IC₅₀ and IC₉₀ (concentrations inhibiting virus replication by 50 and 90%, respectively, TC₅₀ (concentration decreasing cell viability by 50%), and TI (therapeutic index: TC₅₀ /IC₅₀).

Fresh PBMCs, seronegative for HIV and HBV, were isolated from screened donors (Interstate Blood Bank, Inc. Memphis, Tenn.). Cells were pelleted/washed 2-3 times by low speed centrifugation and re-suspension in PBS to remove contaminating platelets. The Leukophoresed blood was then diluted with Dulbecco's Phosphate Buffered Saline (DPBS) and layered over Lymphocyte Separation Medium (LSM; Cellgro® by Mediatech, Inc.; density 1.078±0.002 g/mL; Cat.# 85-072-CL) in a 50 mL centrifuge tube and then centrifuged. Banded PBMCs were gently aspirated from the resulting interface and subsequently washed with PBS by low speed centrifugation. After the final wash, cells were enumerated by trypan blue exclusion and re-suspended in RPMI 1640 supplemented with fetal bovine serum (FBS), and L-glutamine, Phytohemagglutinin (PHA-P, Sigma). The cells were allowed to incubate at 37° C. After incubation, PBMCs were centrifuged and resuspended in RPMI 1640 with FBS, L-glutamine, penicillin, streptomycin, gentamycin, and recombinant human IL-2 (R&D Systems, Inc). IL-2 is included in the culture medium to maintain the cell division initiated by the PHA mitogenic stimulation. PBMCs were maintained in this with bi-weekly medium changes until used in the assay protocol. Cells were kept in culture for a maximum of two weeks before being deemed too old for use in assays and discarded. Monocytes were depleted from the culture as the result of adherence to the tissue culture flask.

For the standard PBMC assay, PHA-P stimulated cells from at least two normal donors were pooled, diluted and plated in the interior wells of a 96-well round bottom microplate. Pooling of mononuclear cells from more than one donor was used to minimise the variability observed between individual donors, which results from quantitative and qualitative differences in HIV infection and overall response to the PHA and IL-2 of primary lymphocyte populations. Each plate contained virus/cell control wells (cells plus virus), experimental wells (drug plus cells plus virus) and compound control wells (drug plus media without cells, necessary for MTS monitoring of cytotoxicity). Since HIV-1 is not cytopathic to PBMCs, this allows the use of the same assay plate for both antiviral activity and cytotoxicity measurements. Test drug dilutions were prepared in microtiter tubes and each concentration was placed in appropriate wells using the standard format. A predetermined dilution of virus stock was placed in each test well (final MOI≅0.1). The PBMC cultures were maintained for seven days following infection at 37° C., 5% CO₂. After this period, cell-free supernatant samples were collected for analysis of reverse transcriptase activity and/or HIV p24 content. Following removal of supernatant samples, compound cytotoxicity was measured by addition of MTS to the plates for determination of cell viability. Wells were also examined microscopically and any abnormalities were noted.

Reverse Transcriptase Activity Assay

A microtiter plate-based reverse transcriptase (RT) reaction was utilised (Buckheit et al., AIDS Research and Human Retroviruses 7:295-302, 1991). Tritiated thymidine triphosphate (³H-TTP, 80 Ci/mmol, NEN) was received in 1:1 dH₂O:Ethanol at 1 mCi/mL. Poly rA:oligo dT template:primer (Pharmacia) was prepared as a stock solution, followed by aliquoting and storage at −20° C. The RT reaction buffer was prepared fresh on a daily basis. The final reaction mixture was prepared by combining ³H-TTP, dH₂O, poly rA:oligo dT stock and reaction buffer. This reaction mixture was placed in a round bottom microtiter plate and supernatant containing virus was added and mixed. The plate was incubated at 37° C. for 60 minutes. Following incubation, the reaction volume was spotted onto DE81 filter-mats (Wallac), in a sodium phosphate buffer or 2×SSC (Life Technologies). Next they were washed in distilled water, in 70% ethanol, and then dried. Incorporated radioactivity (counts per minute, CPM) was quantified using standard liquid scintillation techniques.

The results for selected compounds of the invention are shown below in Table 5.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1

| No. | Structure | Name |
|---|---|---|
| 1 | 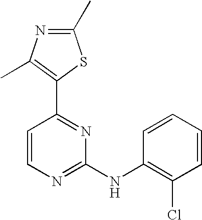 | (2-Chloro-phenyl)-[4-(2,4-dimethyl- thiazol-5-yl)-pyrimidin-2-yl]-amine |
| 2 | 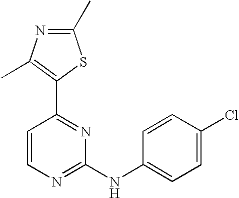 | (4-Chloro-phenyl)-[4-(2,4-dimethyl- thiazol-5-yl)-pyrimidin-2-yl]-amine |
| 3 | 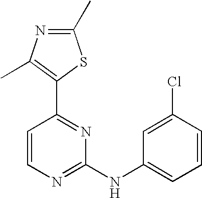 | (3-Chloro-phenyl)-[4-(2,4-dimethyl- thiazol-5-yl)-pyrimidin-2-yl]-amine |
| 4 | 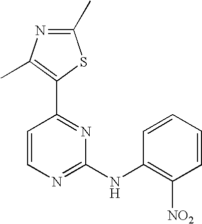 | [4-(2,4-Dimethyl-thiazol-5-yl)-pyrimidin-2- yl]-(2-nitro-phenyl)-amine |
| 5 | 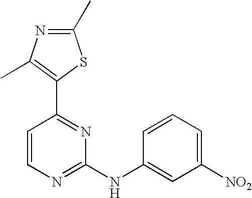 | [4-(2,4-Dimethyl-thiazol-5-yl)-pyrimidin-2- yl]-(3-nitro-phenyl)-amine |
| 6 | 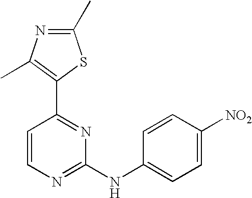 | [4-(2,4-Dimethyl-thiazol-5-yl)-pyrimidin-2- yl]-(4-nitro-phenyl)-amine |
| 7 | 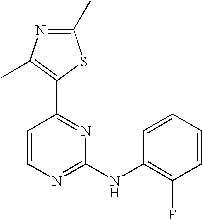 | [4-(2,4-Dimethyl-thiazol-5-yl)-pyrimidin-2- yl]-(2-fluoro-phenyl)-amine |
| 8 | 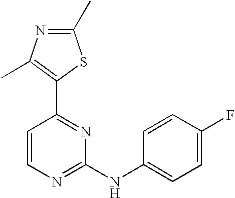 | [4-(2,4-Dimethyl-thiazol-5-yl)-pyrimidin-2- yl]-(4-fluoro-phenyl)-amine |
| 9 | 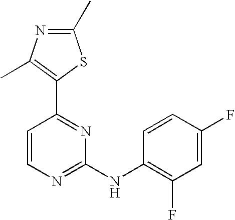 | (2,4-Difluoro-phenyl)-[4-(2,4-dimethyl- thiazol-5-yl)-pyrimidin-2-yl]-amine |
| 10 | 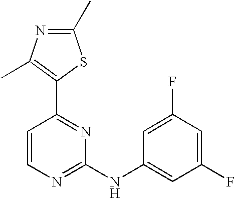 | (3,5-Difluoro-phenyl)-[4-(2,4-dimethyl- thiazol-5-yl)-pyrimidin-2-yl]-amine |
| 11 | 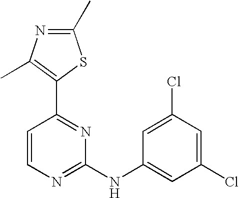 | (3,5-Dichloro-phenyl)-[4-(2,4-dimethyl- thiazol-5-yl)-pyrimidin-2-yl]-amine |
| 12 | 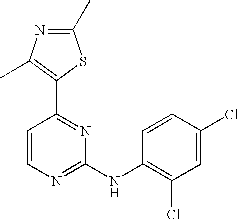 | (2,4-Dichloro-phenyl)-[4-(2,4-dimethyl- thiazol-5-yl)-pyrimidin-2-yl]-amine |
| 13 | 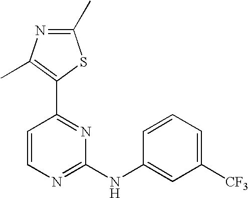 | [4-(2,4-Dimethyl-thiazol-5-yl)-pyrimidin-2- yl]-(3-trifluoromethyl-phenyl)-amine |
| 14 | 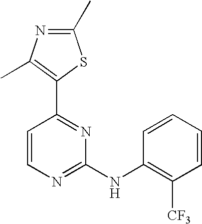 | [4-(2,4-Dimethyl-thiazol-5-yl)-pyrimidin-2- yl]-(2-trifluoromethyl-phenyl)-amine |
| 15 | 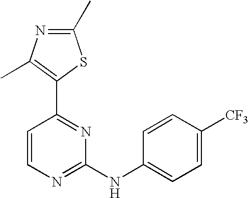 | [4-(2,4-Dimethyl-thiazol-5-yl)-pyrimidin-2- yl]-(4-trifluoromethyl-phenyl)-amine |
| 16 | 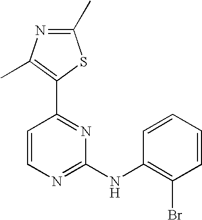 | (2-Bromo-phenyl)-[4-(2,4-dimethyl- thiazol-5-yl)-pyrimidin-2-yl]-amine |
| 17 | 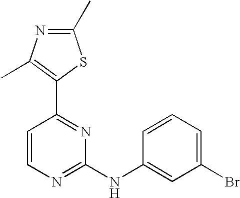 | (3-Bromo-phenyl)-[4-(2,4-dimethyl- thiazol-5-yl)-pyrimidin-2-yl]-amine |
| 18 | 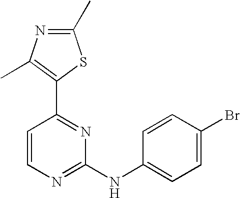 | (4-Bromo-phenyl)-[4-(2,4-dimethyl- thiazol-5-yl)-pyrimidin-2-yl]-amine |
| 19 | 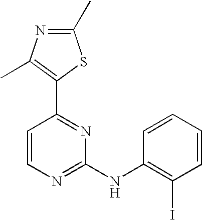 | [4-(2,4-Dimethyl-thiazol-5-yl)-pyrimidin-2- yl]-(2-iodo-phenyl)-amine |
| 20 | 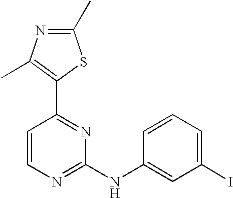 | [4-(2,4-Dimethyl-thiazol-5-yl)-pyrimidin-2- yl]-(3-iodo-phenyl)-amine |
| 21 | 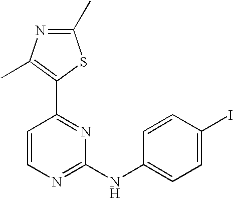 | [4-(2,4-Dimethyl-thiazol-5-yl)-pyrimidin-2- yl]-(4-iodo-phenyl)-amine |
| 22 | 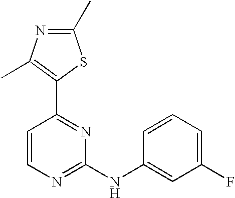 | [4-(2,4-Dimethyl-thiazol-5-yl)-pyrimidin-2- yl]-(3-fluoro-phenyl)-amine |
| 23 | 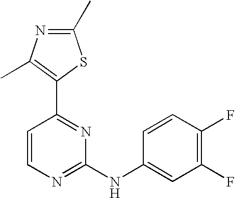 | (3,4-Difluoro-phenyl)-[4-(2,4-dimethyl- thiazol-5-yl)-pyrimidin-2-yl]-amine |
| 24 | 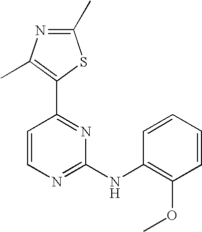 | [4-(2,4-Dimethyl-thiazol-5-yl)-pyrimidin-2- yl]-(2-methoxy-phenyl)-amine |
| 25 | 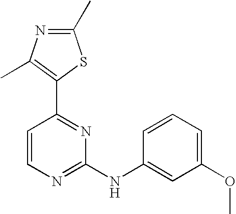 | [4-(2,4-Dimethyl-thiazol-5-yl)-pyrimidin-2- yl]-(3-methoxy-phenyl)-amine |
| 26 | 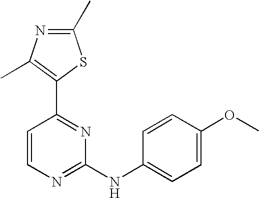 | [4-(2,4-Dimethyl-thiazol-5-yl)-pyrimidin-2- yl]-(4-methoxy-phenyl)-amine |
| 27 | 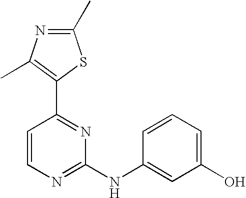 | 3-[4-(2,4-Dimethyl-thiazol-5-yl)-pyrimidin- 2-ylamino]-phenol |
| 28 | 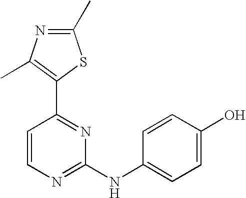 | 4-[4-(2,4-Dimethyl-thiazol-5-yl)-pyrimidin- 2-ylamino]-phenol |
| 29 | 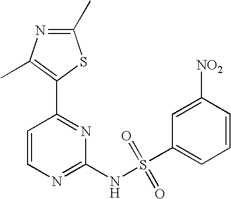 | N-[4-(2,4-Dimethyl-thiazol-5-yl)- pyrimidin-2-yl]-3-nitro- benzenesulfonamide |
| 30 | 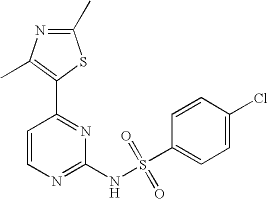 | 4-Chloro-N-[4-(2,4-dimethyl-thiazol-5-yl)- pyrimidin-2-yl]-benzenesulfonamide |
| 31 | 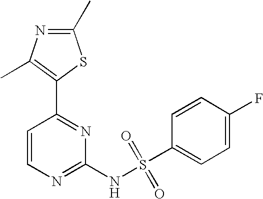 | N-[4-(2,4-Dimethyl-thiazol-5-yl)- pyrimidin-2-yl]-4-fluoro- benzenesulfonamide |
| 32 | 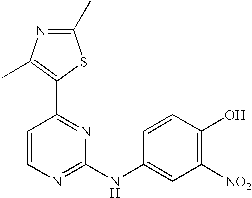 | 4-[4-(2,4-Dimethyl-thiazol-5-yl)-pyrimidin- 2-ylamino]-2-nitro-phenol |
| 33 | 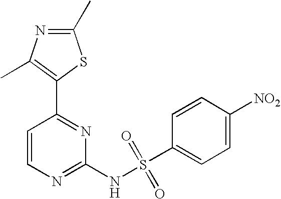 | N-[4-(2,4-Dimethyl-thiazol-5-yl)- pyrimidin-2-yl]-4-nitro- benzenesulfonamide |
| 34 | 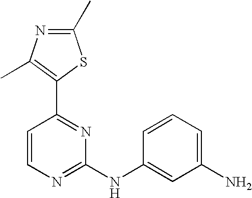 | N-[4-(2,4-Dimethyl-thiazol-5-yl)- pyrimidin-2-yl]-benzene-1,3-diamine |
| 35 | 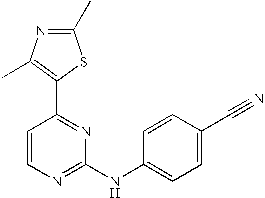 | 4-[4-(2,4-Dimethyl-thiazol-5-yl)-pyrimidin- 2-ylamino]-benzonitrile |
| 36 | 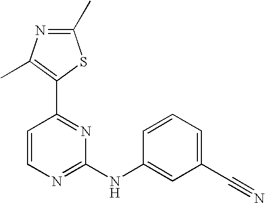 | 3-[4-(2,4-Dimethyl-thiazol-5-yl)-pyrimidin- 2-ylamino]-benzonitrile |
| 37 | 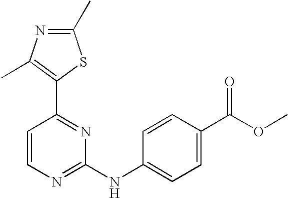 | 4-[4-(2,4-Dimethyl-thiazol-5-yl)-pyrimidin- 2-ylamino]-benzoic acid methyl ester |
| 38 | 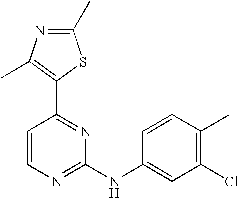 | (3-Chloro-4-methyl-phenyl)-[4-(2,4- dimethyl-thiazol-5-yl)-pyrimidin-2-yl]- amine |
| 39 | 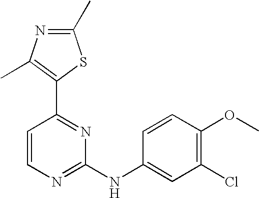 | (3-Chloro-4-methoxy-phenyl)-[4-(2,4- dimethyl-thiazol-5-yl)-pyrimidin-2-yl]- amine |
| 40 | 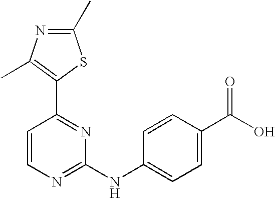 | 4-[4-(2,4-Dimethyl-thiazol-5-yl)-pyrimidin- 2-ylamino]-benzoic acid |
| 41 | 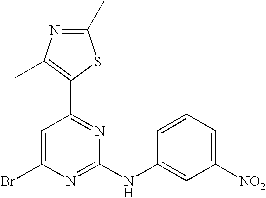 | [4-Bromo-6-(2,4-dimethyl-thiazol-5-yl)- pyrimidin-2-yl]-(3-nitro-phenyl)-amine |
| 42 | 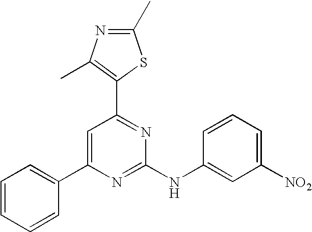 | [4-(2,4-Dimethyl-thiazol-5-yl)-6-phenyl- pyrimidin-2-yl]-(3-nitro-phenyl)-amine |
| 43 | 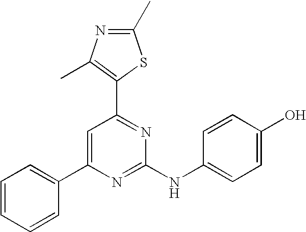 | 4-[4-(2,4-Dimethyl-thiazol-5-yl)-6-phenyl- pyrimidin-2-ylamino]-phenol |
| 44 | 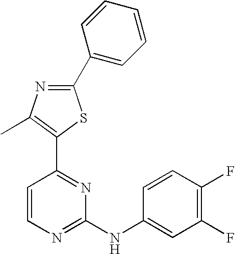 | (3,4-Difluoro-phenyl)-[4-(4-methyl-2- phenyl-thiazol-5-yl)-pyrimidin-2-yl]-amine |
| 45 | 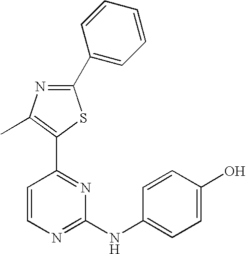 | 4-[4-(4-Methyl-2-phenyl-thiazol-5-yl)- pyrimidin-2-ylamino]-phenol |
| 46 | 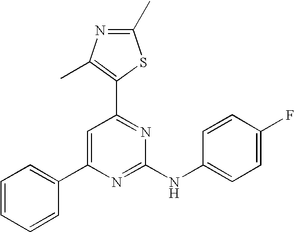 | [4-(2,4-Dimethyl-thiazol-5-yl)-6-phenyl- pyrimidin-2-yl]-(4-fluoro-phenyl)-amine |
| 47 | 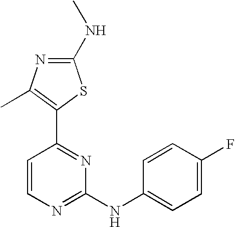 | (4-Fluoro-phenyl)-[4-(4-methyl-2- methylamino-thiazol-5-yl)-pyrimidin-2-yl]- amine |
| 48 | 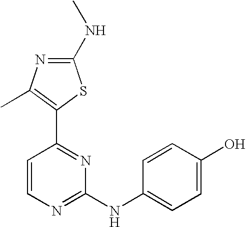 | 4-[4-(4-Methyl-2-methylamino-thiazol-5- yl)-pyrimidin-2-ylamino]-phenol |
| 49 | 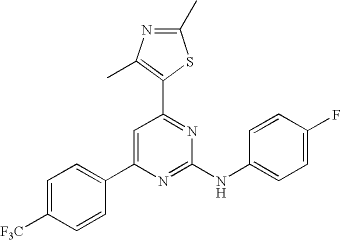 | [4-(2,4-Dimethyl-thiazol-5-yl)-6-(4- trifluoromethyl-phenyl)-pyrimidin-2-yl]-(4- fluoro-phenyl)-amine |
| 50 | 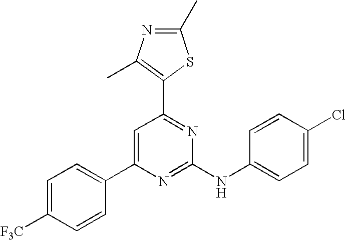 | (4-Chloro-phenyl)-[4-(2,4-dimethyl- thiazol-5-yl)-6-(4-trifluoromethyl-phenyl)- pyrimidin-2-yl]-amine |
| 51 | 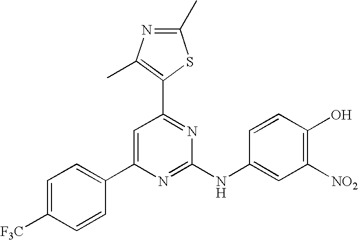 | 4-[4-(2,4-Dimethyl-thiazol-5-yl)-6-(4- trifluoromethyl-phenyl)-pyrimidin-2- ylamino]-2-nitro-phenol |
| 52 | 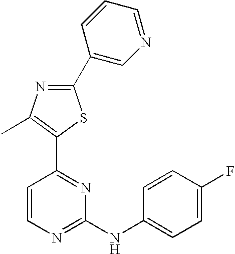 | (4-Fluoro-phenyl)-[4-(4-methyl-2-pyridin- 3-yl-thiazol-5-yl)-pyrimidin-2-yl]-amine |
| 53 | 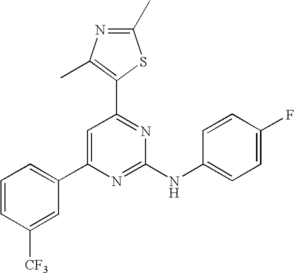 | [4-(2,4-Dimethyl-thiazol-5-yl)-6-(3- trifluoromethyl-phenyl)-pyrimidin-2-yl]-(4- fluoro-phenyl)-amine |
| 54 | 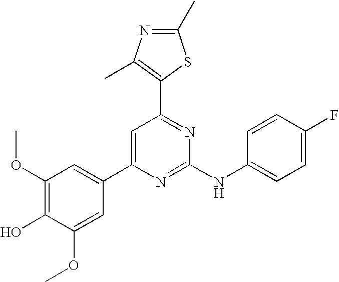 | 4-[6-(2,4-Dimethyl-thiazol-5-yl)-2-(4- fluoro-phenylamino)-pyrimidin-4-yl]-2,6- dimethoxy-phenol |
| 55 | 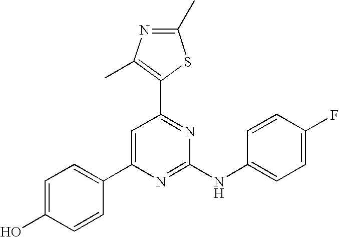 | 4-[6-(2,4-Dimethyl-thiazol-5-yl)-2-(4- fluoro-phenylamino)-pyrimidin-4-yl]- phenol |
| 56 | 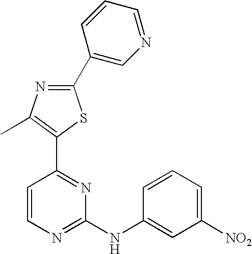 | [4-(4-Methyl-2-pyridin-3-yl-thiazol-5-yl)- pyrimidin-2-yl]-(3-nitro-phenyl)-amine |
| 57 | 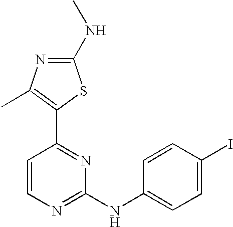 | (4-Iodo-phenyl)-[4-(4-methyl-2- methylamino-thiazol-5-yl)-pyrimidin-2-yl]- amine |
| 58 | 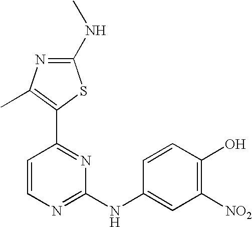 | 4-[4-(4-Methyl-2-methylamino-thiazol-5- yl)-pyrimidin-2-ylamino]-2-nitro-phenol |
| 59 | 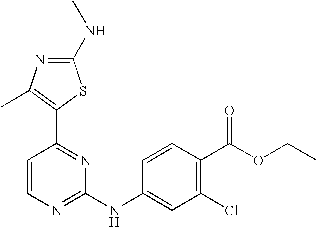 | 2-Chloro-4-[4-(4-methyl-2-methylamino- thiazol-5-yl)-pyrimidin-2-ylamino]-benzoic acid ethyl ester |
| 60 | 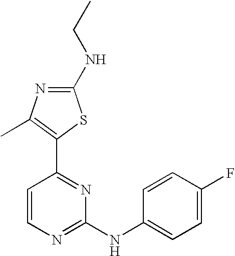 | [4-(2-Ethylamino-4-methyl-thiazol-5-yl)- pyrimidin-2-yl]-(4-fluoro-phenyl)-amine |
| 61 | 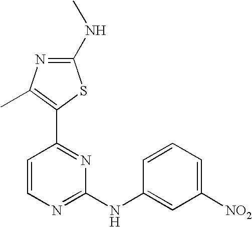 | [4-(4-Methyl-2-methylamino-thiazol-5-yl)- pyrimidin-2-yl]-(3-nitro-phenyl)-amine |
| 62 | 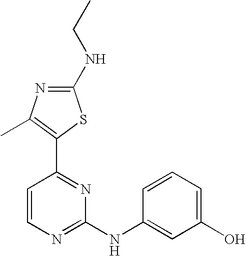 | 3-[4-(2-Ethylamino-4-methyl-thiazol-5-yl)- pyrimidin-2-ylamino]-phenol |
| 63 | 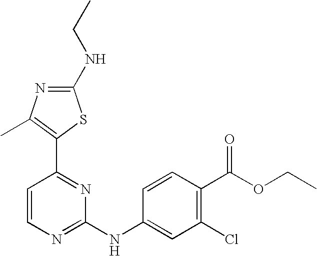 | 2-Chloro-4-[4-(2-ethylamino-4-methyl- thiazol-5-yl)-pyrimidin-2-ylamino]-benzoic acid ethyl ester |
| 64 | 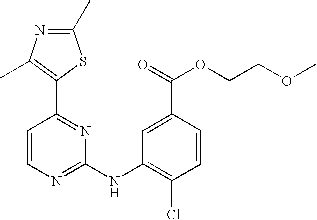 | 4-Chloro-3-[4-(2,4-dimethyl-thiazol-5-yl)- pyrimidin-2-ylamino]-benzoic acid 2- methoxy-ethyl ester |
| 65 | 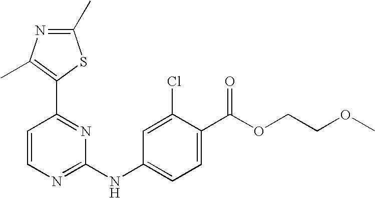 | 2-Chloro-4-[4-(2,4-dimethyl-thiazol-5-yl)- pyrimidin-2-ylamino]-benzoic acid 2- methoxy-ethyl ester |
| 66 | 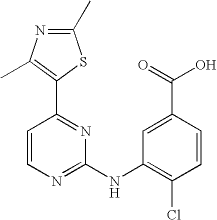 | 4-Chloro-3-[4-(2,4-dimethyl-thiazol-5-yl)- pyrimidin-2-ylamino]-benzoic acid |
| 67 | 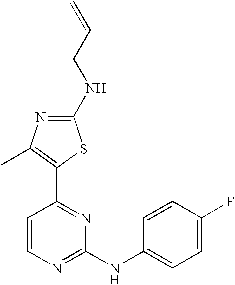 | [4-(2-Allylamino-4-methyl-thiazol-5-yl)- pyrimidin-2-yl]-(4-fluoro-phenyl)-amine |
| 68 | 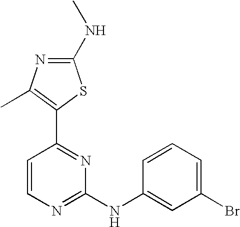 | (3-Bromo-phenyl)-[4-(4-methyl-2- methylamino-thiazol-5-yl)-pyrimidin-2-yl]- amine |
| 69 | 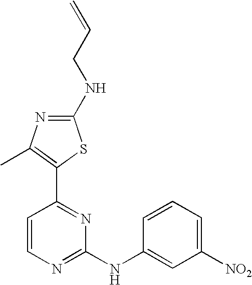 | [4-(2-Allylamino-4-methyl-thiazol-5-yl)- pyrimidin-2-yl]-(3-nitro-phenyl)-amine |
| 70 | 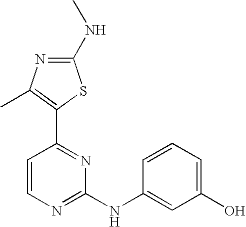 | 3-[4-(4-Methyl-2-methylamino-thiazol-5- yl)-pyrimidin-2-ylamino]-phenol |
| 71 | 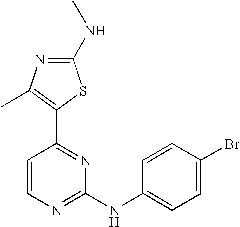 | (4-Bromo-phenyl)-[4-(4-methyl-2- methylamino-thiazol-5-yl)-pyrimidin-2-yl]- amine |
| 72 | 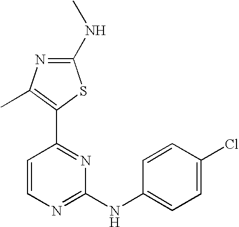 | (4-Chloro-phenyl)-[4-(4-methyl-2- methylamino-thiazol-5-yl)-pyrimidin-2-yl]- amine |
| 73 | 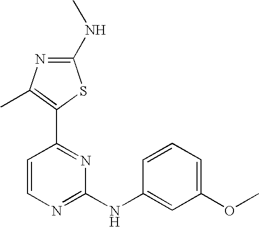 | (3-Methoxy-phenyl)-[4-(4-methyl-2- methylamino-thiazol-5-yl)-pyrimidin-2-yl]- amine |
| 74 | 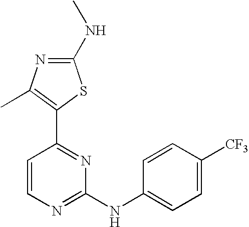 | [4-(4-Methyl-2-methylamino-thiazol-5-yl)- pyrimidin-2-yl]-(4-trifluoromethyl-phenyl)- amine |
| 75 | 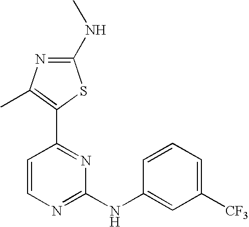 | [4-(4-Methyl-2-methylamino-thiazol-5-yl)- pyrimidin-2-yl]-(3-trifluoromethyl-phenyl)- amine |
| 76 | 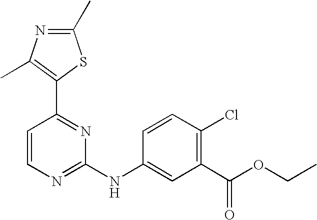 | 2-Chloro-5-[4-(2,4-dimethyl-thiazol-5-yl)- pyrimidin-2-ylamino]-benzoic acid ethyl ester |
| 77 | 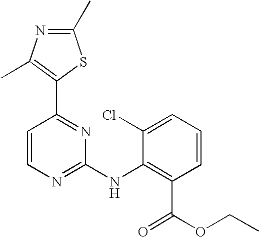 | 3-Chloro-2-[4-(2,4-dimethyl-thiazol-5-yl)- pyrimidin-2-ylamino]-benzoic acid ethyl ester |
| 78 | 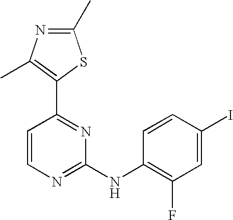 | [4-(2,4-Dimethyl-thiazol-5-yl)-pyrimidin-2- yl]-(2-fluoro-4-iodo-phenyl)-amine |
| 79 | 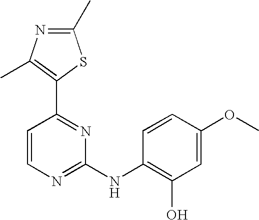 | 2-[4-(2,4-Dimethyl-thiazol-5-yl)-pyrimidin- 2-ylamino]-5-methoxy-phenol |
| 80 | 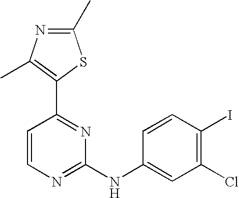 | (3-Chloro-4-iodo-phenyl)-[4-(2,4-dimethyl- thiazol-5-yl)-pyrimidin-2-yl]-amine |
| 81 | 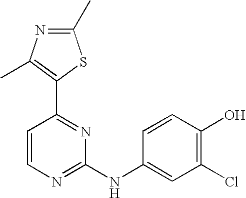 | 2-Chloro-4-[4-(2,4-dimethyl-thiazol-5-yl)- pyrimidin-2-ylamino]-phenol |
| 82 | 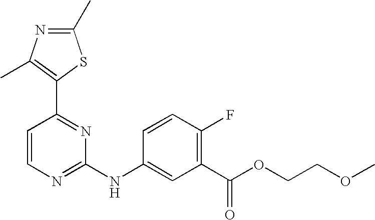 | 5-[4-(2,4-Dimethyl-thiazol-5-yl)-pyrimidin- 2-ylamino]-2-fluoro-benzoic acid 2- methoxy-ethyl ester |
| 83 | 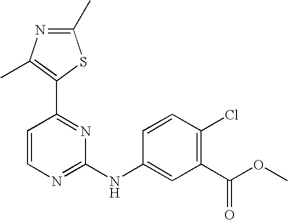 | 2-Chloro-5-[4-(2,4-dimethyl-thiazol-5-yl)- pyrimidin-2-ylamino]-benzoic acid methyl ester |
| 84 | 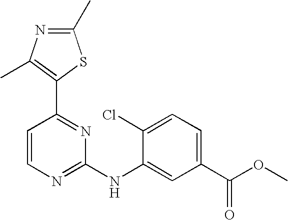 | 4-Chloro-3-[4-(2,4-dimethyl-thiazol-5-yl)- pyrimidin-2-ylamino]-benzoic acid methyl ester |
| 85 | 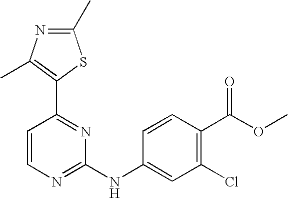 | 2-Chloro-4-[4-(2,4-dimethyl-thiazol-5-yl)- pyrimidin-2-ylamino]-benzoic acid methyl ester |
| 86 | 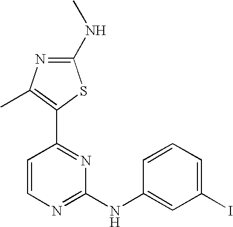 | (3-Iodo-phenyl)-[4-(4-methyl-2- methylamino-thiazol-5-yl)-pyrimidin-2-yl]- amine |
| 87 | 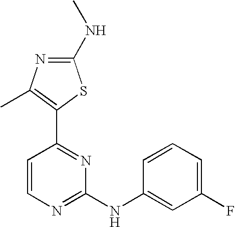 | (3-Fluoro-phenyl)-[4-(4-methyl-2- methylamino-thiazol-5-yl)-pyrimidin-2-yl]- amine |
| 88 | 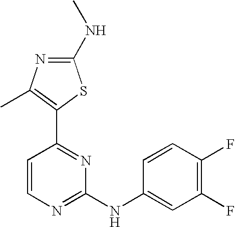 | (3,4-Difluoro-phenyl)-[4-(4-methyl-2- methylamino-thiazol-5-yl)-pyrimidin-2-yl]- amine |
| 89 | 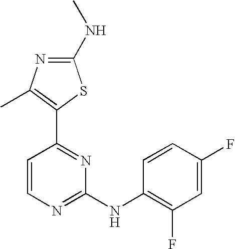 | (2,4-Difluoro-phenyl)-[4-(4-methyl-2- methylamino-thiazol-5-yl)-pyrimidin-2-yl]- amine |
| 90 | 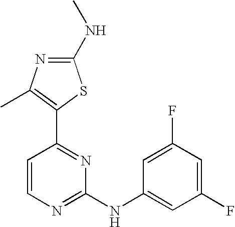 | (3,5-Difluoro-phenyl)-[4-(4-methyl-2- methylamino-thiazol-5-yl)-pyrimidin-2-yl]- amine |
| 91 | 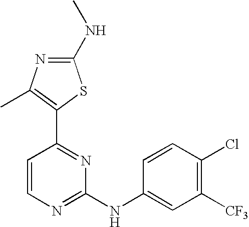 | (4-Chloro-3-trifluoromethyl-phenyl)-[4-(4- methyl-2-methylamino-thiazol-5-yl)- pyrimidin-2-yl]-amine |
| 92 | 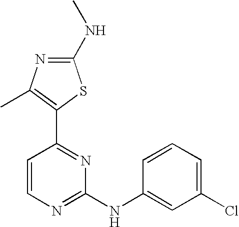 | (3-Chloro-phenyl)-[4-(4-methyl-2- methylamino-thiazol-5-yl)-pyrimidin-2-yl]- amine |
| 93 | 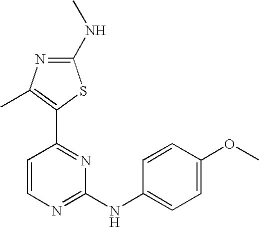 | (4-Methoxy-phenyl)-[4-(4-methyl-2- methylamino-thiazol-5-yl)-pyrimidin-2-yl]- amine |
| 94 | 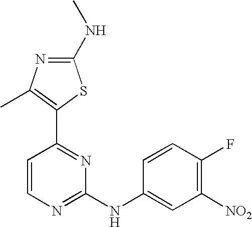 | (4-Fluoro-3-nitro-phenyl)-[4-(4-methyl-2- methylamino-thiazol-5-yl)-pyrimidin-2-yl]- amine |
| 95 | 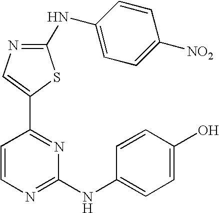 | 4-{4-[2-(4-Nitro-phenylamino)-thiazol-5- yl]-pyrimidin-2-ylamino}-phenol |
| 96 | 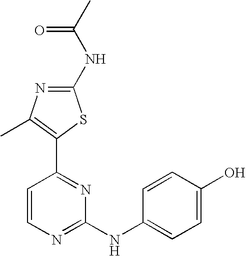 | N-{5-[2-(4-Hydroxy-phenylamino)- pyrimidin-4-yl]-4-methyl-thiazol-2-yl}- acetamide |
| 97 | 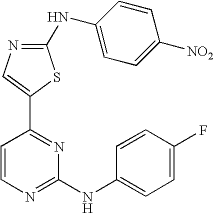 | (4-Fluoro-phenyl)-{4-[2-(4-nitro- phenylamino)-thiazol-5-yl]-pyrimidin-2- yl}-amine |
| 98 | 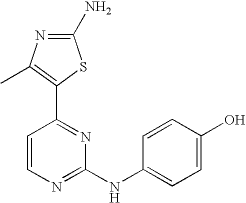 | 4-[4-(2-Amino-4-methyl-thiazol-5-yl)- pyrimidin-2-ylamino]-phenol |
| 99 | 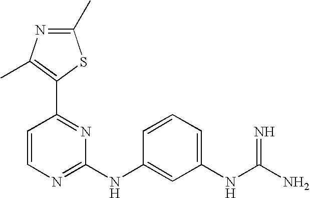 | N-{3-[4-(2,4-Dimethyl-thiazol-5-yl)- pyrimidin-2-ylamino]-phenyl}-guanidine |
| 100 | 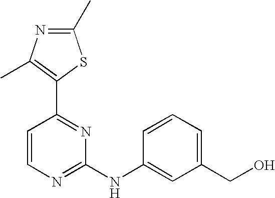 | {3-[4-(2,4-Dimethyl-thiazol-5-yl)- pyrimidin-2-ylamino]-phenyl}-methanol |
| 101 | 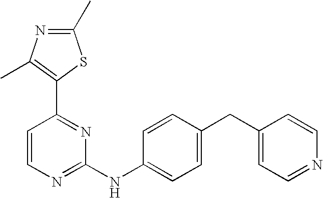 | [4-(2,4-Dimethyl-thiazol-5-yl)-pyrimidin-2- yl]-(4-pyridin-4-ylmethyl-phenyl)-amine |
| 102 | 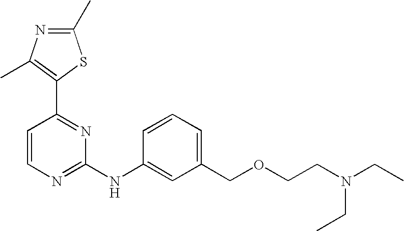 | [3-(2-Diethylamino-ethoxymethyl)- phenyl]-[4-(2,4-dimethyl-thiazol-5-yl)- pyrimidin-2-yl]-amine |
| 103 | 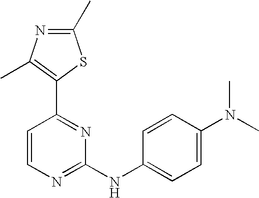 | N,N-Dimethyl-N′-[4-(2,4-dimethyl-thiazol- 5-yl)-pyrimidin-2-yl]-benzene-1,4-diamine |
| 104 | 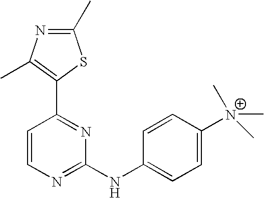 | {4-[4-(2,4-Dimethyl-thiazol-5-yl)- pyrimidin-2-ylamino]-phenyl}-trimethyl- ammonium |
| 105 | 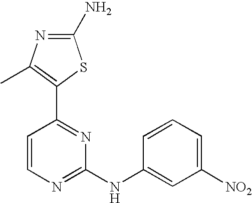 | [4-(2-Amino-4-methyl-thiazol-5-yl)- pyrimidin-2-yl]-(3-nitro-phenyl)-amine |
| 106 | 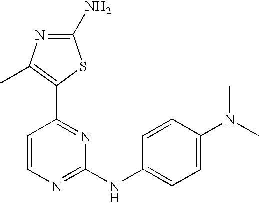 | N-[4-(2-Amino-4-methyl-thiazol-5-yl)- pyrimidin-2-yl]-N′,N′-dimethyl-benzene- 1,4-diamine |
| 107 | 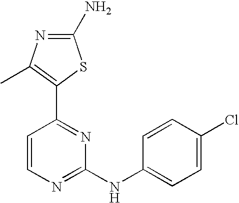 | [4-(2-Amino-4-methyl-thiazol-5-yl)- pyrimidin-2-yl]-(4-chloro-phenyl)-amine |
| 108 | 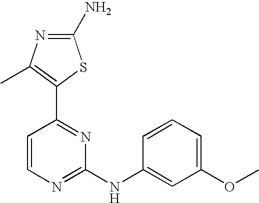 | [4-(2-Amino-4-methyl-thiazol-5-yl)- pyrimidin-2-yl]-(3-methoxy-phenyl)-amine |
| 109 | 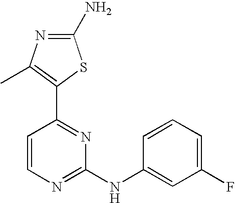 | [4-(2-Amino-4-methyl-thiazol-5-yl)- pyrimidin-2-yl]-(3-fluoro-phenyl)-amine |
| 110 | 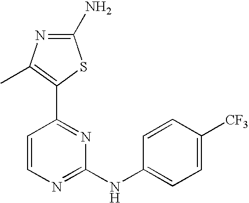 | [4-(2-Amino-4-methyl-thiazol-5-yl)- pyrimidin-2-yl]-(4-trifluoromethyl-phenyl)- amine |
| 111 | 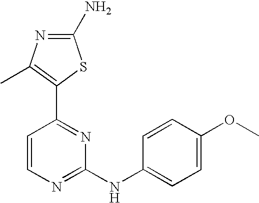 | [4-(2-Amino-4-methyl-thiazol-5-yl)- pyrimidin-2-yl]-(4-methoxy-phenyl)-amine |
| 112 | 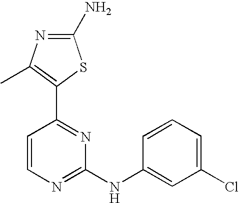 | [4-(2-Amino-4-methyl-thiazol-5-yl)- pyrimidin-2-yl]-(3-chloro-phenyl)-amine |
| 113 | 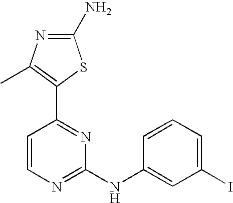 | [4-(2-Amino-4-methyl-thiazol-5-yl)- pyrimidin-2-yl]-(3-iodo-phenyl)-amine |
| 114 | 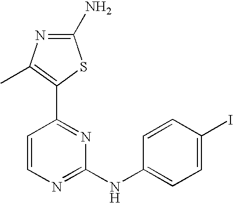 | [4-(2-Amino-4-methyl-thiazol-5-yl)- pyrimidin-2-yl]-(4-iodo-phenyl)-amine |
| 115 | 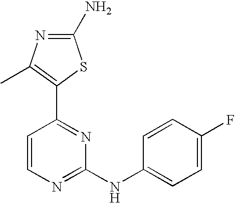 | [4-(2-Amino-4-methyl-thiazol-5-yl)- pyrimidin-2-yl]-(4-fluoro-phenyl)-amine |
| 116 | 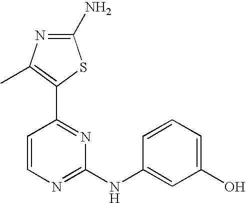 | 3-[4-(2-Amino-4-methyl-thiazol-5-yl)- pyrimidin-2-ylamino]-phenol |
| 117 | 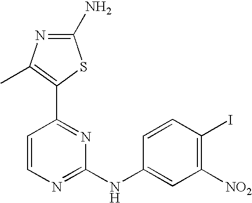 | [4-(2-Amino-4-methyl-thiazol-5-yl)- pyrimidin-2-yl]-(4-iodo-3-nitro-phenyl)- amine |
| 118 | 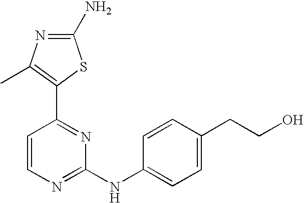 | 2-{4-[4-(2-Amino-4-methyl-thiazol-5-yl)- pyrimidin-2-ylamino]-phenyl}-ethanol |
| 119 | 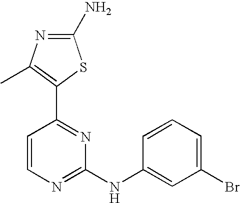 | [4-(2-Amino-4-methyl-thiazol-5-yl)- pyrimidin-2-yl]-(3-bromo-phenyl)-amine |
| 120 | 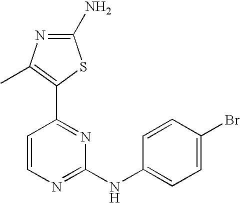 | [4-(2-Amino-4-methyl-thiazol-5-yl)- pyrimidin-2-yl]-(4-bromo-phenyl)-amine |
| 121 | 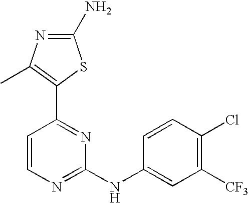 | [4-(2-Amino-4-methyl-thiazol-5-yl)- pyrimidin-2-yl]-(4-chloro-3- trifluoromethyl-phenyl)-amine |
| 122 | 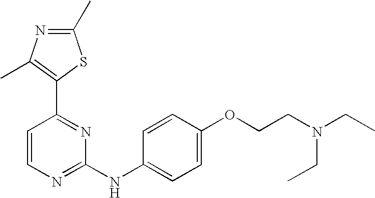 | [4-(2-Diethylamino-ethoxy)-phenyl]-[4- (2,4-dimethyl-thiazol-5-yl)-pyrimidin-2-yl]- amine |
| 123 | 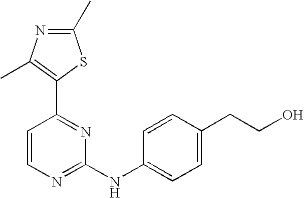 | 2-{4-[4-(2,4-Dimethyl-thiazol-5-yl)- pyrimidin-2-ylamino]-phenyl}-ethanol |
| 124 | 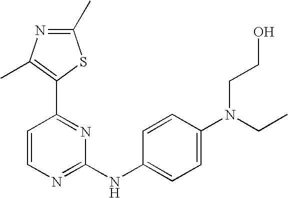 | 2-({4-[4-(2,4-Dimethyl-thiazol-5-yl)- pyrimidin-2-ylamino]-phenyl}-ethyl- amino)-ethanol |
| 125 | 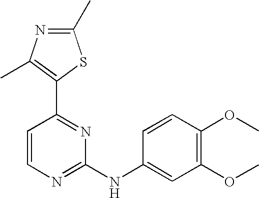 | (3,4-Dimethoxy-phenyl)-[4-(2,4-dimethyl- thiazol-5-yl)-pyrimidin-2-yl]-amine |
| 126 | 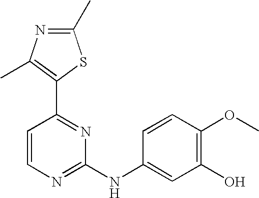 | 5-[4-(2,4-Dimethyl-thiazol-5-yl)-pyrimidin- 2-ylamino]-2-methoxy-phenol |
| 127 | 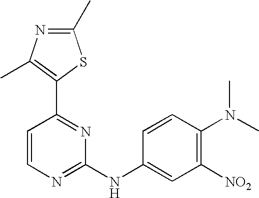 | N⁴-[4-(2,4-Dimethyl-thiazol-5-yl)- pyrimidin-2-yl]-N¹,N¹-dimethyl-2-nitro- benzene-1,4-diamine |
| 128 | 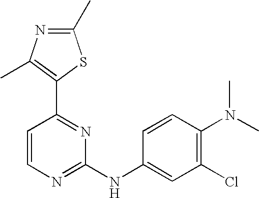 | 2-Chloro-N⁴-[4-(2,4-dimethyl-thiazol-5-yl)- pyrimidin-2-yl]-N¹,N¹-dimethyl-benzene- 1,4-diamine |
| 129 | 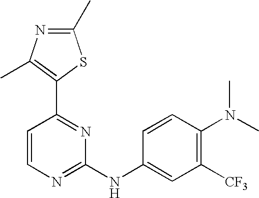 | N⁴-[4-(2,4-Dimethyl-thiazol-5-yl)- pyrimidin-2-yl]-N¹,N¹-dimethyl-2- trifluoromethyl-benzene-1,4-diamine |
| 130 | 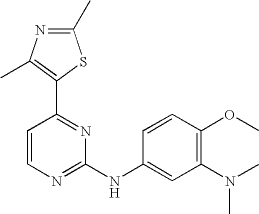 | N¹-[4-(2,4-Dimethyl-thiazol-5-yl)- pyrimidin-2-yl]-4-methoxy-N³,N³- dimethyl-benzene-1,3-diamine |
| 131 | 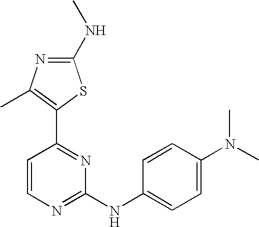 | N,N-Dimethyl-N′-[4-(4-methyl-2- methylamino-thiazol-5-yl)-pyrimidin-2-yl]- benzene-1,4-diamine |
| 132 | 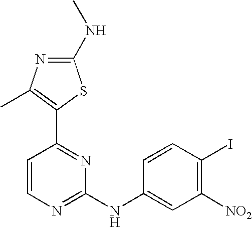 | (4-Iodo-3-nitro-phenyl)-[4-(4-methyl-2- methylamino-thiazol-5-yl)-pyrimidin-2-yl]- amine |
| 133 | 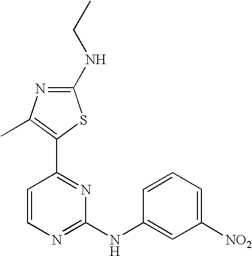 | [4-(2-Ethylamino-4-methyl-thiazol-5-yl)- pyrimidin-2-yl]-(3-nitro-phenyl)-amine; |
| 134 | 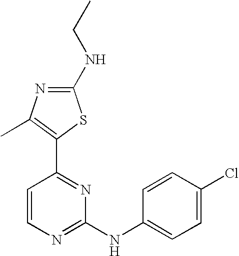 | (4-Chloro-phenyl)-[4-(2-ethylamino-4- methyl-thiazol-5-yl)-pyrimidin-2-yl]- amine; |
| 135 | 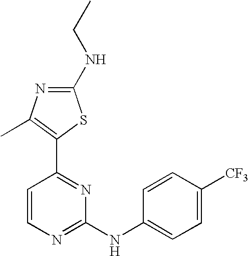 | [4-(2-Ethylamino-4-methyl-thiazol-5-yl)- pyrimidin-2-yl]-(4-trifluoromethyl-phenyl)- amine |
| 136 | 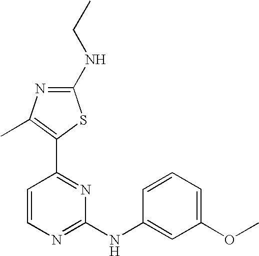 | [4-(2-Ethylamino-4-methyl-thiazol-5-yl)- pyrimidin-2-yl]-(3-methoxy-phenyl)-amine |
| 137 | 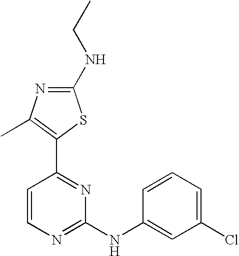 | (3-Chloro-phenyl)-[4-(2-ethylamino-4- methyl-thiazol-5-yl)-pyrimidin-2-yl]-amine |
| 138 | 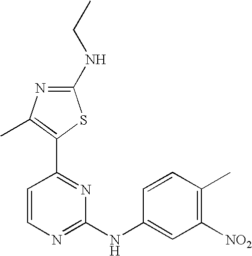 | [4-(2-Ethylamino-4-methyl-thiazol-5-yl)- pyrimidin-2-yl]-(4-methyl-3-nitro-phenyl)- amine |
| 139 | 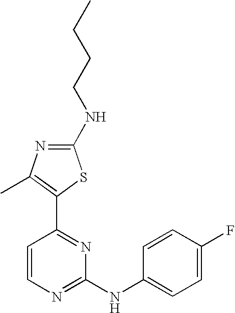 | [4-(2-Butylamino-4-methyl-thiazol-5-yl)- pyrimidin-2-yl]-(4-fluoro-phenyl)-amine |
| 140 | 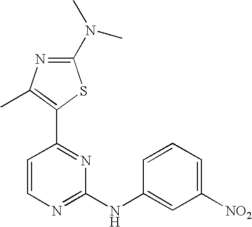 | [4-(2-Dimethylamino-4-methyl-thiazol-5- yl)-pyrimidin-2-yl]-(3-nitro-phenyl)-amine |
| 141 | 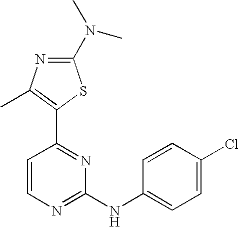 | (4-Chloro-phenyl)-[4-(2-dimethylamino-4- methyl-thiazol-5-yl)-pyrimidin-2-yl]-amine |
| 142 | 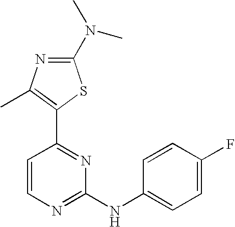 | [4-(2-Dimethylamino-4-methyl-thiazol-5- yl)-pyrimidin-2-yl]-(4-fluoro-phenyl)- amine |
| 143 | 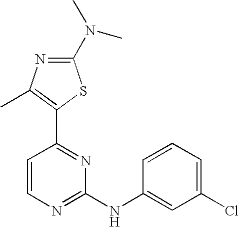 | (3-Chloro-phenyl)-[4-(2-dimethylamino-4- methyl-thiazol-5-yl)-pyrimidin-2-yl]-amine |
| 144 | 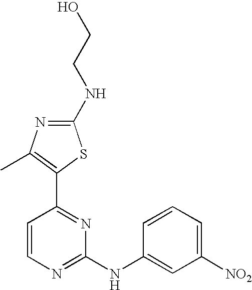 | 2-{4-Methyl-5-[2-(3-nitro-phenylamino)- pyrimidin-4-yl]-thiazol-2-ylamino}-ethanol |
| 145 | 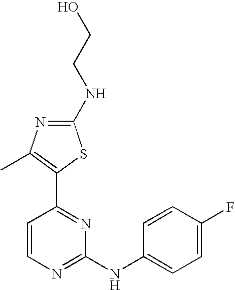 | 2-{5-[2-(4-Fluoro-phenylamino)-pyrimidin- 4-yl]-4-methyl-thiazol-2-ylamino}-ethanol |
| 146 | 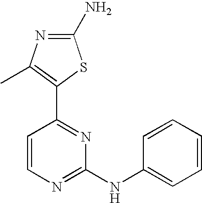 | [4-(2-Amino-4-methyl-thiazol-5-yl)- pyrimidin-2-yl]-phenyl-amine |
| 147 | 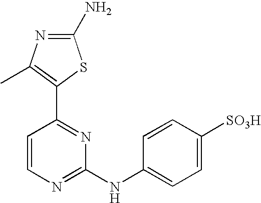 | 4-[4-(2-Amino-4-methyl-thiazol-5-yl)- pyrimidin-2-ylamino]-benzenesulfonic acid |
| 148 | 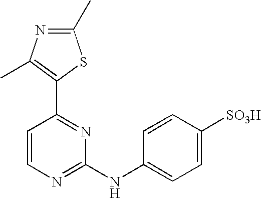 | 4-[4-(2,4-Dimethyl-thiazol-5-yl)-pyrimidin- 2-ylamino]-benzenesulfonic acid |
| 149 | 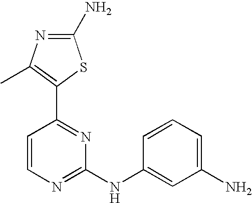 | N-[4-(2-Amino-4-methyl-thiazol-5-yl)- pyrimidin-2-yl]-benzene-1,3-diamine |
| 150 | 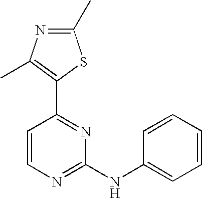 | [4-(2,4-Dimethyl-thiazol-5-yl)-pyrimidin-2- yl]-phenyl-amine |
| 151 | 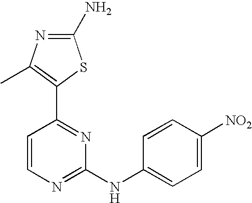 | [4-(2-Amino-4-methyl-thiazol-5-yl)- pyrimidin-2-yl]-(4-nitro-phenyl)-amine |
| 152 | 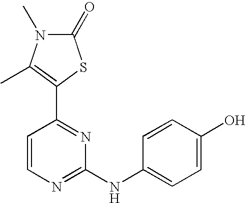 | 5-[2-(4-Hydroxy-phenylamino)-pyrimidin- 4-yl]-3,4-dimethyl-3H-thiazol-2-one |
| 153 | 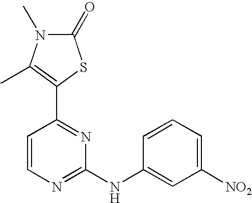 | 3,4-Dimethyl-5-[2-(3-nitro-phenylamino)- pyrimidin-4-yl]-3H-thiazol-2-one |
| 154 | 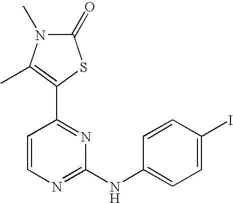 | 5-[2-(4-Iodo-phenylamino)-pyrimidin-4- yl]-3,4-dimethyl-3H-thiazol-2-one |
| 155 | 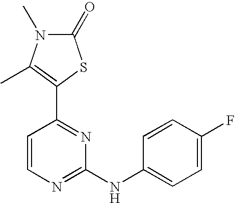 | 5-[2-(4-Fluoro-phenylamino)-pyrimidin-4- yl]-3,4-dimethyl-3H-thiazol-2-one |
| 156 | 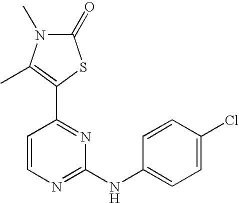 | 5-[2-(4-Chloro-phenylamino)-pyrimidin-4- yl]-3,4-dimethyl-3H-thiazol-2-one |
| 157 | 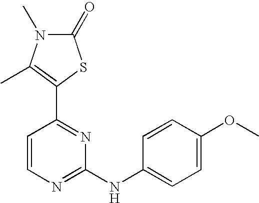 | 5-[2-(4-Methoxy-phenylamino)-pyrimidin- 4-yl]-3,4-dimethyl-3H-thiazol-2-one |
| 158 | 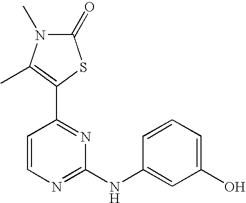 | 5-[2-(3-Hydroxy-phenylamino)-pyrimidin- 4-yl]-3,4-dimethyl-3H-thiazol-2-one |
| 159 | 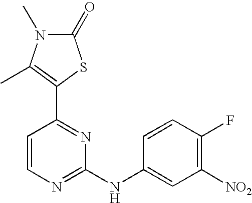 | 5-[2-(4-Fluoro-3-nitro-phenylamino)- pyrimidin-4-yl]-3,4-dimethyl-3H-thiazol-2- one |
| 160 | 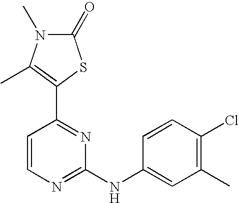 | 5-[2-(4-Chloro-3-methyl-phenylamino)- pyrimidin-4-yl]-3,4-dimethyl-3H-thiazol-2- one |
| 161 | 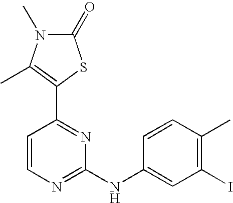 | 5-[2-(3-Iodo-4-methyl-phenylamino)- pyrimidin-4-yl]-3,4-dimethyl-3H-thiazol-2- one |
| 162 | 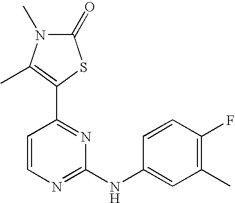 | 5-[2-(4-Fluoro-3-methyl-phenylamino)- pyrimidin-4-yl]-3,4-dimethyl-3H-thiazol-2- one |
| 163 | 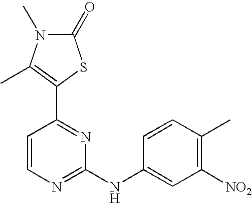 | 3,4-Dimethyl-5-[2-(4-methyl-3-nitro- phenylamino)-pyrimidin-4-yl]-3H-thiazol- 2-one |
| 164 | 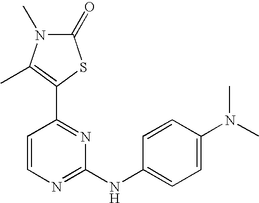 | 5-[2-(4-Dimethylamino-phenylamino)- pyrimidin-4-yl]-3,4-dimethyl-3H-thiazol-2- one |

TABLE 2

| | Average IC₅₀ (μM) | | | | | | | | | | | | |
|---|---|---|---|---|---|---|---|---|---|---|---|---|---|
| | CDK1 | CDK2 | CDK4 | CDK7 | CDK9 | | | | | | | | |
| Cmpd | cyclin | cyclin | cyclin | cyclin | cyclin | Akt/ | | | | | | | |
| No. | B1 | E1 | D1 | H | T1 | PKB | CaMKII | CK2 | ERK2 | PKA | PKC | S6 | SAPK2a |
| 5 | 17 | 0.23 | >50 | 13 | 0.17 | >50 | >50 | >50 | 40 | >50 | 35 | 44 | 40 |
| 9 | 38 | 6.3 | >50 | | | >50 | >50 | >50 | 55 | 39 | 40 | 30 | 40 |
| 15 | 34 | 0.94 | >50 | >100 | | >50 | >50 | >50 | >50 | >50 | >50 | >50 | >50 |
| 26 | 9.0 | 0.22 | 2.4 | | | >50 | >50 | >50 | >50 | >50 | >50 | 31 | 45 |
| 36 | 2.5 | 0.39 | 2.9 | 1.7 | 1.0 | >50 | >50 | >50 | >50 | 55 | >50 | >50 | >50 |
| 105 | 1.3 | 0.028 | 0.086 | 0.11 | 0.012 | >100 | >100 | >100 | >100 | 65 | >100 | 59 | >100 |
| 116 | 0.58 | 0.11 | 0.46 | 0.99 | 0.13 | 53 | 7.7 | 2.8 | 15 | 3.7 | 33 | 4.1 | 29 |
| 117 | 1.7 | 0.28 | 1.4 | 0.36 | 0.055 | 70 | >100 | >100 | >100 | >50 | >100 | 11 | >100 |

TABLE 3

| | Average IC₅₀ (μM) | | | | | |
|---|---|---|---|---|---|---|
| Compound | CDK1 | CDK2 | CDK2 | CDK4 | CDK7 | CDK9 |
| No. | cyclin B1 | cyclin A2 | cyclin E1 | cyclin D1 | cyclin H | cyclin T1 |
| 2 | 14 | n.d. | 5.1 | n.d. | n.d. | n.d. |
| 3 | 32 | n.d. | 5.6 | >50 | >100 | n.d. |
| 5 | 17 | n.d. | 0.23 | >50 | 13 | 0.17 |
| 6 | n.d. | n.d. | 8.0 | n.d. | n.d. | n.d. |
| 7 | 12 | n.d. | 2.5 | n.d. | n.d. | n.d. |
| 8 | 17 | n.d. | 0.071 | >50 | 16 | 0.31 |
| 9 | 38 | n.d. | 6.3 | >50 | n.d. | n.d. |
| 10 | 5.5 | n.d. | 5.9 | n.d. | n.d. | n.d. |
| 11 | 20 | n.d. | 10 | n.d. | n.d. | n.d. |
| 12 | n.d. | n.d. | 10 | n.d. | n.d. | n.d. |
| 13 | >100 | >100 | 22 | >100 | >100 | 85 |
| 15 | 34 | n.d. | 0.94 | >50 | >100 | n.d. |
| 17 | n.d. | n.d. | 0.94 | n.d. | n.d. | n.d. |
| 20 | n.d. | n.d. | 6.8 | n.d. | n.d. | n.d. |
| 22 | n.d. | n.d. | 0.22 | n.d. | n.d. | n.d. |
| 23 | n.d. | n.d. | 0.50 | n.d. | n.d. | 3.113 |
| 24 | n.d. | n.d. | 6.3 | n.d. | n.d. | n.d. |
| 25 | n.d. | n.d. | 0.44 | n.d. | n.d. | n.d. |
| 26 | 9.0 | n.d. | 0.22 | 2.4 | n.d. | n.d. |
| 27 | 1.2 | n.d. | 0.11 | 0.43 | 3.2 | 0.24 |
| 28 | 1.8 | n.d. | 0.28 | 0.60 | 2.4 | 0.46 |
| 32 | 14 | n.d. | 0.21 | 0.38 | n.d. | 0.055 |
| 34 | 1.5 | 0.53 | 0.53 | 1.7 | 5.2 | 0.41 |
| 35 | >50 | n.d. | 8.0 | >50 | n.d. | n.d. |
| 36 | 2.5 | 0.16 | 0.39 | 2.9 | 1.7 | 1.0 |
| 37 | 38 | n.d. | 7.5 | 17 | n.d. | n.d. |
| 39 | 40 | n.d. | 5.6 | >50 | n.d. | n.d. |
| 40 | n.d. | n.d. | 0.74 | n.d. | n.d. | n.d. |
| 41 | n.d. | n.d. | 0.58 | n.d. | n.d. | n.d. |
| 47 | n.d. | n.d. | 0.037 | n.d. | n.d. | 0.082 |
| 48 | n.d. | n.d. | 0.006 | n.d. | 0.86 | 0.076 |
| 58 | n.d. | n.d. | 1.5 | n.d. | n.d. | 0.061 |
| 60 | n.d. | n.d. | 0.51 | n.d. | n.d. | n.d. |
| 61 | n.d. | n.d. | 2.1 | n.d. | n.d. | 0.19 |
| 67 | n.d. | n.d. | 1.3 | n.d. | n.d. | n.d. |
| 68 | n.d. | n.d. | 27 | n.d. | n.d. | 0.010 |
| 69 | n.d. | n.d. | 0.32 | n.d. | n.d. | 0.068 |
| 70 | n.d. | n.d. | 0.27 | n.d. | 3.4 | 0.048 |
| 73 | n.d. | n.d. | 0.31 | n.d. | n.d. | 0.48 |
| 74 | n.d. | n.d. | 1.9 | n.d. | n.d. | n.d. |
| 75 | >100 | >100 | 0.77 | >100 | 19 | 8.3 |
| 79 | n.d. | n.d. | 1.2 | n.d. | n.d. | n.d. |
| 83 | n.d. | n.d. | 2.0 | n.d. | n.d. | n.d. |
| 87 | n.d. | n.d. | 1.3 | n.d. | n.d. | 0.063 |
| 93 | n.d. | n.d. | 0.001 | n.d. | n.d. | n.d. |
| 95 | n.d. | n.d. | 0.12 | n.d. | n.d. | n.d. |
| 98 | n.d. | n.d. | n.d. | n.d. | 1.0 | 0.045 |
| 99 | n.d. | n.d. | n.d. | n.d. | 0.31 | 0.26 |
| 100 | n.d. | n.d. | 0.19 | n.d. | n.d. | n.d. |
| 101 | n.d. | n.d. | 0.69 | 1.1 | n.d. | n.d. |
| 103 | 7.3 | 1.1 | 0.51 | 2.9 | 11 | 1.8 |
| 104 | n.d. | n.d. | n.d. | n.d. | 0.39 | 0.14 |
| 105 | 1.3 | 0.33 | 0.028 | 0.086 | 0.11 | 0.012 |
| 106 | n.d. | n.d. | n.d. | n.d. | n.d. | 0.24 |
| 108 | 2.8 | 0.91 | 0.39 | 1.5 | n.d. | n.d. |
| 109 | 2.9 | 1.4 | 0.38 | 1.2 | n.d. | n.d. |
| 110 | n.d. | n.d. | 0.68 | 5.8 | n.d. | n.d. |
| 111 | n.d. | n.d. | 0.67 | n.d. | n.d. | n.d. |
| 112 | n.d. | n.d. | 0.018 | 0.071 | n.d. | n.d. |
| 113 | n.d. | n.d. | n.d. | n.d. | 0.59 | n.d. |
| 116 | 0.58 | 0.18 | 0.11 | 0.46 | 0.99 | 0.13 |
| 117 | 1.7 | 1.7 | 0.28 | 1.4 | 0.36 | 0.055 |
| 118 | 1.8 | 0.42 | 0.39 | 0.82 | 2.8 | 0.80 |
| 119 | n.d. | n.d. | n.d. | n.d. | n.d. | 0.28 |
| 120 | n.d. | n.d. | 1.0 | 1.6 | n.d. | n.d. |
| 122 | n.d. | n.d. | n.d. | n.d. | 0.44 | n.d. |
| 123 | 1.9 | 0.57 | 0.47 | 3.3 | n.d. | n.d. |
| 124 | 8.6 | 3.7 | 3.0 | 6.7 | n.d. | n.d. |
| 125 | 0.25 | 0.26 | 0.033 | 1.1 | 5.9 | 0.59 |
| 126 | 1.6 | 0.31 | 0.14 | 1.7 | n.d. | 1.3 |
| 127 | 0.13 | 0.071 | 0.037 | 0.68 | 1.5 | 0.097 |
| 128 | 2.3 | 1.7 | 0.60 | 1.9 | 6.3 | 0.45 |
| 130 | 0.47 | 0.67 | 1.1 | 4.1 | 4.9 | n.d. |
| 131 | n.d. | n.d. | n.d. | n.d. | n.d. | 0.17 |
| 132 | n.d. | n.d. | n.d. | n.d. | n.d. | 0.11 |
| 133 | >100 | 4.2 | 0.088 | 4.7 | 0.39 | 0.21 |
| 134 | 22 | n.d. | 2.0 | n.d. | n.d. | 5.9 |
| 136 | >100 | 39 | 6.8 | n.d. | n.d. | n.d. |
| 138 | 41 | 9.5 | 2.7 | n.d. | n.d. | n.d. |
| 139 | 47 | 25 | 1.7 | 30 | n.d. | 23 |
| 140 | 49 | 1.8 | 0.48 | 105 | >100 | 7.1 |
| 141 | n.d. | n.d. | 3.9 | 4.8 | n.d. | n.d. |
| 142 | n.d. | n.d. | 1.3 | 4.0 | n.d. | n.d. |
| 143 | n.d. | n.d. | 0.89 | 5.4 | n.d. | n.d. |
| 144 | n.d. | n.d. | n.d. | n.d. | n.d. | 0.004 |
| 145 | n.d. | n.d. | n.d. | n.d. | n.d. | 0.008 |
| 148 | 0.048 | 0.001 | 0.028 | 3.8 | 11 | n.d. |
| 149 | 0.71 | 0.52 | 0.25 | 0.50 | 0.99 | 0.059 |
| 150 | 1.5 | 0.41 | 0.16 | 4.9 | 11 | n.d. |
| 151 | n.d. | n.d. | 0.18 | >50 | n.d. | n.d. |

n.d.: not determined

TABLE 4
Inhibition of protein kinases by example compounds

| | Kinase Inhibition IC₅₀ (μM) | | | | | |
|---|---|---|---|---|---|---|
| | CDK1- | CDK2- | CDK2- | CDK4- | CDK7- | CDK9- |
| No. | cyclin B | cyclin E | cyclin A | cyclin D1 | cyclin H | cyclin T1 |
| 153 | 3.0 | 0.61 | 0.016 | 0.56 | 0.12 | 0.0022 |
| 155 | | | | | | 0.0027 |
| 152 | | | | | | 0.0023 |
| 156 | 1.4 | 0.74 | 0.27 | 2.9 | 8.7 | |
| 157 | 0.78 | 0.70 | 0.76 | 1.1 | 2.0 | |
| 158 | 0.37 | 0.093 | 0.086 | 1.0 | 1.3 | |
| 164 | 2.2 | 0.75 | 1.5 | 1.0 | 1.8 | 0.13 |
| 159 | 0.82 | 0.078 | 0.26 | 1.8 | 1.1 | |
| 163 | 0.096 | 36 | 0.084 | 0.11 | 0.0012 | 0.014 |
| 162 | 1.1 | | 0.13 | | | 0.079 |
| 161 | 2.1 | 3.2 | 0.71 | 4.0 | | 0.11 |
| 160 | 1.8 | 1.7 | 1.2 | 18 | | 0.15 |
| 154 | | | | | | |

TABLE 5
Summary of anti-HIV activity

| | HIV-1_{RoJo}/PBMC | | | | HIV-1_{WeJo}/PBMC | | | |
|---|---|---|---|---|---|---|---|---|
| | IC₅₀ | IC₉₀ | TC₅₀ | | IC₅₀ | IC₉₀ | TC₅₀ | |
| Compound | (μM) | (μM) | (μM) | TI | (μM) | (μM) | (μM) | TI |
| AZT^{a} | 0.004 | 0.010 | >1.0 | >231 | 0.007 | 0.043 | >1.0 | >138 |
| 21 | 0.062 | 0.10 | 31 | 495 | 0.029 | 0.048 | 64 | 2190 |
| 28 | 0.28 | 21 | 22 | 80 | | | | |
| 47 | 0.20 | 1.2 | 6.8 | 34 | | | | |
| 70 | 0.63 | 2.6 | 2.7 | 4.3 | | | | |
| 103 | 0.26 | 0.32 | >100. | >380 | 0.037 | 0.48 | 0.8 | 23 |
| 105 | 0.067 | 0.35 | 1.6 | 24 | 0.005 | 0.014 | 0.2 | 46 |
| 125 | 0.82 | 0.97 | 27 | 33 | | | | |
| 128 | 0.74 | 2.7 | 4.3 | 5.9 | 0.86 | 2.0 | >100 | >117 |

^{a}AZT: Azidothymidine; anti-HIV drug in clinical use as positive control.

1. A method of treating a viral disorder in a subject, comprising administering to the subject one or more compounds of formula I 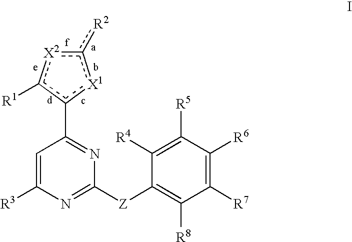
wherein:
(A) one of X¹ and X² is S, and the other of X¹ and X² is N;
“a” is a single bond; and
“b”, “c”, “d”, “e” and “f” are single or double bonds so as to form a thiazolyl ring;
R² is independently as defined below for R¹ and R³; or
(B) one of X¹ and X² is S, and the other of X¹ and X² is NR⁹;
“a” and “d” are each double bonds; and
“b”, “c”, “e” and “f” are each single bonds;
R is oxo;
R⁹ is H or alkyl;
where:
Z is NH, NHCO, NHSO₂, NHCH₂, CH₂, CH₂CH₂, or CH═CH;
R¹ and R³ are independently H, alkyl, aryl, aralkyl, heterocycle, halogeno, NO₂, CN, OH, alkoxy, aryloxy, NH₂, NH-alkyl, N—(R′)(R″), NH-aryl, N-(aryl)₂, NHCOR′, COOH, COO-alkyl, COO-aryl, CONH₂, CONH—R′, CON—(R′)(R″), CONH-aryl, CON-(aryl)₂, SO₃H, SO₂NH₂, CF₃, CO—R′, or CO-aryl, wherein said alkyl, NH-aryl, COO-alkyl, NH-alkyl, aryl, aralkyl and heterocycle groups may be further substituted with one or more groups selected from halogeno, NO₂, CN, OH, O-methyl, NH₂, COOH, N—(R′)(R″), CONH₂ and CF₃;
R⁴, R⁵, R⁶, R⁷, and R⁸ are independently from each other H, substituted or unsubstituted lower alkyl, halogeno, NO₂, CN, OH, substituted or unsubstituted alkoxy, NH₂, NH—R′, alkyl-aryl, alkyl-heteroaryl, NH(C═NH)NH₂, N(R′)₃⁺, N—(R′)(R″), COOH, COO—R′, CONH₂, CONH—R′, CON—(R′)(R″), SO₃H, SO₂NH₂, CF₃ or (CH₂)ₙO(CH₂)ₘNR′R″, (CH₂)ₙCO₂(CH₂)ₘOR′″ wherein n is 0, 1, 2 or 3 and m is 1, 2 or 3;
wherein R′ and R″ are each independently substituted or unsubstituted alkyl or alkenyl groups that may be the same or different; and pharmaceutically acceptable salts thereof, such that said subject is treated for said viral disorder.

2. The method according to claim 1, wherein said compound is of formula Ia 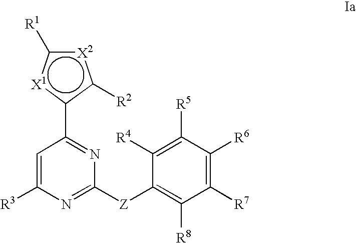
wherein one of X¹ and X² is S, and the other of X¹ and X² is N, and R¹⁻⁸ are as defined in claim 1.

3. The method of claim 1, wherein;
X¹ and X² are S and N respectively;
R¹, R² and R³ are each independently selected from H, alkyl, aryl, aralkyl, halogeno, NO₂, CN, OH, alkoxy, aryloxy, NH₂, NHCOR′, NHCOR′, NH-aryl, NH-alkyl, N—(R′)(R″), COOH, COO-alkyl, CONH₂, CONH—R′, CON—(R′)(R″), SO₃H, SO₂NH₂, CF₃, and CO—R′ wherein alkyl, aryl, COO-alkyl, NH-alkyl, NH-aryl and aralkyl groups may be further substituted with one or more groups selected from halogeno, NO₂, CN, OH, O-methyl, NH₂, COOH, CONH₂ and CF₃;
Z is selected from N, NHSO₂ and NHCH₂;
R⁴-R⁸ are each independently selected from H, OH, halogeno, nitro, amino, alkoxy, carbamoyl, sulfamyl, C₁₋₄ alkyl, substituted C₁₋₄ alkyl, SO₃H, COOH, COOR′, CN, CF₃, (CH₂)ₙO(CH₂)ₘNR′R″, alkyl-aryl, alkyl-heteroaryl, NH(C═NH)NH₂, N(R′)₃⁺, N(R′)(R″) and (CH₂)ₙCO₂(CH₂)ₘOR′″.

4. The method of claim 1, wherein X¹ and X² are S and N respectively.

5. The method of claim 1, wherein Z is NH.

6. The method of claim 1,. wherein R¹ and R² are each independently one or more of halogeno, a C₁₋₄ alkyl group, H, aryl, heterocycle, alkoxy, NH₂, NH-alkyl or N(R′)(R″).

7. The method of claim 1, wherein R³ is selected from H, aryl, substituted aryl, halo, C₁₋₄ alkoxy and OH.

8. The method of claim 1, wherein R³ is H.

9. The method of claim 1, wherein R⁴ to R⁸are selected independently from F, NH₂, NO₂, OH, Cl, Br, I, CF₃, OMe, COOH, COOR′, CN, H, C₁₋₄ alkyl, C₁₋₄ alkoxy, CH₂CO₂CH₂CH₂OMe, NH(C═NH)NH₂, CH₂CH₂OH, OCH₂CH₂NEt₂, SO₃H, N(Et)CH₂CH₂OH, CO₂CH₂CH₂OMe, CH₂OCH₂CH₂NEt₂, CH₂-heteroaryl, NMe₃⁺, and NMe₂.

10. The method of claim 1, wherein said compound is selected from the group consisting of:
(a) 2-[N-(phenyl)]-4-(2,4-dimethylthiazol-5-yl)pyrimidineamines in which the phenyl group is 2-, 3- or 4-substituted by at least one of Me, F, NH₂, NO₂, OH, Cl, Br, I, CF₃, OMe, CN, COOH, CH₂OH, COOMe, COOEt, NH(C═NH)NH₂, CH₂CO₂CH₂CH₂OMe, CH₂-pyridyl, CH₂OCH₂CH₂NEt₂, CH₂CH₂OH, N(Et)CH₂CH₂OH, OCH₂CH₂NEt₂, CO₂CH₂CH₂OMe, NMe₃⁺ and NMe₂;
(b) 2-[N-(phenyl)]-4-(2-amino-4-methylthiazol-5-yl)pyrimidineamines in which the phenyl group is 2-, 3- or 4-substituted by at least one of NO₂, NH₂, Cl, CH₂CH₂OH, OMe, F, CF₃, I, Br, SO₃H, N(R′)R″), OH, or NH₂;
(c) 2-[N-(phenyl)]-4-(2-methoxy-4-methylthiazol-5-yl)pyrimidineamines in which the phenyl group is 2-, 3- or 4-substituted by at least one of N(R′)R″), OH, OMe, NO₂, Me, I, Cl or F; and
(d) 2-[N-(phenyl)]-4-(4-methyl-2-methylamino-thiazol-5-yl)pyrimidineamines or 2-[N-(phenyl)]-4-(4-methyl-2-ethylamino-thiazol-5-yl)pyrimidineamines in which the phenyl group is 2-, 3- or 4-substituted by at least one of F, N(R′)R″), Me, OH, I, NO₂, Cl, COOR′, Br, OMe or CF₃.

11. The method of claim 10, wherein;
for group (a) the phenyl group is mono-substituted by OCH₂CH₂NEt₂, CH₂CH₂OH, N(Et)CH₂CH₂OH, SO₃H, NMe₂, F, NH₂, NO₂, OH, Cl, Br, I, CF₃, OMe, CN, CH₂OH, COOH, COOMe, COOEt, CH₂CO₂CH₂CH₂OMe or CO₂CH₂CH₂OMe at any of the 2,3 or 4-positions, or di-substituted by 2,4-difluoro, 3,5-difluoro, 3,4-difluoro, 2,4-dichloro, 3,5-dichloro, 3,4-dichloro, 4-hydroxy-2-nitro, 4-hydroxy-3-nitro, 6-chloro-3-carboxy, 4-chloro-3-carboxy, 6-chloro-2-carboxy, 2-fluoro-4-iodo, 2-hydroxy-4-methoxy, 3-chloro-4-iodo, 3-chloro-4-hydroxy, 3-chloro-4-methyl, 3-chloro-4-methoxy, 4-fluoro-3-nitro, 6-chloro-3-methoxycarbonyl, 3-chloro-4-methoxcarbonyl, 3-chloro-4-ethoxcarbonyl, 3,4-dimethoxy, 3-hydroxy-4-methoxy, 4-dimethylamino-3-nitro, 2-chloro-5-methoxycarbonyl, 4-chloro-3-methoxycarbonyl, 6-chloro-3-(CO₂CH₂CH₂OMe), 3-chloro-4-(CO₂CH₂CH₂OMe), 4-chloro-3-trifluoromethyl, 3-chloro-4-dimethylamino, 3-dimethylamino-4-methoxy or 3-(CO₂CH₂CH₂OMe)-4-fluoro;
for group (b) the phenyl group is mono-substituted by NH₂, SO₃H, N(R′)(R″), OMe, F, Cl, Br, I, CH₂CH₂OH, nitro or OH at any of the 2,3 or 4-positions, or di-substituted by 4-iodo-3-nitro, 4-chloro-3-trifluoromethyl;
for group (c) the phenyl group is monosubstituted by NO₂, OH, I, F, Cl, OMe, N(R′)(R″) at any of the 2,3 or 4-positions, or di-substituted by 4-methyl-3-nitro, 4-fluoro-3-methyl, 3-iodo-4-methyl, 4-chloro-3-methyl, 4-iodo-3-nitro, 4-methly-3-nitro;
for group (d) the phenyl group is mono-substituted by chloro, bromo, iodo, fluoro, OH, nitro, CF₃ or OMe at any of the 2, 3 or 4 positions, or disubstituted by 4-hydroxy-3-nitro, 3-chloro-4-ethoxycarbonyl, 3,4-difluoro, 2,4-difluoro, 4-chloro-3-trifluoromethyl or 4-fluoro-3-nitro.

12. The method of claim 11, wherein for group (a) the phenyl group is monosubstituted by Br, I, NO₂, Cl, OMe, F, CN, OH or CF₃.

13. The method of claim 1, wherein said compound is of formula Ib, or a pharmaceutically acceptable salt thereof, 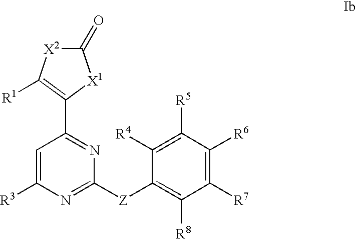
wherein one of X¹ and X² is S, and the other of X¹ and X² is NR⁹, and R¹⁻⁹ are as defined in claim 1.

14. The method of claim 13, wherein X¹ is S, X² is NR⁹, R⁹ is alkyl.

15. The method of claim 1, wherein said compound of formula I is selected from the the group consisting of:
1 (2-Chloro-phenyl)-[4-(2,4-dimethyl-thiazol-5-yl)-pyrimidin-2-yl]-amine
2 (4-Chloro-phenyl)-[4-(2,4-dimethyl-thiazol-5-yl)-pyrimidin-2-yl]-amine
3 (3-Chloro-phenyl)-[4-(2,4-dimethyl-thiazol-5-yl)-pyrimidin-2-yl]-amine
4 [4-(2,4-Dimethyl-thiazol-5-yl)-pyrimidin-2-yl]-(2-nitro-phenyl)-amine
5 [4-(2,4-Dimethyl-thiazol-5-yl)-pyrimidin-2-yl]-(3-nitro-phenyl)-amine
6 [4-(2,4-Dimethyl-thiazol-5-yl)-pyrimidin-2-yl]-(4-nitro-phenyl)-amine
7 [4-(2,4-Dimethyl-thiazol-5-yl)-pyrimidin-2-yl]-(2-fluoro-phenyl)-amine
8 [4-(2,4-Dimethyl-thiazol-5-yl)-pyrimidin-2-yl]-(4-fluoro-phenyl)-amine
9 (2,4-Difluoro-phenyl)-[4-(2,4-dimethyl-thiazol-5-yl)-pyrimidin-2-yl]-amine
10 (3,5-Difluoro-phenyl)-[4-(2,4-dimethyl-thiazol-5-yl)-pyrimidin-2-yl]-amine
11 (3,5-Dichloro-phenyl)-[4-(2,4-dimethyl-thiazol-5-yl)-pyrimidin-2-yl]-amine
12 (2,4-Dichloro-phenyl)-[4-(2,4-dimethyl-thiazol-5-yl)-pyrimidin-2-yl]-amine
13 [4-(2,4-Dimethyl-thiazol-5-yl)-pyrimidin-2-yl]-(3-trifluoromethyl-phenyl)-amine
14 [4-(2,4-Dimethyl-thiazol-5-yl)-pyrimidin-2-yl]-(2-trifluoromethyl-phenyl)-amine
15 [4-(2,4-Dimethyl-thiazol-5-yl)-pyrimidin-2-yl]-(4-trifluoromethyl-phenyl)-amine
16 (2-Bromo-phenyl)-[4-(2,4-dimethyl-thiazol-5-yl)-pyrimidin-2-yl]-amine
17 (3-Bromo-phenyl)-[4-(2,4-dimethyl-thiazol-5-yl)-pyrimidin-2-yl]-amine
18 (4-Bromo-phenyl)-[4-(2,4-dimethyl-thiazol-5-yl)-pyrimidin-2-yl]-amine
19 [4-(2,4-Dimethyl-thiazol-5-yl)-pyrimidin-2-yl]-(2-iodo-phenyl)-amine
20 [4-(2,4-Dimethyl-thiazol-5-yl)-pyrimidin-2-yl]-(3-iodo-phenyl)-amine
21 [4-(2,4-Dimethyl-thiazol-5-yl)-pyrimidin-2-yl]-(4-iodo-phenyl)-amine
22 [4-(2,4-Dimethyl-thiazol-5-yl)-pyrimidin-2-yl]-(3-fluoro-phenyl)-amine
23 (3,4-Difluoro-phenyl)-[4-(2,4-dimethyl-thiazol-5-yl)-pyrimidin-2-yl]-amine
24 [4-(2,4-Dimethyl-thiazol-5-yl)-pyrimidin-2-yl]-(2-methoxy-phenyl)-amine
25 [4-(2,4-Dimethyl-thiazol-5-yl)-pyrimidin-2-yl]-(3-methoxy-phenyl)-amine
26 [4-(2,4-Dimethyl-thiazol-5-yl)-pyrimidin-2-yl]-(4-methoxy-phenyl)-amine
27 3-[4-(2,4-Dimethyl-thiazol-5-yl)-pyrimidin-2-ylamino]-phenol
28 4-[4-(2,4-Dimethyl-thiazol-5-yl)-pyrimidin-2-ylamino]-phenol
29 N-[4-(2,4-Dimethyl-thiazol-5-yl)-pyrimidin-2-yl]-3-nitro-benzenesulfonamide
30 4-Chloro-N-[4-(2,4-dimethyl-thiazol-5-yl)-pyrimidin-2-yl]-benzenesulfonamide
31 N-[4-(2,4-Dimethyl-thiazol-5-yl)-pyrimidin-2-yl]-4-fluoro-benzenesulfonamide
32 4-[4-(2,4-Dimethyl-thiazol-5-yl)-pyrimidin-2-ylamino]-2-nitro-phenol
33 N-[4-(2,4-Dimethyl-thiazol-5-yl)-pyrimidin-2-yl]-4-nitro-benzenesulfonamide
34 N-[4-(2,4-Dimethyl-thiazol-5-yl)-pyrimidin-2-yl]-benzene-1,3-diamine
35 4-[4-(2,4-Dimethyl-thiazol-5-yl)-pyrimidin-2-ylamino]-benzonitrile
36 3-[4-(2,4-Dimethyl-thiazol-5-yl)-pyrimidin-2-ylamino]-benzonitrile
37 4-[4-(2,4-Dimethyl-thiazol-5-yl)-pyrimidin-2-ylamino]-benzoic acid methyl ester
38 (3-Chloro-4-methyl-phenyl)-[4-(2,4-dimethyl-thiazol-5-yl)-pyrimidin-2-yl]-amine
39 (3-Chloro-4-methoxy-phenyl)-[4-(2,4-dimethyl-thiazol-5-yl)-pyrimidin-2-yl]-amine
40 4-[4-(2,4-Dimethyl-thiazol-5-yl)-pyrimidin-2-ylamino]-benzoic acid
41 [4-Bromo-6-(2,4-dimethyl-thiazol-5-yl)-pyrimidin-2-yl]-(3-nitro-phenyl)-amine
42 [4-(2,4-Dimethyl-thiazol-5-yl)-6-phenyl-pyrimidin-2-yl]-(3-nitro-phenyl)-amine
43 4-[4-(2,4-Dimethyl-thiazol-5-yl)-6-phenyl-pyrimidin-2-ylamino]-phenol
44 (3,4-Difluoro-phenyl)-[4-(4-methyl-2-phenyl-thiazol-5-yl)-pyrimidin-2-yl]-amine
45 4-[4-(4-Methyl-2-phenyl-thiazol-5-yl)-pyrimidin-2-ylamino]-phenol
46 [4-(2,4-Dimethyl-thiazol-5-yl)-6-phenyl-pyrimidin-2-yl]-(4-fluoro-phenyl)-amine
47 (4-Fluoro-phenyl)-[4-(4-methyl-2-methylamino-thiazol-5-yl)-pyrimidin-2-yl]-amine
48 4-[4-(4-Methyl-2-methylamino-thiazol-5-yl)-pyrimidin-2-ylamino]-phenol
49 [4-(2,4-Dimethyl-thiazol-5-yl)-6-(4-trifluoromethyl-phenyl)-pyrimidin-2-yl]-(4-fluoro-phenyl)-amine
50 (4-Chloro-phenyl)-[4-(2,4-dimethyl-thiazol-5-yl)-6-(4-trifluoromethyl-phenyl)-pyrimidin-2-yl]-amine
51 4-[4-(2,4-Dimethyl-thiazol-5-yl)-6-(4-trifluoromethyl-phenyl)-pyrimidin-2-ylamino]-2-nitro-phenol
52 (4-Fluoro-phenyl)-[4-(4-methyl-2-pyridin-3-yl-thiazol-5-yl)-pyrimidin-2-yl]-amine
53 [4-(2,4-Dimethyl-thiazol-5-yl)-6-(3-trifluoromethyl-phenyl)-pyrimidin-2-yl]-(4-fluoro-phenyl)-amine
54 4-[6-(2,4-Dimethyl-thiazol-5-yl)-2-(4-fluoro-phenylamino)-pyrimidin-4-yl]-2,6-dimethoxy-phenol
55 4-[6-(2,4-Dimethyl-thiazol-5-yl)-2-(4-fluoro-phenylamino)-pyrimidin-4-yl]-phenol
56 [4-(4-Methyl-2-pyridin-3-yl-thiazol-5-yl)-pyrimidin-2-yl]-(3-nitro-phenyl)-amine
57 (4-Iodo-phenyl)-[4-(4-methyl-2-methylamino-thiazol-5-yl)-pyrimidin-2-yl]-amine
58 4-[4-(4-Methyl-2-methylamino-thiazol-5-yl)-pyrimidin-2-ylamino]-2-nitro-phenol
59 2-Chloro-4-[4-(4-methyl-2-methylamino-thiazol-5-yl)-pyrimidin-2-ylamino]-benzoic acid ethyl ester
60 [4-(2-Ethylamino-4-methyl-thiazol-5-yl)-pyrimidin-2-yl]-(4-fluoro-phenyl)-amine
61 [4-(4-Methyl-2-methylamino-thiazol-5-yl)-pyrimidin-2-yl]-(3-nitro-phenyl)-amine
62 3-[4-(2-Ethylamino-4-methyl-thiazol-5-yl)-pyrimidin-2-ylamino]-phenol
63 2-Chloro-4-[4-(2-ethylamino-4-methyl-thiazol-5-yl)-pyrimidin-2-ylamino]-benzoic acid ethyl ester
64 4-Chloro-3-[4-(2,4-dimethyl-thiazol-5-yl)-pyrimidin-2-ylamino]-benzoic acid 2-methoxy-ethyl ester
65 2-Chloro-4-[4-(2,4-dimethyl-thiazol-5-yl)-pyrimidin-2-ylamino]-benzoic acid 2-methoxy-ethyl ester
66 4-Chloro-3-[4-(2,4-dimethyl-thiazol-5-yl)-pyrimidin-2-ylamino]-benzoic acid
67 [4-(2-Allylamino-4-methyl-thiazol-5-yl)-pyrimidin-2-yl]-(4-fluoro-phenyl)-amine
68 (3-Bromo-phenyl)-[4-(4-methyl-2-methylamino-thiazol-5-yl)-pyrimidin-2-yl]-amine
69 [4-(2-Allylamino-4-methyl-thiazol-5-yl)-pyrimidin-2-yl]-(3-nitro-phenyl)-amine
70 3-[4-(4-Methyl-2-methylamino-thiazol-5-yl)-pyrimidin-2-ylamino]-phenol
71 (4-Bromo-phenyl)-[4-(4-methyl-2-methylamino-thiazol-5-yl)-pyrimidin-2-yl]-amine
72 (4-Chloro-phenyl)-[4-(4-methyl-2-methylamino-thiazol-5-yl)-pyrimidin-2-yl]-amine
73 (3-Methoxy-phenyl)-[4-(4-methyl-2-methylamino-thiazol-5-yl)-pyrimidin-2-yl]-amine
74 [4-(4-Methyl-2-methylamino-thiazol-5-yl)-pyrimidin-2-yl]-(4-trifluoromethyl-phenyl)-amine
75 [4-(4-Methyl-2-methylamino-thiazol-5-yl)-pyrimidin-2-yl]-(3-trifluoromethyl-phenyl)-amine
76 2-Chloro-5-[4-(2,4-dimethyl-thiazol-5-yl)-pyrimidin-2-ylamino]-benzoic acid ethyl ester
77 3-Chloro-2-[4-(2,4-dimethyl-thiazol-5-yl)-pyrimidin-2-ylamino]-benzoic acid ethyl ester
78 [4-(2,4-Dimethyl-thiazol-5-yl)-pyrimidin-2-yl]-(2-fluoro-4-iodo-phenyl)-amine
79 2-[4-(2,4-Dimethyl-thiazol-5-yl)-pyrimidin-2-ylamino]-5-methoxy-phenol
80 (3-Chloro-4-iodo-phenyl)-[4-(2,4-dimethyl-thiazol-5-yl)-pyrimidin-2-yl]-amine
81 2-Chloro-4-[4-(2,4-dimethyl-thiazol-5-yl)-pyrimidin-2-ylamino]-phenol
82 5-[4-(2,4-Dimethyl-thiazol-5-yl)-pyrimidin-2-ylamino]-2-fluoro-benzoic acid 2-methoxy-ethyl ester
83 2-Chloro-5-[4-(2,4-dimethyl-thiazol-5-yl)-pyrimidin-2-ylamino]-benzoic acid methyl ester
84 4-Chloro-3-[4-(2,4-dimethyl-thiazol-5-yl)-pyrimidin-2-ylamino]-benzoic acid methyl ester
85 2-Chloro-4-[4-(2,4-dimethyl-thiazol-5-yl)-pyrimidin-2-ylamino]-benzoic acid methyl ester
86 (3-Iodo-phenyl)-[4-(4-methyl-2-methylamino-thiazol-5-yl)-pyrimidin-2-yl]-amine
87 (3-Fluoro-phenyl)-[4-(4-methyl-2-methylamino-thiazol-5-yl)-pyrimidin-2-yl]-amine
88 (3,4-Difluoro-phenyl)-[4-(4-methyl-2-methylamino-thiazol-5-yl)-pyrimidin-2-yl]-amine
89 (2,4-Difluoro-phenyl)-[4-(4-methyl-2-methylamino-thiazol-5-yl)-pyrimidin-2-yl]-amine
90 (3,5-Difluoro-phenyl)-[4-(4-methyl-2-methylamino-thiazol-5-yl)-pyrimidin-2-yl]-amine
91 (4-Chloro-3-trifluoromethyl-phenyl)-[4-(4-methyl-2-methylamino-thiazol-5-yl)-pyrimidin-2-yl]-amine
92 (3-Chloro-phenyl)-[4-(4-methyl-2-methylamino-thiazol-5-yl)-pyrimidin-2-yl]-amine
93 (4-Methoxy-phenyl)-[4-(4-methyl-2-methylamino-thiazol-5-yl)-pyrimidin-2-yl]-amine
94(4-Fluoro-3-nitro-phenyl)-[4-(4-methyl-2-methylamino-thiazol-5-yl)-pyrimidin-2-yl]-amine
95 4-{4-[2-(4-Nitro-phenylamino)-thiazol-5-yl]-pyrimidin-2-ylamino}-phenol
96 N-{5-[2-(4-Hydroxy-phenylamino)-pyrimidin-4-yl]-4-methyl-thiazol-2-yl}-acetamide
97 (4-Fluoro-phenyl)-{4-[2-(4-nitro-phenylamino)-thiazol-5-yl]-pyrimidin-2-yl}-amine
98 4-[4-(2-Amino-4-methyl-thiazol-5-yl)-pyrimidin-2-ylamino]-phenol
99 N-{3-[4-(2,4-Dimethyl-thiazol-5-yl)-pyrimidin-2-ylamino]-phenyl}-guanidine
100 {3-[4-(2,4-Dimethyl-thiazol-5-yl)-pyrimidin-2-ylamino]-phenyl}-methanol
101 [4-(2,4-Dimethyl-thiazol-5-yl)-pyrimidin-2-yl]-(4-pyridin-4-ylmethyl-phenyl)-amine
102 [3-(2-Diethylamino-ethoxymethyl)-phenyl]-[4-(2,4-dimethyl-thiazol-5-yl)-pyrimidin-2-yl]-amine
103 N,N-Dimethyl-N′-[4-(2,4-dimethyl-thiazol-5-yl)-pyrimidin-2-yl]-benzene-1,4-diamine
104 {4-[4-(2,4-Dimethyl-thiazol-5-yl)-pyrimidin-2-ylamino]-phenyl}-trimethyl-ammonium
105 [4-(2-Amino-4-methyl-thiazol-5-yl)-pyrimidin-2-yl]-(3-nitro-phenyl)-amine
106 N-[4-(2-Amino-4-methyl-thiazol-5-yl)-pyrimidin-2-yl]-N′,N′-dimethyl-benzene-1,4-diamine
107 [4-(2-Amino-4-methyl-thiazol-5-yl)-pyrimidin-2-yl]-(4-chloro-phenyl)-amine
108 [4-(2-Amino-4-methyl-thiazol-5-yl)-pyrimidin-2-yl]-(3-methoxy-phenyl)-amine
109 [4-(2-Amino-4-methyl-thiazol-5-yl)-pyrimidin-2-yl]-(3-fluoro-phenyl)-amine
110 [4-(2-Amino-4-methyl-thiazol-5-yl)-pyrimidin-2-yl]-(4-trifluoromethyl-phenyl)-amine
111 [4-(2-Amino-4-methyl-thiazol-5-yl)-pyrimidin-2-yl]-(4-methoxy-phenyl)-amine
112 [4-(2-Amino-4-methyl-thiazol-5-yl)-pyrimidin-2-yl]-(3-chloro-phenyl)-amine
113 [4-(2-Amino-4-methyl-thiazol-5-yl)-pyrimidin-2-yl]-(3-iodo-phenyl)-amine
114 [4-(2-Amino-4-methyl-thiazol-5-yl)-pyrimidin-2-yl]-(4-iodo-phenyl)-amine
115 [4-(2-Amino-4-methyl-thiazol-5-yl)-pyrimidin-2-yl]-(4-fluoro-phenyl)-amine
116 3-[4-(2-Amino-4-methyl-thiazol-5-yl)-pyrimidin-2-ylamino]-phenol
117 [4-(2-Amino-4-methyl-thiazol-5-yl)-pyrimidin-2-yl]-(4-iodo-3-nitro-phenyl)-amine
118 2-{4-[4-(2-Amino-4-methyl-thiazol-5-yl)-pyrimidin-2-ylamino]-phenyl}-ethanol
119 [4-(2-Amino-4-methyl-thiazol-5-yl)-pyrimidin-2-yl]-(3-bromo-phenyl)-amine
120 [4-(2-Amino-4-methyl-thiazol-5-yl)-pyrimidin-2-yl]-(4-bromo-phenyl)-amine
121 [4-(2-Amino-4-methyl-thiazol-5-yl)-pyrimidin-2-yl]-(4-chloro-3-trifluoromethyl-phenyl)-amine
122 [4-(2-Diethylamino-ethoxy)-phenyl]-[4-(2,4-dimethyl-thiazol-5-yl)-pyrimidin-2-yl]-amine
123 2-{4-[4-(2,4-Dimethyl-thiazol-5-yl)-pyrimidin-2-ylamino]-phenyl}-ethanol
124 2-({4-[4-(2,4-Dimethyl-thiazol-5-yl)-pyrimidin-2-ylamino]-phenyl}-ethyl-amino)-ethanol
125 (3,4-Dimethoxy-phenyl)-[4-(2,4-dimethyl-thiazol-5-yl)-pyrimidin-2-yl]-amine
126 5-[4-(2,4-Dimethyl-thiazol-5-yl)-pyrimidin-2-ylamino]-2-methoxy-phenol
127 N⁴-[4-(2,4-Dimethyl-thiazol-5-yl)-pyrimidin-2-yl]-N¹,N¹-dimethyl-2-nitro-benzene-1,4-diamine
128 2-Chloro-N⁴-[4-(2,4-dimethyl-thiazol-5-yl)-pyrimidin-2-yl]-N¹,N¹-dimethyl-benzene-1,4-diamine
129 N⁴-[4-(2,4-Dimethyl-thiazol-5-yl)-pyrimidin-2-yl]-N¹,N¹-dimethyl-2-trifluoromethyl-benzene-1,4-diamine
130 N¹-[4-(2,4-Dimethyl-thiazol-5-yl)-pyrimidin-2-yl]-4-methoxy-N³,N³-dimethyl-benzene-1,3-diamine
131 N,N-Dimethyl-N′-[4-(4-methyl-2-methylamino-thiazol-5-yl)-pyrimidin-2-yl]-benzene- 1,4-diamine
132 (4-Iodo-3-nitro-phenyl)-[4-(4-methyl-2-methylamino-thiazol-5-yl)-pyrimidin-2-yl]-amine
133 [4-(2-Ethylamino-4-methyl-thiazol-5-yl)-pyrimidin-2-yl]-(3-nitro-phenyl)-amine;
134 (4-Chloro-phenyl)-[4-(2-ethylamino-4-methyl-thiazol-5-yl)-pyrimidin-2-yl]-amine;
135 [4-(2-Ethylamino-4-methyl-thiazol-5-yl)-pyrimidin-2-yl]-(4-trifluoromethyl-phenyl)-amine
136 [4-(2-Ethylamino-4-methyl-thiazol-5-yl)-pyrimidin-2-yl]-(3-methoxy-phenyl)-amine
137 (3-Chloro-phenyl)-[4-(2-ethylamino-4-methyl-thiazol-5-yl)-pyrimidin-2-yl]-amine
138 [4-(2-Ethylamino-4-methyl-thiazol-5-yl)-pyrimidin-2-yl]-(4-methyl-3-nitro-phenyl)-amine
139 [4-(2-Butylamino-4-methyl-thiazol-5-yl)-pyrimidin-2-yl]-(4-fluoro-phenyl)-amine
140 [4-(2-Dimethylamino-4-methyl-thiazol-5-yl)-pyrimidin-2-yl]-(3-nitro-phenyl)-amine
141 (4-Chloro-phenyl)-[4-(2-dimethylamino-4-methyl-thiazol-5-yl)-pyrimidin-2-yl]-amine
142 [4-(2-Dimethylamino-4-methyl-thiazol-5-yl)-pyrimidin-2-yl]-(4-fluoro-phenyl)-amine
143 (3-Chloro-phenyl)-[4-(2-dimethylamino-4-methyl-thiazol-5-yl)-pyrimidin-2-yl]-amine
144 2-{4-Methyl-5-[2-(3-nitro-phenylamino)-pyrimidin-4-yl]-thiazol-2-ylamino}-ethanol
145 2-{5-[2-(4-Fluoro-phenylamino)-pyrimidin-4-yl]-4-methyl-thiazol-2-ylamino}-ethanol
146 [4-(2-Amino-4-methyl-thiazol-5-yl)-pyrimidin-2-yl]-phenyl-amine
147 4-[4-(2-Amino-4-methyl-thiazol-5-yl)-pyrimidin-2-ylamino]-benzenesulfonic acid
148 4-[4-(2,4-Dimethyl-thiazol-5-yl)-pyrimidin-2-ylamino]-benzenesulfonic acid
149 N-[4-(2-Amino-4-methyl-thiazol-5-yl)-pyrimidin-2-yl]-benzene-1,3-diamine
150 [4-(2,4-Dimethyl-thiazol-5-yl)-pyrimidin-2-yl]-phenyl-amine
151 [4-(2-Amino-4-methyl-thiazol-5-yl)-pyrimidin-2-yl]-(4-nitro-phenyl)-amine
152 5-[2-(4-Hydroxy-phenylamino)-pyrimidin-4-yl]-3,4-dimethyl-3H-thiazol-2-one
153 3,4-Dimethyl-5-[2-(3-nitro-phenylamino)-pyrimidin-4-yl]-3H-thiazol-2-one
154 5-[2-(4-Iodo-phenylamino)-pyrimidin-4-yl]-3,4-dimethyl-3H-thiazol-2-one
155 5-[2-(4-Fluoro-phenylamino)-pyrimidin-4-yl]-3,4-dimethyl-3H-thiazol-2-one
156 5-[2-(4-Chloro-phenylamino)-pyrimidin-4-yl]-3,4-dimethyl-3H-thiazol-2-one
157 5-[2-(4-Methoxy-phenylamino)-pyrimidin-4-yl]-3,4-dimethyl-3H-thiazol-2-one
158 5-[2-(3-Hydroxy-phenylamino)-pyrimidin-4-yl]-3,4-dimethyl-3H-thiazol-2-one
159 5-[2-(4-Fluoro-3-nitro-phenylamino)-pyrimidin-4-yl]-3,4-dimethyl-3H-thiazol-2-one
160 5-[2-(4-Chloro-3-methyl-phenylamino)-pyrimidin-4-yl]-3,4-dimethyl-3H-thiazol-2-one
161 5-[2-(3-Iodo-4-methyl-phenylamino)-pyrimidin-4-yl]-3,4-dimethyl-3H-thiazol-2-one
162 5-[2-(4-Fluoro-3-methyl-phenylamino)-pyrimidin-4-yl]-3,4-dimethyl-3H-thiazol-2-one
163 3,4-Dimethyl-5-[2-(4-methyl-3-nitro-phenylamino)-pyrimidin-4-yl]-3H-thiazol-2-one
164 5-[2-(4-Dimethylamino-phenylamino)-pyrimidin-4-yl]-3,4-dimethyl-3H-thiazol-2-one

16. The method of claim 15, wherein said compound of formula I is selected from the group consisting of:
N-[4-(2,4-Dimethyl-thiazol-5-yl)-pyrimidin-2-yl]-N′,N′-dimethyl-benzene-1,4-diamine [103];
N⁴-[4-(2,4-Dimethyl-thiazol-5-yl)-pyrimidin-2-yl]-N¹,N¹-dimethyl-2-nitro-benzene-1,4-diamine [127];
[4-(4-Methyl-2-methylamino-thiazol-5-yl)-pyrimidin-2-yl]-(3-nitro-phenyl)-amine [61];
3-[4-(4-Methyl-2-methylamino-thiazol-5-yl)-pyrimidin-2-ylamino]-phenol [62];
[4-(2,4-Dimethyl-thiazol-5-yl)-pyrimidin-2-yl]-(3-nitro-phenyl)-amine [5];
4-[4-(2,4-Dimethyl-thiazol-5-yl)-pyrimidin-2-ylamino]-2-nitro-phenol [32];
(4-Fluoro-phenyl)-[4-(4-methyl-2-methylamino-thiazol-5-yl)-pyrimidin-2-yl]-amine [47];
(3-Methoxy-phenyl)-[4-(4-methyl-2-methylamino-thiazol-5-yl)-pyrimidin-2-yl]-amine [73];
[4-(2-Amino-4-methyl-thiazol-5-yl)-pyrimidin-2-yl]-(3-nitro-phenyl)-amine [105];
3-[4-(2-Amino-4-methyl-thiazol-5-yl)-pyrimidin-2-ylamino]-phenol [116];
[4-(2-Methoxy-4-methyl-thiazol-5-yl)-pyrimidin-2-yl]-(4-methyl-3-nitro-phenyl)-amine [144];
(4-Fluoro-3-methyl-phenyl)-[4-(2-methoxy-4-methyl-thiazol-5-yl)-pyrimidin-2-yl]-amine [143];
[4-(2-Ethylamino-4-methyl-thiazol-5-yl)-pyrimidin-2-yl]-(3-nitro-phenyl)-amine [133]
[4-(2-Ethylamino-4-methyl-thiazol-5-yl)-pyrimidin-2-yl]-(4-fluoro-phenyl)-amine [60];
(3-Iodo-4-methyl-phenyl)-[4-(2-methoxy-4-methyl-thiazol-5-yl)-pyrimidin-2-yl]-amine [142];
(4-Chloro-3-methyl-phenyl)-[4-(2-methoxy-4-methyl-thiazol-5-yl)-pyrimidin-2-yl]-amine [141];
(3,4-Dimethoxy-phenyl)-[4-(2,4-dimethyl-thiazol-5-yl)-pyrimidin-2-yl]-amine [125];
5-[4-(2,4-Dimethyl-thiazol-5-yl)-pyrimidin-2-ylamino]-2-methoxy-phenol [126];
N-[4-(2,4-Dimethyl-thiazol-5-yl)-pyrimidin-2-yl]-benzene-1,3-diamine [34];
[4-(2,4-Dimethyl-thiazol-5-yl)-pyrimidin-2-yl]-phenyl-amine [150];
N-[4-(2-Amino-4-methyl-thiazol-5-yl)-pyrimidin-2-yl]-benzene-1,3-diamine [149];
3-[4-(2,4-Dimethyl-thiazol-5-yl)-pyrimidin-2-ylamino]-phenol [27];
4-[4-(2,4-Dimethyl-thiazol-5-yl)-pyrimidin-2-ylamino]-phenol [28]; and
4-[4-(4-Methyl-2-methylamino-thiazol-5-yl)-pyrimidin-2-ylamino]-phenol [48].

17. Use according to claim 15 or claim 16 wherein said compound of formula I selected from the following:
(4-Chloro-phenyl)-[4-(2,4-dimethyl-thiazol-5-yl)-pyrimidin-2-yl]-amine [2];
(3-Chloro-phenyl)-[4-(2,4-dimethyl-thiazol-5-yl)-pyrimidin-2-yl]-amine [3];
[4-(2,4-Dimethyl-thiazol-5-yl)-pyrimidin-2-yl]-(3-nitro-phenyl)-amine [5];
[4-(2,4-Dimethyl-thiazol-5-yl)-pyrimidin-2-yl]-(4-nitro-phenyl)-amine [6];
[4-(2,4-Dimethyl-thiazol-5-yl)-pyrimidin-2-yl]-(2-fluoro-phenyl)-amine [7];
[4-(2,4-Dimethyl-thiazol-5-yl)-pyrimidin-2-yl]-(4-fluoro-phenyl)-amine [8];
(2,4-Difluoro-phenyl)-[4-(2,4-dimethyl-thiazol-5-yl)-pyrimidin-2-yl]-amine [9];
(3,5-Difluoro-phenyl)-[4-(2,4-dimethyl-thiazol-5-yl)-pyrimidin-2-yl]-amine [10];
(3,5-Dichloro-phenyl)-[4-(2,4-dimethyl-thiazol-5-yl)-pyrimidin-2-yl]-amine [11];
(2,4-Dichloro-phenyl)-[4-(2,4-dimethyl-thiazol-5-yl)-pyrimidin-2-yl]-amine [12];
[4-(2,4-Dimethyl-thiazol-5-yl)-pyrimidin-2-yl]-(4-trifluoromethyl-phenyl)-amine [15];
(3-Bromo-phenyl)-[4-(2,4-dimethyl-thiazol-5-yl)-pyrimidin-2-yl]-amine [17];
[4-(2,4-Dimethyl-thiazol-5-yl)-pyrimidin-2-yl]-(3-iodo-phenyl)-amine [20];
[4-(2,4-Dimethyl-thiazol-5-yl)-pyrimidin-2-yl]-(3-fluoro-phenyl)-amine [22];
(3,4-Difluoro-phenyl)-[4-(2,4-dimethyl-thiazol-5-yl)-pyrimidin-2-yl]-amine [23];
[4-(2,4-Dimethyl-thiazol-5-yl)-pyrimidin-2-yl]-(2-methoxy-phenyl)-amine [24];
[4-(2,4-Dimethyl-thiazol-5-yl)-pyrimidin-2-yl]-(3-methoxy-phenyl)-amine [25];
[4-(2,4-Dimethyl-thiazol-5-yl)-pyrimidin-2-yl]-(4-methoxy-phenyl)-amine [26];
3-[4-(2,4-Dimethyl-thiazol-5-yl)-pyrimidin-2-ylamino]-phenol [27];
4-[4-(2,4-Dimethyl-thiazol-5-yl)-pyrimidin-2-ylamino]-phenol [28];
4-[4-(2,4-Dimethyl-thiazol-5-yl)-pyrimidin-2-ylamino]-2-nitro-phenol [32];
N-[4-(2,4-Dimethyl-thiazol-5-yl)-pyrimidin-2-yl]-benzene-1,3-diamine [34];
4-[4-(2,4-Dimethyl-thiazol-5-yl)-pyrimidin-2-ylamino]-benzonitrile [35];
3-[4-(2,4-Dimethyl-thiazol-5-yl)-pyrimidin-2-ylamino]-benzonitrile [36];
4-[4-(2,4-Dimethyl-thiazol-5-yl)-pyrimidin-2-ylamino]-benzoic acid methyl ester [37];
(3-Chloro-4-methoxy-phenyl)-[4-(2,4-dimethyl-thiazol-5-yl)-pyrimidin-2-yl]-amine [39];
4-[4-(2,4-Dimethyl-thiazol-5-yl)-pyrimidin-2-ylamino]-benzoic acid [40];
[4-Bromo-6-(2,4-dimethyl-thiazol-5-yl)-pyrimidin-2-yl]-(3-nitro-phenyl)-amine [41];
(4-Fluoro-phenyl)-[4-(4-methyl-2-methylamino-thiazol-5-yl)-pyrimidin-2-yl]-amine [47];
4-[4-(4-Methyl-2-methylamino-thiazol-5-yl)-pyrimidin-2-ylamino]-phenol [48];
4-[4-(4-Methyl-2-methylamino-thiazol-5-yl)-pyrimidin-2-ylamino]-2-nitro-phenol [58];
[4-(2-Ethylamino-4-methyl-thiazol-5-yl)-pyrimidin-2-yl]-(4-fluoro-phenyl)-amine [60];
[4-(4-Methyl-2-methylamino-thiazol-5-yl)-pyrimidin-2-yl]-(3-nitro-phenyl)-amine [61];
[4-(2-Allylamino-4-methyl-thiazol-5-yl)-pyrimidin-2-yl]-(4-fluoro-phenyl)-amine [67];
(3-Bromo-phenyl)-[4-(4-methyl-2-methylamino-thiazol-5-yl)-pyrimidin-2-yl]-amine [68];
[4-(2-Allylamino-4-methyl-thiazol-5-yl)-pyrimidin-2-yl]-(3-nitro-phenyl)-amine [69];
3-[4-(4-Methyl-2-methylamino-thiazol-5-yl)-pyrimidin-2-ylamino]-phenol [70];
(4-Chloro-phenyl)-[4-(4-methyl-2-methylamino-thiazol-5-yl)-pyrimidin-2-yl]-amine [72];
(3-Methoxy-phenyl)-[4-(4-methyl-2-methylamino-thiazol-5-yl)-pyrimidin-2-yl]-amine [73];
[4-(4-Methyl-2-methylamino-thiazol-5-yl)-pyrimidin-2-yl]-(4-trifluoromethyl-phenyl)-amine [74];
[4-(4-Methyl-2-methylamino-thiazol-5-yl)-pyrimidin-2-yl]-(3-trifluoromethyl-phenyl)-amine [75];
2-[4-(2,4-Dimethyl-thiazol-5-yl)-pyrimidin-2-ylamino]-5-methoxy-phenol [79];
2-Chloro-5-[4-(2,4-dimethyl-thiazol-5-yl)-pyrimidin-2-ylamino]-benzoic acid methyl ester; [83];
(3-Fluoro-phenyl)-[4-(4-methyl-2-methylamino-thiazol-5-yl)-pyrimidin-2-yl]-amine [87];
(4-Methoxy-phenyl)-[4-(4-methyl-2-methylamino-thiazol-5-yl)-pyrimidin-2-yl]-amine [93];
4-{4-[2-(4-Nitro-phenylamino)-thiazol-5-yl]-pyrimidin-2-ylamino}-phenol [95];
4-[4-(2-Amino-4-methyl-thiazol-5-yl)-pyrimidin-2-ylamino]-phenol [98];
N-{3-[4-(2,4-Dimethyl-thiazol-5-yl)-pyrimidin-2-ylamino]-phenyl}-guanidine [99];
{3-[4-(2,4-Dimethyl-thiazol-5-yl)-pyrimidin-2-ylamino]-phenyl}-methanol [100];
[4-(2,4-Dimethyl-thiazol-5-yl)-pyrimidin-2-yl]-(4-pyridin-4-ylmethyl-phenyl)-amine [101];
N,N-Dimethyl-N′-[4-(2,4-dimethyl-thiazol-5-yl)-pyrimidin-2-yl]-benzene-1,4-diamine [103];
{4-[4-(2,4-Dimethyl-thiazol-5-yl)-pyrimidin-2-ylamino]-phenyl}-trimethyl-ammonium [104];
[4-(2-Amino-4-methyl-thiazol-5-yl)-pyrimidin-2-yl]-(3-nitro-phenyl)-amine [105];
N-[4-(2-Amino-4-methyl-thiazol-5-yl)-pyrimidin-2-yl]-N′,N′-dimethyl-benzene-1,4-diamine [106];
[4-(2-Amino-4-methyl-thiazol-5-yl)-pyrimidin-2-yl]-(3-methoxy-phenyl)-amine [108];
[4-(2-Amino-4-methyl-thiazol-5-yl)-pyrimidin-2-yl]-(3-fluoro-phenyl)-amine [109];
[4-(2-Amino-4-methyl-thiazol-5-yl)-pyrimidin-2-yl]-(4-trifluoromethyl-phenyl)-amine [110];
[4-(2-Amino-4-methyl-thiazol-5-yl)-pyrimidin-2-yl]-(4-methoxy-phenyl)-amine [111];
[4-(2-Amino-4-methyl-thiazol-5-yl)-pyrimidin-2-yl]-(3-chloro-phenyl)-amine [112];
[4-(2-Amino-4-methyl-thiazol-5-yl)-pyrimidin-2-yl]-(3-iodo-phenyl)-amine [113];
3-[4-(2-Amino-4-methyl-thiazol-5-yl)-pyrimidin-2-ylamino]-phenol [116];
[4-(2-Amino-4-methyl-thiazol-5-yl)-pyrimidin-2-yl]-(4-iodo-3-nitro-phenyl)-amine [117];
2-{4-[4-(2-Amino-4-methyl-thiazol-5-yl)-pyrimidin-2-ylamino]-phenyl}-ethanol [118];
[4-(2-Amino-4-methyl-thiazol-5-yl)-pyrimidin-2-yl]-(3-bromo-phenyl)-amine [119];
[4-(2-Amino-4-methyl-thiazol-5-yl)-pyrimidin-2-yl]-(4-bromo-phenyl)-amine [120];
N¹-[4-(2,4-Dimethyl-thiazol-5-yl)-pyrimidin-2-yl]-4-[P-(phenoxy)-triethylamine]-amine [122];
2-{4-[4-(2,4-Dimethyl-thiazol-5-yl)-pyrimidin-2-ylamino]-phenyl}-ethanol [123];
2-({4-[4-(2,4-Dimethyl-thiazol-5-yl)-pyrimidin-2-ylamino]-phenyl}-ethyl-amino)-ethanol [124];
(3,4-Dimethoxy-phenyl)-[4-(2,4-dimethyl-thiazol-5-yl)-pyrimidin-2-yl]-amine [125];
5-[4-(2,4-Dimethyl-thiazol-5-yl)-pyrimidin-2-ylamino]-2-methoxy-phenol [126];
N⁴-[4-(2,4-Dimethyl-thiazol-5-yl)-pyrimidin-2-yl]-N¹,N¹-dimethyl-2-nitro-benzene-1,4-diamine [127];
2-[N-(4-N,N-Dimethylamino-3-chlorophenyl)]-4-(2,4-dimethylthiazol-5-yl)-pyrimidineamine [128];
N¹-[4-(2,4-Dimethyl-thiazol-5-yl)-pyrimidin-2-yl]-4-methoxy-N³,N³-dimethyl-benzene-1,3-diamine [130];
N,N-Dimethyl-N′-[4-(4-methyl-2-methylamino-thiazol-5-yl)-pyrimidin-2-yl]-benzene-1,4-diamine [131];
(4-Iodo-3-nitro-phenyl)-[4-(4-methyl-2-methylamino-thiazol-5-yl)-pyrimidin-2-yl]-amine [132];
[4-(2-Ethylamino-4-methyl-thiazol-5-yl)-pyrimidin-2-yl]-(3-nitro-phenyl)-amine [133]
(4-Chloro-phenyl)-[4-(2-ethylamino-4-methyl-thiazol-5-yl)-pyrimidin-2-yl]-amine [134];
[4-(2-Ethylamino-4-methyl-thiazol-5-yl)-pyrimidin-2-yl]-(3-methoxy-phenyl)-amine [136];
[4-(2-Ethylamino-4-methyl-thiazol-5-yl)-pyrimidin-2-yl]-(4-methyl-3-nitro-phenyl)-amine [138];
[4-(2-Butylamino-4-methyl-thiazol-5-yl)-pyrimidin-2-yl]-(4-fluoro-phenyl)-amine [139];
[4-(2-Dimethylamino-4-methyl-thiazol-5-yl)-pyrimidin-2-yl]-(3-nitro-phenyl)-amine [140];
(4-Chloro-phenyl)-[4-(2-dimethylamino-4-methyl-thiazol-5-yl)-pyrimidin-2-yl]-amine [141];
[4-(2-Dimethylamino-4-methyl-thiazol-5-yl)-pyrimidin-2-yl]-(4-fluoro-phenyl)-amine [142];
(3-Chloro-phenyl)-[4-(2-dimethylamino-4-methyl-thiazol-5-yl)-pyrimidin-2-yl]-amine [143];
2-{4-Methyl-5-[2-(3-nitro-phenylamino)-pyrimidin-4-yl]-thiazol-2-ylamino}-ethanol [144];
2-{5-[2-(4-Fluoro-phenylamino)-pyrimidin-4-yl]-4-methyl-thiazol-2-ylamino}-ethanol [145];
4-[4-(2,4-Dimethyl-thiazol-5-yl)-pyrimidin-2-ylamino]-benzenesulfonic acid [148];
N-[4-(2-Amino-4-methyl-thiazol-5-yl)-pyrimidin-2-yl]-benzene-1,3-diamine [149].
[4-(2,4-Dimethyl-thiazol-5-yl)-pyrimidin-2-yl]-phenyl-amine [150]; and
[4-(2-Amino-4-methyl-thiazol-5-yl)-pyrimidin-2-yl]-(4-nitro-phenyl)-amine [151];

18. The method of claim 17, wherein the compound of formula I selected from the group consisting of:
[4-(2,4-Dimethyl-thiazol-5-yl)-pyrimidin-2-yl]-(3-nitro-phenyl)-amine [5];
[4-(2,4-Dimethyl-thiazol-5-yl)-pyrimidin-2-yl]-(4-fluoro-phenyl)-amine [8];
(2,4-Difluoro-phenyl)-[4-(2,4-dimethyl-thiazol-5-yl)-pyrimidin-2-yl]-amine [9];
[4-(2,4-Dimethyl-thiazol-5-yl)-pyrimidin-2-yl]-(4-trifluoromethyl-phenyl)-amine [15];
(3-Bromo-phenyl)-[4-(2,4-dimethyl-thiazol-5-yl)-pyrimidin-2-yl]-amine [17];
[4-(2,4-Dimethyl-thiazol-5-yl)-pyrimidin-2-yl]-(3-fluoro-phenyl)-amine [22];
(3,4-Difluoro-phenyl)-[4-(2,4-dimethyl-thiazol-5-yl)-pyrimidin-2-yl]-amine [23];
[4-(2,4-Dimethyl-thiazol-5-yl)-pyrimidin-2-yl]-(3-methoxy-phenyl)-amine [25];
[4-(2,4-Dimethyl-thiazol-5-yl)-pyrimidin-2-yl]-(4-methoxy-phenyl)-amine [26];
3-[4-(2,4-Dimethyl-thiazol-5-yl)-pyrimidin-2-ylamino]-phenol [27];
4-[4-(2,4-Dimethyl-thiazol-5-yl)-pyrimidin-2-ylamino]-phenol [28];
4-[4-(2,4-Dimethyl-thiazol-5-yl)-pyrimidin-2-ylamino]-2-nitro-phenol [32];
N-[4-(2,4-Dimethyl-thiazol-5-yl)-pyrimidin-2-yl]-benzene-1,3-diamine [34];
3-[4-(2,4-Dimethyl-thiazol-5-yl)-pyrimidin-2-ylamino]-benzonitrile [36];
4-[4-(2,4-Dimethyl-thiazol-5-yl)-pyrimidin-2-ylamino]-benzoic acid [40];
[4-Bromo-6-(2,4-dimethyl-thiazol-5-yl)-pyrimidin-2-yl]-(3-nitro-phenyl)-amine [41];
(4-Fluoro-phenyl)-[4-(4-methyl-2-methylamino-thiazol-5-yl)-pyrimidin-2-yl]-amine [47];
4-[4-(4-Methyl-2-methylamino-thiazol-5-yl)-pyrimidin-2-ylamino]-phenol [48];
4-[4-(4-Methyl-2-methylamino-thiazol-5-yl)-pyrimidin-2-ylamino]-2-nitro-phenol [58];
[4-(2-Ethylamino-4-methyl-thiazol-5-yl)-pyrimidin-2-yl]-(4-fluoro-phenyl)-amine [60];
[4-(4-Methyl-2-methylamino-thiazol-5-yl)-pyrimidin-2-yl]-(3-nitro-phenyl)-amine [61];
(3-Bromo-phenyl)-[4-(4-methyl-2-methylamino-thiazol-5-yl)-pyrimidin-2-yl]-amine [68];
[4-(2-Allylamino-4-methyl-thiazol-5-yl)-pyrimidin-2-yl]-(3-nitro-phenyl)-amine [69];
3-[4-(4-Methyl-2-methylamino-thiazol-5-yl)-pyrimidin-2-ylamino]-phenol [70];
(3-Methoxy-phenyl)-[4-(4-methyl-2-methylamino-thiazol-5-yl)-pyrimidin-2-yl]-amine [73];
[4-(4-Methyl-2-methylamino-thiazol-5-yl)-pyrimidin-2-yl]-(3-trifluoromethyl-phenyl)-amine [75];
(3-Fluoro-phenyl)-[4-(4-methyl-2-methylamino-thiazol-5-yl)-pyrimidin-2-yl]-amine [87];
(4-Methoxy-phenyl)-[4-(4-methyl-2-methylamino-thiazol-5-yl)-pyrimidin-2-yl]-amine [93];
4-{4-[2-(4-Nitro-phenylamino)-thiazol-5-yl]-pyrimidin-2-ylamino}-phenol [95];
4-[4-(2-Amino-4-methyl-thiazol-5-yl)-pyrimidin-2-ylamino]-phenol [98];
N-{3-[4-(2,4-Dimethyl-thiazol-5-yl)-pyrimidin-2-ylamino]-phenyl}-guanidine [99];
{3-[4-(2,4-Dimethyl-thiazol-5-yl)-pyrimidin-2-ylamino]-phenyl}-methanol [100];
[4-(2,4-Dimethyl-thiazol-5-yl)-pyrimidin-2-yl]-(4-pyridin-4-ylmethyl-phenyl)-amine [101];
N,N-Dimethyl-N′-[4-(2,4-dimethyl-thiazol-5-yl)-pyrimidin-2-yl]-benzene-1,4-diamine [103];
{4-[4-(2,4-Dimethyl-thiazol-5-yl)-pyrimidin-2-ylamino]-phenyl}-trimethyl-ammonium [104];
[4-(2-Amino-4-methyl-thiazol-5-yl)-pyrimidin-2-yl]-(3-nitro-phenyl)-amine [105];
N-[4-(2-Amino-4-methyl-thiazol-5-yl)-pyrimidin-2-yl]-N′,N′-dimethyl-benzene-1,4-diamine [106];
[4-(2-Amino-4-methyl-thiazol-5-yl)-pyrimidin-2-yl]-(3-methoxy-phenyl)-amine [108];
[4-(2-Amino-4-methyl-thiazol-5-yl)-pyrimidin-2-yl]-(3-fluoro-phenyl)-amine [109];
[4-(2-Amino-4-methyl-thiazol-5-yl)-pyrimidin-2-yl]-(4-trifluoromethyl-phenyl)-amine [110];
[4-(2-Amino-4-methyl-thiazol-5-yl)-pyrimidin-2-yl]-(4-methoxy-phenyl)-amine [111];
[4-(2-Amino-4-methyl-thiazol-5-yl)-pyrimidin-2-yl]-(3-chloro-phenyl)-amine [112];
[4-(2-Amino-4-methyl-thiazol-5-yl)-pyrimidin-2-yl]-(3-iodo-phenyl)-amine [113];
3-[4-(2-Amino-4-methyl-thiazol-5-yl)-pyrimidin-2-ylamino]-phenol [116];
[4-(2-Amino-4-methyl-thiazol-5-yl)-pyrimidin-2-yl]-(4-iodo-3-nitro-phenyl)-amine [117];
2-{4-[4-(2-Amino-4-methyl-thiazol-5-yl)-pyrimidin-2-ylamino]-phenyl}-ethanol [118];
[4-(2-Amino-4-methyl-thiazol-5-yl)-pyrimidin-2-yl]-(3-bromo-phenyl)-amine [119];
N¹-[4-(2,4-Dimethyl-thiazol-5-yl)-pyrimidin-2-yl]-4-[ B-(phenoxy)-triethylamine]-amine [122];
2-{4-[4-(2,4-Dimethyl-thiazol-5-yl)-pyrimidin-2-ylamino]-phenyl}-ethanol [123];
(3,4-Dimethoxy-phenyl)-[4-(2,4-dimethyl-thiazol-5-yl)-pyrimidin-2-yl]-amine [125];
5-[4-(2,4-Dimethyl-thiazol-5-yl)-pyrimidin-2-ylamino]-2-methoxy-phenol [126];
N⁴-[4-(2,4-Dimethyl-thiazol-5-yl)-pyrimidin-2-yl]-N¹,N¹-dimethyl-2-nitro-benzene-1,4-diamine [127];
2-[N-(4-N,N-Dimethylamino-3-chlorophenyl)]-4-(2,4-dimethylthiazol-5-yl)-pyrimidineamine [128];
N¹-[4-(2,4-Dimethyl-thiazol-5-yl)-pyrimidin-2-yl]-4-methoxy-N³,N³-dimethyl-benzene-1,3-diamine [130];
N,N-Dimethyl-N′-[4-(4-methyl-2-methylamino-thiazol-5-yl)-pyrimidin-2-yl]-benzene- 1,4-diamine [131];
(4-Iodo-3-nitro-phenyl)-[4-(4-methyl-2-methylamino-thiazol-5-yl)-pyrimidin-2-yl]-amine [132];
[4-(2-Ethylamino-4-methyl-thiazol-5-yl)-pyrimidin-2-yl]-(3-nitro-phenyl)-amine [133]
[4-(2-Dimethylamino-4-methyl-thiazol-5-yl)-pyrimidin-2-yl]-(3-nitro-phenyl)-amine [140];
(3-Chloro-phenyl)-[4-(2-dimethylamino-4-methyl-thiazol-5-yl)-pyrimidin-2-yl]-amine [143];
2-{4-Methyl-5-[2-(3-nitro-phenylamino)-pyrimidin-4-yl]-thiazol-2-ylamino}-ethanol [144];
2-{5-[2-(4-Fluoro-phenylamino)-pyrimidin-4-yl]-4-methyl-thiazol-2-ylamino}-ethanol [145];
4-[4-(2,4-Dimethyl-thiazol-5-yl)-pyrimidin-2-ylamino]-benzenesulfonic acid [148];
N-[4-(2-Amino-4-methyl-thiazol-5-yl)-pyrimidin-2-yl]-benzene-1,3-diamine [149].
[4-(2,4-Dimethyl-thiazol-5-yl)-pyrimidin-2-yl]-phenyl-amine [150]; and
[4-(2-Amino-4-methyl-thiazol-5-yl)-pyrimidin-2-yl]-(4-nitro-phenyl)-amine [151];

19. The method of claim 1, wherein said compound of formula I selected from the group consisting of:
[4-(2,4-Dimethyl-thiazol-5-yl)-pyrimidin-2-yl]-(4-iodo-phenyl)-amine [21];
4-[4-(2,4-Dimethyl-thiazol-5-yl)-pyrimidin-2-ylamino]-phenol [28];
(4-Fluoro-phenyl)-[4-(4-methyl-2-methylamino-thiazol-5-yl)-pyrimidin-2-yl]-amine [47];
3-[4-(4-Methyl-2-methylamino-thiazol-5-yl)-pyrimidin-2-ylamino]-phenol [70];
N,N-Dimethyl-N′-[4-(2,4-dimethyl-thiazol-5-yl)-pyrimidin-2-yl]-benzene-1,4-diamine [103];
[4-(2-Amino-4-methyl-thiazol-5-yl)-pyrimidin-2-yl]-(3-nitro-phenyl)-amine [105];
(3,4-Dimethoxy-phenyl)-[4-(2,4-dimethyl-thiazol-5-yl)-pyrimidin-2-yl]-amine [125];
2-[N-(4-N,N-Dimethylamino-3-chlorophenyl)]-4-(2,4-dimethylthiazol-5-yl)-pyrimidineamine [128]

20. The method of claim 19, wherein said compound of formula I is selected from the group consisting of:
[4-(2,4-Dimethyl-thiazol-5-yl)-pyrimidin-2-yl]-(4-iodo-phenyl)-amine [21];
4-[4-(2,4-Dimethyl-thiazol-5-yl)-pyrimidin-2-ylamino]-phenol [28];
(4-Fluoro-phenyl)-[4-(4-methyl-2-methylamino-thiazol-5-yl)-pyrimidin-2-yl]-amine [47];
N,N-Dimethyl-N′-[4-(2,4-dimethyl-thiazol-5-yl)-pyrimidin-2-yl]-benzene-1,4-diamine [103];
[4-(2-Amino-4-methyl-thiazol-5-yl)-pyrimidin-2-yl]-(3-nitro-phenyl)-amine [105];
(3,4-Dimethoxy-phenyl)-[4-(2,4-dimethyl-thiazol-5-yl)-pyrimidin-2-yl]-amine [125];
2-[N-(4-N,N-Dimethylamino-3-chlorophenyl)]-4-(2,4-dimethylthiazol-5-yl)-pyrimidineamine [128];

21. The method of claim 1, wherein the viral disorder is selected from human cytomegalovirus (HCMV), herpes simplex virus type 1 (HSV-1), human immunodeficiency virus type 1 (HIV-1), and varicella zoster virus (VZV).

22. The method of claim 1, wherein said one or more compounds are administered in an amount sufficient to inhibit at least one CDK enzyme.

23. The method of claim 22 wherein the CDK enzyme is CDK2, CDK7, CDK8 and/or CDK9.

24. The method of claim 1, wherein said compound of formula I is administered in combination with a pharmaceutically acceptable excipient, diluent or carrier.

25. The method of claim 1, wherein said compound is administered in combination with one or more other antiviral compounds.
